(19) United States
(12) Patent Application Publication
(10) Pub. No.: US 2025/0115917 A1
(43) Pub. Date: Apr. 10, 2025

(54) MODULATORY POLYNUCLEOTIDES

(71) Applicant: VOYAGER THERAPEUTICS, INC., Lexington, MA (US)

(72) Inventors: Jinzhao Hou, Lexington, MA (US); Mathieu E. Nonnenmacher, Boston, MA (US); Pengcheng Zhou, Lexington, MA (US)

(21) Appl. No.: 18/775,835

(22) Filed: Jul. 17, 2024

Related U.S. Application Data
(63) Continuation of application No. 17/497,834, filed on Oct. 8, 2021, now Pat. No. 12,071,625.
Application No. 17/497,834 is a continuation of application No. 16/738,262, filed on Jan. 9, 2020, now Pat. No. 11,198,873.
Application No. 16/738,262 is a continuation of application No. 15/526,697, filed on May 12, 2017, now Pat. No. 10,570,395.
(60) Provisional application No. 62/234,477, filed on Sep. 29, 2015.
(60) Provisional application No. 62/212,004, filed on Aug. 31, 2015.
(60) Provisional application No. 62/079,590, filed on Nov. 14, 2014.

(51) Int. Cl. C12N 15/113; C12N 7/00; C12N 15/11; C12N 15/86

(52) U.S. Cl. CPC C12N 15/1137; C12N 7/00; C12N 15/111; C12N 15/86; C12Y 115/01001; C12N 2310/14; C12N 2310/141; C12N 2310/34; C12N 2310/531; C12N 2330/50; C12N 2750/14121; C12N 2750/14143

(57) ABSTRACT

The invention relates to compositions and methods for the preparation, manufacture and therapeutic use of modulatory polynucleotides.

### CROSS REFERENCE TO RELATED APPLICATIONS

This application is a continuation of U.S. patent application Ser. No. 17/497,834 filed Oct. 8, 2021, which is a continuation application which claims the benefit of U.S. patent application Ser. No. 16/738,262 filed Jan. 9, 2020, entitled Modulatory Polynucleotides; which is a continuation application which claims the benefit of U.S. patent application Ser. No. 15/526,697 filed May 12, 2017, entitled Modulatory Polynucleotides; which is a national stage filing under 35 U.S.C. § 371 of International Application No. PCT/US2015/060564 filed Nov. 13, 2015, which claims priority to U.S. Provisional Patent Application No. 62/079,590, entitled Modulatory Polynucleotides, filed Nov. 14, 2014, U.S. Provisional Patent Application No. 62/212,004, entitled Modulatory Polynucleotides, filed Aug. 31, 2015, U.S. Provisional Patent Application No. 62/234,477, entitled Modulatory Polynucleotides, filed Sep. 29, 2015; the contents of each of which are herein incorporated by reference in their entirety.

### REFERENCE TO THE SEQUENCE LISTING

The present application is being filed along with a Sequence Listing in electronic XML file format. The Sequence Listing is provided as a file entitled V2071-1014USCON3_SL.xml, created on Jul. 16, 2024, which is 1,321,832 bytes in size. The information in the electronic format of the sequence listing is incorporated herein by reference in its entirety.

### FIELD OF THE DISCLOSURE

The present disclosure relates to compositions, methods, processes, kits and devices for the design, preparation, manufacture and/or formulation of modulatory polynucleotides. In some embodiments such modulatory polynucleotides may be encoded by or within recombinant adeno-associated viruses (AAV) and may comprise artificial microRNAs, artificial pre-microRNAs and/or artificial pri-microRNAs.

### BACKGROUND

MicroRNAs (or miRNAs or miRs) are small, non-coding, single stranded ribonucleic acid molecules (RNAs), which are usually 19-25 nucleotides in length. More than a thousand microRNAs have been identified in mammalian genomes. The mature microRNAs primarily bind to the 3′ untranslated region (3′-UTR) of target messenger RNAs (mRNAs) through partially or fully pairing with the complementary sequences of target mRNAs, promoting the degradation of target mRNAs at a post-transcriptional level, and in some cases, inhibiting the initiation of translation. MicroRNAs play a critical role in many key biological processes, such as the regulation of cell cycle and growth, apoptosis, cell proliferation and tissue development.

miRNA genes are generally transcribed as long primary transcripts of miRNAs (i.e. pri-miRNAs). The pri-miRNA is cleaved into a precursor of a miRNA (i.e. pre-miRNA) which is further processed to generate the mature and functional miRNA.

While many target expression strategies employ nucleic acid based modalities, there remains a need for improved nucleic acid modalities which have higher specificity and with fewer off target effects.

The present invention provides such improved modalities in the form of artificial pri-, pre- and mature microRNA constructs and methods of their design. These novel constructs may be synthetic stand-alone molecules or be encoded in a plasmid or expression vector for delivery to cells. Such vectors include, but are not limited to adeno-associated viral vectors such as vector genomes of any of the AAV serotypes or other viral delivery vehicles such as lentivirus, etc.

### SUMMARY

Described herein are compositions, methods, processes, kits and devices for the design, preparation, manufacture and/or formulation of modulatory polynucleotides.

In some embodiments such modulatory polynucleotides may be encoded by or contained within plasmids or vectors or recombinant adeno-associated viruses (AAV) and may comprise artificial microRNAs, artificial pre-microRNAs and/or artificial pri-microRNAs.

The details of various embodiments of the invention are set forth in the description below. Other features, objects, and advantages of the invention will be apparent from the description and the drawings, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a schematic of an artificial pri-microRNA encoded in an AAV vector according to the present invention.

FIG. 2 is a histogram showing the activity of the pri-mRNA constructs encoded in AAV vectors.

FIG. 3 is a histogram showing the activity in HEK293T cells of the guide strand of the modulatory polynucleotides encoded in AAV vectors.

FIG. 4 is a histogram showing the activity in HEK293T cells of the passenger strand of the modulatory polynucleotides encoded in AAV vectors.

FIG. 5 is a histogram showing the activity in HeLa cells of the guide strand of the modulatory polynucleotides encoded in AAV vectors.

FIG. 6 is a histogram showing the activity in HeLa cells of the passenger strand of the modulatory polynucleotides encoded in AAV vectors.

FIG. 7 is a histogram for the quantification of expressed intracellular AAV DNA.

FIG. 8 is a histogram showing the activity in human motor neurons of the constructs encoded in AAV vectors.

FIG. 9 is a chart showing the dose-dependent silencing of SOD1 in U251MG cells.

FIG. 10 is a chart showing the dose-dependent silencing of SOD1 in human astrocyte cells.

FIG. 11 is a chart showing the time course of the silencing of SOD1 in U251MG cells.

FIG. 12A, FIG. 12B and FIG. 12C are charts showing the dose-dependent effects of a construct. FIG. 12A shows the relative SOD1 expression. FIG. 12B shows the percent of guide strand. FIG. 12C shows the percent of the passenger strand.

FIG. 13 is a diagram showing the location of the modulatory polynucleotide (MP) in relation to the ITRs, the intron (I) and the polyA (P).

### DETAILED DESCRIPTION

### Compositions of the Invention

According to the present invention, modulatory polynucleotides are provided which function as artificial microRNAs. As used herein a “modulatory polynucleotide” is any nucleic acid polymer which functions to modulate (either increase or decrease) the level or amount of a target gene. Modulatory polynucleotides include precursor molecules which are processed inside the cell prior to modulation. Modulatory polynucleotides or the processed forms thereof may be encoded in a plasmid, vector, genome or other nucleic acid expression vector for delivery to a cell.

In some embodiments modulatory polynucleotides are designed as primary microRNA (pri-miRs) or precursor microRNAs (pre-miRs) which are processed within the cell to produce highly specific artificial microRNAs.

The modulatory polynucleotides, especially the artificial microRNAs of the invention, may be designed based on the sequence or structure scaffold of a canonical or known microRNA, pri-microRNA or pre-microRNA. Such sequences may correspond to any known microRNA or its precursor such as those taught in US Publication US2005/0261218 and US Publication US2005/0059005, the contents of which are incorporated herein by reference in their entirety.

microRNAs (or miRNA or miRs) are 19-25 nucleotide long noncoding RNAs that bind to the 3′UTR of nucleic acid molecules and down-regulate gene expression either by reducing nucleic acid molecule stability or by inhibiting translation. The modulatory polynucleotides of the invention may comprise one or more microRNA sequences, microRNA seeds or artificial microRNAs, e.g., sequences which function as a microRNA.

A microRNA sequence comprises a “seed” region, i.e., a sequence in the region of positions 2-9 of the mature microRNA, which sequence has perfect Watson-Crick complementarity to the miRNA target sequence. A microRNA seed may comprise positions 2-8 or 2-7 or 2-9 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. In naturally occurring microRNA, the bases of the microRNA seed have complete complementarity with the target sequence.

As taught herein, design parameters, or rules, have been identified and applied to design modulatory polynucleotides (e.g., artificial microRNAs) which have superior target gene modulatory properties with limited off target effects.

In one embodiment, the molecular scaffold of the modulatory polynucleotide described herein may be designed and optimized to create a modulatory polynucleotide that has the desired target gene modulatory properties. As a non-limiting example, the modulatory polynucleotide can have superior target gene modulatory properties with limited off target effects.

In one embodiment, the modulatory polynucleotides of the invention, such as artificial miRs, are comprised of modular elements or sequence motifs assembled according to a set of rules that result in highly specific target recognition and low guide/passenger ratio. Such modules or sequence motifs include, but are not limited to, double stranded regions, flanking regions, loops, optimized loops, UGUG loops, GU domains, spacers (to control proximal and distal motif or module spacing or to introduce structural elements such as turns, loops or bulges), CNNC motifs, and thermodynamic asymmetry regions which may embrace loops, bulges, mismatches, wobbles, and/or combinations thereof. Non limiting examples of rules which may be applied alone or in combination when constructing artificial miRs include those taught in Seitz et al. Silence 2011, 2:4; Gu, et al., Cell 151, 900-911, Nov. 9, 2012; Schwartz, et al., Cell, Vol. 115, 199-208, Oct. 17, 2003; Park, et al., Nature, Vol. 475, 101, 14 Jul. 2011; Ketley et al., 2013, PLoS ONE 8(6); Liu, et al., Nucleic Acids Research, 2008, Vol. 36, No. 9 2811-2824; Dow, et al., 2013, Nat Protoc.; 7(2): 374-393. doi:10.1038/nprot.2011.446; Auyeung, et al., Cell 152, 844-858, Feb. 14, 2013; Gu et al., Cell 2012 Nov. 9, 151(4):900-11; Fellmann et al. Molecular Cell 41, 733-746, 2011; Han et al. Cell 125, 887-907, 2006; Betancur et al. Frontiers in Genetics, Vol. 3, Art. 127, 1-6 Jul. 2012; Schwarz et al. Cell Vol 115, 199-208, 2003; the contents of each of which are herein incorporated by reference in their entirety.

In addition to the modules or sequence motifs, modulatory polynucleotides comprise at least one of or both a passenger and guide strand. The passenger and guide strand may be positioned or located on the 5′ arm or 3′ arm of a stem loop structure of the modulatory polynucleotide.

In one embodiment, the 3′ stem arm of the modulatory polynucleotides may have 11 nucleotides downstream of the 3′ end of the guide strand which have complementarity to the 11 of the 13 nucleotides upstream of the 5′ end of the passenger strand in the 5′ stem arm.

In one embodiment, the modulatory polynucleotides may have a cysteine which is 6 nucleotides downstream of the 3′ end of the 3′ stem arm of the modulatory polynucleotide.

In one embodiment, the modulatory polynucleotides comprise a miRNA seed match for the guide strand. In another embodiment, the modulatory polynucleotides comprise a miRNA seed match for the passenger strand. In yet another embodiment, the modulatory polynucleotides do no comprise a seed match for the guide or passenger strand.

In one embodiment, the modulatory polynucleotides may have almost no significant full-length off targets for the guide strand. In another embodiment, the modulatory polynucleotides may have almost no significant full-length off targets for the passenger strand. In yet another embodiment, the modulatory polynucleotides may have almost no significant full-length off targets for the guide strand or the passenger strand.

In one embodiment, the modulatory polynucleotides may have high activity in vitro. In another embodiment, the modulatory polynucleotides may have low activity in vitro. In yet another embodiment, the modulatory polynucleotides may have high guide strand activity and low passenger strand activity in vitro.

In one embodiment, the modulatory polynucleotides have a high guide strand activity and low passenger strand activity in vitro. The target knock-down (KD) by the guide strand may be at least 60%, 65%, 70%, 75%, 80%, 85%, 90%, 95%, 99%, 99.5% or 100%. The target knock-down by the guide strand may be 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%.

In one embodiment, the IC50 of the passenger strand for the nearest off target is greater than 100 multiplied by the IC50 of the guide strand for the target. As a non-limiting example, if the IC50 of the passenger strand for the nearest off target is greater than 100 multiplied by the IC50 of the guide strand for the target then the modulatory polynucleotide is said to have high guide strand activity and a low passenger strand activity in vitro.

In one embodiment, the 5′ processing of the guide strand has a correct start (n) at the 5′ end at least 75%, 80%, 85%, 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo.

In one embodiment, the guide-to-passenger (G:P) strand ratio is 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. As a non-limiting example, the guide-to-passenger strand ratio is 80:20 in vitro. As a non-limiting example, the guide-to-passenger strand ratio is 80:20 in vivo.

In one embodiment, the integrity of the vector genome is at least 60%, 65%, 70%, 75%, 80%, 85%, 90%, 95%, 99% or more than 99% of the full length of the construct.

### Modulatory Polynucleotides

In one embodiment, any of the known RNAi constructs or RNAi agents may serve as the starting construct for the design of the passenger and/or guide strand of a modulatory polynucleotides or artificial microRNAs of the invention. These include canonical siRNAs, small interfering RNAs (siRNA), double stranded RNAs (dsRNAs), inverted repeats, short hairpin RNAs (shRNAs), small temporally regulated RNAs (stRNA), clustered inhibitory RNAs (cRNAs), including radial clustered inhibitory RNA, asymmetric clustered inhibitory RNA, linear clustered inhibitory RNA, and complex or compound clustered inhibitory RNA, dicer substrates, DNA-directed RNAi (ddRNAi), single-stranded RNAi (ssRNAi), microRNA (miRNA) antagonists, microRNA mimics, microRNA agonists, blockmirs (a.k.a. Xmirs), microRNA mimetics, microRNA addbacks, supermiRs, the oligomeric constructs disclosed in PCT Publication WO/2005/013901 the contents of which are incorporated herein in their entirety, tripartite RNAi constructs such as those disclosed in US Publication 20090131360, the contents of which are incorporated herein in their entirety, the solo-rxRNA constructs disclosed in PCT Publication WO/2010/011346, the contents of which are incorporated herein by reference in their entirety; the sd-rxRNA constructs disclosed in PCT Publication WO/2010/033247 the contents of which are incorporated herein by reference in their entirety, dual acting RNAi constructs which reduce RNA levels and also modulate the immune response as disclosed in PCT Publications WO/2010/002851 and WO/2009/141146 the contents of which are incorporated herein by reference in their entirety and antigene RNAs (agRNA) or small activating RNAs (saRNAs) which increase expression of the target to which they are designed disclosed in PCT Publications WO/2006/130201, WO/2007/086990, WO/2009/046397, WO/2009/149182, WO/2009/086428 the contents of which are incorporated herein by reference in their entirety.

Likewise, any pri- or pre-microRNA precursor of the above listed microRNA may also serve as the molecular scaffold of the modulatory polynucleotides of the invention.

In one embodiment, the starting construct may be derived from any relevant species such as, not limited to, mouse, rat, dog, monkey or human.

In one embodiment, the modulatory polynucleotide may be located in an expression vector downstream of a promoter such as, but not limited to, CMV, U6, CBA or a CBA promoter with a SV40 intron. Further, the modulatory polynucleotide may also be located upstream of the polyadenylation sequence in an expression vector. As a non-limiting example, the modulatory polynucleotide may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the modulatory polynucleotide may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the modulatory polynucleotide may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the modulatory polynucleotide may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In one embodiment, the modulatory polynucleotide may be located upstream of the polyadenylation sequence in an expression vector. Further, the modulatory polynucleotide may be located downstream of a promoter such as, but not limited to, CMV, U6, CBA or a CBA promoter with a SV40 intron in an expression vector. As a non-limiting example, the modulatory polynucleotide may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the modulatory polynucleotide may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the modulatory polynucleotide may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the modulatory polynucleotide may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In one embodiment, the modulatory polynucleotide may be located in a scAAV.

In one embodiment, the modulatory polynucleotide may be located in an ssAAV.

In one embodiment, the modulatory polynucleotide may be located near the 5′ end of the flip ITR in an expression vector. In another embodiment, the modulatory polynucleotide may be located near the 3′ end of the flip ITR in an expression vector. In yet another embodiment, the modulatory polynucleotide may be located near the 5′ end of the flop ITR in an expression vector. In yet another embodiment, the modulatory polynucleotide may be located near the 3′ end of the flop ITR in an expression vector. In one embodiment, the modulatory polynucleotide may be located between the 5′ end of the flip ITR and the 3′ end of the flop ITR in an expression vector. In one embodiment, the modulatory polynucleotide may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the modulatory polynucleotide may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the modulatory polynucleotide may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR) in an expression vector. As another non-limiting example, the modulatory polynucleotide may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR) in an expression vector. As another non-limiting example, the modulatory polynucleotide may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the modulatory polynucleotide may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the modulatory polynucleotide may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR) in an expression vector.

### Molecular Scaffolds

In some embodiments the starting molecular scaffold of the modulatory polynucleotide is a known or wild type pri- or pre-microRNA. In other embodiments the molecular scaffold of the modulatory polynucleotides are designed ab initio. (See Cullen, Gene Therapy (2006) 13, 503-508 work with miR30; Chung, et al., Nucleic Acids Research, 2006, Vol. 34, No. 7 working with miR-155; the contents of which are herein incorporated by reference in their entirety).

As used herein a “molecular scaffold” is a framework or starting molecule that forms the sequence or structural basis against which to design or make a subsequent molecule.

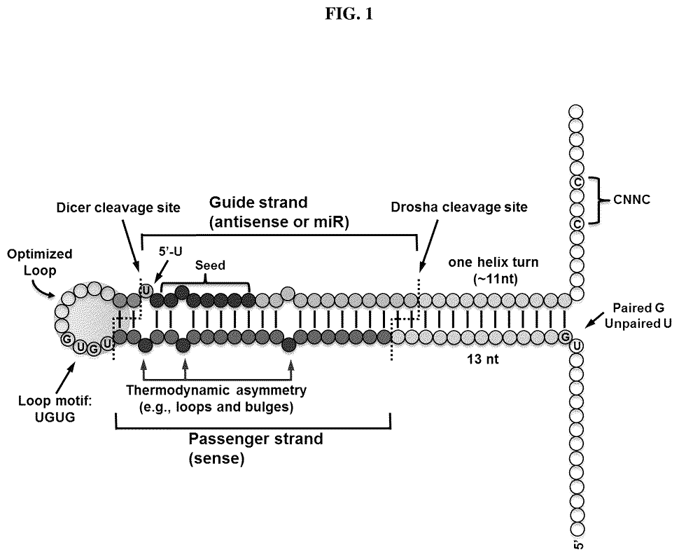

Turning to FIG. 1. The modulatory polynucleotides of the present invention may be designed as a pri-miR as shown. In the figure, a pri-miR molecular scaffold is shown. The modulatory polynucleotide which comprises the payload (e.g., siRNA, miRNA or other RNAi agent described herein) comprises a leading 5′ flanking sequence which may be of any length and may be derived in whole or in part from wild type microRNA sequence or be completely artificial.

Likewise, a 3′ flanking sequence shown in the figure may mirror the 5′ flanking sequence in size and origin. Either flanking sequence may be absent. The 3′ flanking sequence may optionally contain one or more CNNC motifs, where “N” represents any nucleotide.

Forming the stem of the stem loop structure shown is a minimum of at least one payload sequence. In some embodiments the payload sequence comprises at least one nucleic acid sequence which is in part complementary or will hybridize to the target sequence. In some embodiments the payload is a wild type microRNA. In some embodiments the payload is an siRNA molecule or fragment of an siRNA molecule. In some embodiments the payload is a substantially double stranded construct which may comprise one or more microRNAs, artificial microRNAs or siRNAs.

In some embodiments the 5′ arm of the stem loop comprises a passenger strand. This strand is also known as the sense strand in that it reflects an identity to a target. The passenger strand may be between 15-30 nucleotides in length. It may be 15, 16, 17, 18, 19, 20, 21, 22, 23, 24, 25, 26, 27, 28, 29 or 30 nucleotides in length.

In some embodiments the 3′ arm of the stem loop comprises a guide strand. This strand is also known as the antisense strand in that it reflects homology to a target. The guide strand may be between 15-30 nucleotides in length, 21-25 nucleotides or 22 nucleotides in length. It may be 15, 16, 17, 18, 19, 20, 21, 22, 23, 24, 25, 26, 27, 28, 29 or 30 nucleotides in length. The guide strand, in some instances, comprises a “G” nucleotide at the 5′ most end.

In some embodiments, where the guide strand comprises a microRNA, or artificial microRNAs, the guide strand may comprise one or more microRNA seed sequences. The seed sequence may be located at positions 2-7, 2-8 or 2-9 of the guide strand relative to the first 5′ nucleotide of the guide strand or relative to a dicer cleavage site.

In other embodiments, the passenger strand may reside on the 3′ arm while the guide strand resides on the 5′ arm of the stem of the stem loop structure.

The passenger and guide strands may be completely complementary across a substantial portion of their length. In other embodiments the passenger strand and guide strand may be at least 70, 80, 90, 95 or 99% complementary across independently at least 50, 60, 70, 80, 85, 90, 95, or 99% of the length of the strands.

Neither the identity of the passenger strand nor the homology of the guide strand need be 100% complementary to the target.

Separating the passenger and guide strand of the stem loop structure is a loop (also known as a loop motif). The loop may be of any length, between 4-30 nucleotides, between 4-20 nucleotides, between 4-15 nucleotides, between 5-15 nucleotides, between 6-12 nucleotides, 6 nucleotides, 7, nucleotides, 8 nucleotides, 9 nucleotides, 10 nucleotides, 11 nucleotides, and/or 12 nucleotides.

In some embodiments the loop comprises at least one UGUG motif. In some embodiments, the UGUG motif is located at the 5′ terminus of the loop.

Spacer regions may be present in the modulatory polynucleotide to separate one or more modules from one another. There may be one or more such spacer regions present.

In one embodiment a spacer region of between 8-20, i.e., 8, 9, 10, 11, 12, 13, 14, 15, 16, 17, 18, 19, or 20 nucleotides may be present between the passenger strand and a flanking sequence.

In one embodiment, the spacer is 13 nucleotides and is located between the 5′ terminus of the passenger strand and a flanking sequence. In one embodiment a spacer is of sufficient length to form approximately one helical turn of the sequence.

In one embodiment a spacer region of between 8-20, i.e., 8, 9, 10, 11, 12, 13, 14, 15, 16, 17, 18, 19, or 20 nucleotides may be present between the guide strand and a flanking sequence.

In one embodiment, the spacer sequence is between 10-13, i.e., 10, 11, 12 or 13 nucleotides and is located between the 3′ terminus of the guide strand and a flanking sequence. In one embodiment a spacer is of sufficient length to form approximately one helical turn of the sequence.

In one embodiment the modulatory polynucleotide comprises at least one UG motif at the base of the stem whereby the G nucleotide is paired and the U nucleotide is unpaired. In some embodiments the unpaired U nucleotide is located in a flanking sequence.

In one embodiment, the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a passenger strand and the 3′ arm comprises the guide strand. In another non-limiting example, the 5′ arm comprises the guide strand and the 3′ arm comprises the passenger strand.

In one embodiment, the 5′ arm, payload (e.g., passenger and/or guide strand), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In one embodiment, the passenger strand sequence may be altered (e.g., substituting 1 or more nucleotides, adding nucleotides and/or deleting nucleotides). As a non-limiting example, the passenger strand sequence may comprise 1 or 2 substitutions within the last 4 nucleotides of the sequence (e.g., C substituted for a G). As another non-limiting example, the passenger strand sequence may comprise 1 or 2 substitutions within the 7-15 nucleotides from the 5′ end of the sequence (e.g., U substituted for an A or C substituted for a G).

In one embodiment, the 3′ arm strand sequence may be altered (e.g., substituting 1 or more nucleotides, adding nucleotides and/or deleting nucleotides). As a non-limiting example, the sequence of the 3′ arm may comprise 1 or 2 substitutions within the first 4 nucleotides of the sequence (e.g., A substituted for a U).

In one embodiment, the molecular scaffold of the payload construct may comprise a 5′ flanking region, a loop motif and a 3′ flanking region. Between the 5′ flanking region and the loop motif may be a first payload region and between the loop motif and the 3′ flanking region may be a second payload region. The first and second payload regions may comprise siRNA, miRNA or other RNAi agents, fragments or variants described herein. The first and second payload regions may also comprise a sequence which is the same, different or complementary to each other. As a non-limiting example, the first payload region sequence may be a passenger strand of a siRNA construct and the second payload region sequence may be a guide strand of an siRNA construct. The passenger and guide sequences may be substantially complementary to each other. As another non-limiting example, the first payload region sequence may be a guide strand of a siRNA construct and the second payload region sequence may be a passenger strand of an siRNA construct. The passenger and guide sequences may be substantially complementary to each other.

In one embodiment, the molecular scaffold of the modulatory polynucleotides described herein comprise a 5′ flanking region, a loop region and a 3′ flanking region. Non-limiting examples of the sequences for the 5′ flanking region, loop region and the 3′ flanking region which may be used in the molecular scaffolds described herein are shown in Tables 1-3.

TABLE 1
5′ Flanking Regions for Molecular Scaffold

| | | 5′ |
|---|---|---|
| | | Flanking |
| 5′ Flanking | | Region |
| Region Name | 5′ Flanking Region Sequence | SEQ ID |
| 5F1 | UUUAUGCCUCAUCCUCUGAGUGCUGAA | 1 |
| | GGCUUGCUGUAGGCUGUAUGCUG | |
| 5F2 | GUGCUGGGCGGGGGGCGGCGGGCCCUC | 2 |
| | CCGCAGAACACCAUGCGCUCUUCGGAA | |
| 5F3 | GAAGCAAAGAAGGGGCAGAGGGAGCCC | 3 |
| | GUGAGCUGAGUGGGCCAGGGACUGGGA | |
| | GAAGGAGUGAGGAGGCAGGGCCGGCAU | |
| | GCCUCUGCUGCUGGCCAGA | |
| 5F4 | GUGCUGGGCGGGGGGCGGCGGGCCCUC | 4 |
| | CCGCAGAACACCAUGCGCUCUUCGGGA | |

TABLE 2
Loop Motif Regions for Molecular Scaffold

| | | Loop Motif |
|---|---|---|
| Loop Motif | | Region |
| Region Name | Loop Motif Region Sequence | SEQ ID |
| L1 | UGUGACCUGG | 5 |
| L2 | UGUGAUUUGG | 6 |
| L3 | UAUAAUUUGG | 7 |
| L4 | CCUGACCCAGU | 8 |
| L5 | GUCUGCACCUGUCACUAG | 9 |

TABLE 3
3′Flanking Regions for Molecular Scaffold

| | | 3′ |
|---|---|---|
| | | Flanking |
| 3′ Flanking | | Region |
| Region Name | 3′ Flanking Region Sequence | SEQ ID |
| 3F1 | AGUGUAUGAUGCCUGUUACUAGCAUUC | 10 |
| | ACAUGGAACAAAUUGCUGCCGUG | |
| 3F2 | CUGAGGAGCGCCUUGACAGCAGCCAUG | 11 |
| | GGAGGGCCGCCCCCUACCUCAGUGA | |
| 3F3 | CUGUGGAGCGCCUUGACAGCAGCCAUG | 12 |
| | GGAGGGCCGCCCCCUACCUCAGUGA | |
| 3F4 | UGGCCGUGUAGUGCUACCCAGCGCUGG | 13 |
| | CUGCCUCCUCAGCAUUGCAAUUCCUCU | |
| | CCCAUCUGGGCACCAGUCAGCUACCCU | |
| | GGUGGGAAUCUGGGUAGCC | |
| 3F5 | GGCCGUGUAGUGCUACCCAGCGCUGGC | 14 |
| | UGCCUCCUCAGCAUUGCAAUUCCUCUC | |
| | CCAUCUGGGCACCAGUCAGCUACCCUG | |
| | GUGGGAAUCUGGGUAGCC | |
| 3F6 | UCCUGAGGAGCGCCUUGACAGCAGCCA | 810 |
| | UGGGAGGGCCGCCCCCUACCUCAGUGA | |

Any of the regions described in Tables 1-3 may be used in the molecular scaffolds described herein.

In one embodiment, the molecular scaffold may comprise one 5′ flanking region listed in Table 1. As a non-limiting example, the molecular scaffold may comprise the 5′ flanking region 5F1, 5F2, 5F3 or 5F4.

In one embodiment, the molecular scaffold may comprise one loop motif region listed in Table 2. As a non-limiting example, the molecular scaffold may comprise the loop motif region L1, L2, L3, L4 or L5.

In one embodiment, the molecular scaffold may comprise one 3′ flanking region listed in Table 3. As a non-limiting example, the molecular scaffold may comprise the 3′ flanking region 3F1, 3F2, 3F3, 3F4, 3F5 or 3F6.

In one embodiment, the molecular scaffold may comprise at least one 5′ flanking region and at least one loop motif region as described in Tables 1 and 2. As a non-limiting example, the molecular scaffold may comprise 5F1 and L1, 5F1 and L2, 5F1 and L3, 5F1 and L4, 5F1 and L5, 5F2 and L1, 5F2 and L2, 5F2 and L3, 5F2 and L4, 5F2 and L5, 5F3 and L1, 5F3 and L2, 5F3 and L3, 5F3 and L4, 5F3 and L5, 5F4 and L1, 5F4 and L2, 5F4 and L3, 5F4 and L4, or 5F4 and L5.

In one embodiment, the molecular scaffold may comprise at least one 3′ flanking region and at least one loop motif region as described in Tables 2 and 3. As a non-limiting example, the molecular scaffold may comprise 3F1 and L1, 3F1 and L2, 3F1 and L3, 3F1 and L4, 3F1 and L5, 3F2 and L1, 3F2 and L2, 3F2 and L3, 3F2 and L4, 3F2 and L5, 3F3 and L1, 3F3 and L2, 3F3 and L3, 3F3 and L4, 3F3 and L5, 3F4 and L1, 3F4 and L2, 3F4 and L3, 3F4 and L4, 3F4 and L5, 3F5 and L1, 3F5 and L2, 3F5 and L3, 3F5 and L4, 3F5 and L5, 3F6 and L1, 3F6 and L2, 3F6 and L3, 3F6 and L4 or 3F6 and L5.

In one embodiment, the molecular scaffold may comprise at least one 5′ flanking region and at least 3′ flanking region as described in Tables 1 and 3. As a non-limiting example, the molecular scaffold may comprise 5F1 and 3F1, 5F1 and 3F2, 5F1 and 3F3, 5F1 and 3F4, 5F1 and 3F5, 5F1 and 3F6, 5F2 and 3F1, 5F2 and 3F2, 5F2 and 3F3, 5F2 and 3F4, 5F2 and 3F5, 5F2 and 3F6, 5F3 and 3F1, 5F3 and 3F2, 5F3 and 3F3, 5F3 and 3F4, 5F3 and 3F5, 5F3 and 3F6, 5F4 and 3F1, 5F4 and 3F2, 5F4 and 3F3, 5F4 and 3F4, 5F4 and 3F5, 5F4 and 3F6.

In one embodiment, the molecular scaffold may comprise at least one 5′ flanking region, at least one loop motif region and at least one 3′ flanking region. As a non-limiting example, the molecular scaffold may comprise 5F1, L1 and 3F1; 5F1, L1 and 3F2; 5F1, L1 and 3F3; 5F1, L1 and 3F4; 5F1, L1 and 3F5; 5F1, L1 and 3F6; 5F2, L1 and 3F1; 5F2, L1 and 3F2; 5F2, L1 and 3F3; 5F2, L1 and 3F4; 5F2, L1 and 3F5; 5F2, L1 and 3F6; 5F3, L1 and 3F1; 5F3, L1 and 3F2; 5F3, L1 and 3F3; 5F3, L1 and 3F4; 5F3, L1 and 3F5; 5F3, L1 and 3F6; 5F4, L1 and 3F1; 5F4, L1 and 3F2; 5F4, L1 and 3F3; 5F4, L1 and 3F4; 5F4, L1 and 3F5; 5F4, L1 and 3F6; 5F1, L2 and 3F1; 5F1, L2 and 3F2; 5F1, L2 and 3F3; 5F1, L2 and 3F4; 5F1, L2 and 3F5; 5F1, L2 and 3F6; 5F2, L2 and 3F1; 5F2, L2 and 3F2; 5F2, L2 and 3F3; 5F2, L2 and 3F4; 5F2, L2 and 3F5; 5F2, L2 and 3F6; 5F3, L2 and 3F1; 5F3, L2 and 3F2; 5F3, L2 and 3F3; 5F3, L2 and 3F4; 5F3, L2 and 3F5; 5F3, L2 and 3F6; 5F4, L2 and 3F1; 5F4, L2 and 3F2; 5F4, L2 and 3F3; 5F4, L2 and 3F4; 5F4, L2 and 3F5; 5F4, L2 and 3F6; 5F1, L3 and 3F1; 5F1, L3 and 3F2; 5F1, L3 and 3F3; 5F1, L3 and 3F4; 5F1, L3 and 3F5; 5F1, L3 and 3F6; 5F2, L3 and 3F1; 5F2, L3 and 3F2; 5F2, L3 and 3F3; 5F2, L3 and 3F4; 5F2, L3 and 3F5; 5F2, L3 and 3F6; 5F3, L3 and 3F1; 5F3, L3 and 3F2; 5F3, L3 and 3F3; 5F3, L3 and 3F4; 5F3, L3 and 3F5; 5F3, L3 and 3F6; 5F4, L3 and 3F1; 5F4, L3 and 3F2; 5F4, L3 and 3F3; 5F4, L3 and 3F4; 5F4, L3 and 3F5; 5F4, L3 and 3F6; 5F1, L4 and 3F1; 5F1, L4 and 3F2; 5F1, L4 and 3F3; 5F1, L4 and 3F4; 5F1, L4 and 3F5; 5F1, L4 and 3F6; 5F2, L4 and 3F1; 5F2, L4 and 3F2; 5F2, L4 and 3F3; 5F2, L4 and 3F4; 5F2, L4 and 3F5; 5F2, L4 and 3F6; 5F3, L4 and 3F1; 5F3, L4 and 3F2; 5F3, L4 and 3F3; 5F3, L4 and 3F4; 5F3, L4 and 3F5; 5F3, L4 and 3F6; 5F4, L4 and 3F1; 5F4, L4 and 3F2; 5F4, L4 and 3F3; 5F4, L4 and 3F4; 5F4, L4 and 3F5; 5F4, L4 and 3F6; 5F1, L5 and 3F1; 5F1, L5 and 3F2; 5F1, L5 and 3F3; 5F1, L5 and 3F4; 5F1, L5 and 3F5; 5F1, L5 and 3F6; 5F2, L5 and 3F1; 5F2, L5 and 3F2; 5F2, L5 and 3F3; 5F2, L5 and 3F4; 5F2, L5 and 3F5; 5F2, L5 and 3F6; 5F3, L5 and 3F1; 5F3, L5 and 3F2; 5F3, L5 and 3F3; 5F3, L5 and 3F4; 5F3, L5 and 3F5; 5F3, L5 and 3F6; 5F4, L5 and 3F1; 5F4, L5 and 3F2; 5F4, L5 and 3F3; 5F4, L5 and 3F4; 5F4, L5 and 3F5; or 5F4, L5 and 3F6.

In one embodiment, the molecular scaffold may comprise one or more linkers known in the art. The linkers may separate regions or one molecular scaffold from another. As a non-limiting example, the molecular scaffold may be polycistronic.

In one embodiment, the modulatory polynucleotide is designed using at least one of the following properties: loop variant, seed mismatch/bulge/wobble variant, stem mismatch, loop variant and vassal stem mismatch variant, seed mismatch and basal stem mismatch variant, stem mismatch and basal stem mismatch variant, seed wobble and basal stem wobble variant, or a stem sequence variant.

In one embodiment, the molecular scaffold may be located downstream of a promoter such as, but not limited to, CMV, U6, CBA or a CBA promoter with a SV40 intron. Further, the molecular scaffold may also be located upstream of the polyadenylation sequence. As a non-limiting example, the molecular scaffold may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence. As another non-limiting example, the molecular scaffold may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence. As a non-limiting example, the molecular scaffold may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence. As another non-limiting example, the molecular scaffold may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence.

In one embodiment, the molecular scaffold may be located upstream of the polyadenylation sequence. Further, the molecular scaffold may be located downstream of a promoter such as, but not limited to, CMV, U6, CBA or a CBA promoter with a SV40 intron. As a non-limiting example, the molecular scaffold may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence. As another non-limiting example, the molecular scaffold may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence. As a non-limiting example, the molecular scaffold may be located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence. As another non-limiting example, the molecular scaffold may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence.

In one embodiment, the molecular scaffold may be located in a scAAV.

In one embodiment, the molecular scaffold may be located in an ssAAV.

In one embodiment, the molecular scaffold may be located near the 5′ end of the flip ITR. In another embodiment, the molecular scaffold may be located near the 3′ end of the flip ITR. In yet another embodiment, the molecular scaffold may be located near the 5′ end of the flop ITR. In yet another embodiment, the molecular scaffold may be located near the 3′ end of the flop ITR. In one embodiment, the molecular scaffold may be located between the 5′ end of the flip ITR and the 3′ end of the flop ITR. In one embodiment, the molecular scaffold may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR. As a non-limiting example, the molecular scaffold may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR). As a non-limiting example, the molecular scaffold may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides upstream from the 5′ or 3′ end of an ITR (e.g., Flip or Flop ITR). As another non-limiting example, the molecular scaffold may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an ITR (e.g., Flip or Flop ITR). As another non-limiting example, the molecular scaffold may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR). As a non-limiting example, the molecular scaffold may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR). As another non-limiting example, the molecular scaffold may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the 5′ or 3′ end of an ITR (e.g., Flip or Flop ITR).

### Expression Vector

In one embodiment, an expression vector (e.g., AAV vector) may comprise at least one of the modulatory polynucleotides comprising at least one of the molecular scaffolds described herein.

In one embodiment, an expression vector may comprise, from ITR to ITR recited 5′ to 3′, an ITR, a promoter, an intron, a modulatory polynucleotide, a polyA sequence and an ITR.

### Genome Size

In one embodiment, the vector genome which comprises a nucleic acid sequence encoding the modulatory polynucleotides described herein may be a single stranded or double stranded vector genome. The size of the vector genome may be small, medium, large or the maximum size. Additionally, the vector genome may comprise a promoter and a polyA tail.

In one embodiment, the vector genome which comprises a nucleic acid sequence encoding the modulatory polynucleotides described herein may be a small single stranded vector genome. A small single stranded vector genome may be 2.7 to 3.5 kb in size such as about 2.7, 2.8, 2.9, 3.0, 3.1, 3.2, 3.3, 3.4, and 3.5 kb in size. As a non-limiting example, the small single stranded vector genome may be 3.2 kb in size. Additionally, the vector genome may comprise a promoter and a polyA tail.

In one embodiment, the vector genome which comprises a nucleic acid sequence encoding the modulatory polynucleotides described herein may be a small double stranded vector genome. A small double stranded vector genome may be 1.3 to 1.7 kb in size such as about 1.3, 1.4, 1.5, 1.6, and 1.7 kb in size. As a non-limiting example, the small double stranded vector genome may be 1.6 kb in size. Additionally, the vector genome may comprise a promoter and a polyA tail.

In one embodiment, the vector genome which comprises a nucleic acid sequence encoding the modulatory polynucleotides described herein may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may comprise a promoter and a polyA tail.

In one embodiment, the vector genome which comprises a nucleic acid sequence encoding the modulatory polynucleotides described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may be 2.0 kb in size. Additionally, the vector genome may comprise a promoter and a polyA tail.

In one embodiment, the vector genome which comprises a nucleic acid sequence encoding the modulatory polynucleotides described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may comprise a promoter and a polyA tail.

In one embodiment, the vector genome which comprises a nucleic acid sequence encoding the modulatory polynucleotides described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may comprise a promoter and a polyA tail.

### Promoters

A person skilled in the art may recognize that a target cell may require a specific promoter including but not limited to a promoter that is species specific, inducible, tissue-specific, or cell cycle-specific Parr et al., Nat. Med. 3:1145-9 (1997); the contents of which are herein incorporated by reference in their entirety).

In one embodiment, the promoter is a promoter deemed to be efficient for the payload in the modulatory polynucleotide.

In one embodiment, the promoter is a promoter deemed to be efficient for the cell being targeted.

In one embodiment, the promoter is a weak promoter which provides expression of a payload for a period of time in targeted tissues such as, but not limited to, nervous system tissues. Expression may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a weak promoter for sustained expression of a payload in nervous tissues.

In one embodiment, the promoter may be a promoter which is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. As a non-limiting example, the promoter is a combination of a 382 nucleotide CMV-enhancer sequence and a 260 nucleotide CBA-promoter sequence.

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in their entirety) such as promoters. Promoters which promote expression in most tissues include, but are not limited to, human elongation factor 1α-subunit (EF1α), immediate-early cytomegalovirus (CMV), chicken β-actin (CBA) and its derivative CAG, the R glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), CaMKII, mGluR2, NFL, NFH, nβ2, PPE, Enk and EAAT2 promoters. A non-limiting example of tissue-specific expression elements for astrocytes include the glial fibrillary acidic protein (GFAP) and EAAT2 promoters. A non-limiting example of a tissue-specific expression element for oligodendrocytes is the myelin basic protein (MBP) promoter.

In one embodiment, the vector genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3). Yu et al. (Molecular Pain 2011, 7:63; the content of which is herein incorporated by reference in its entirety) evaluated the expression of eGFP under the CAG, EFIα, PGK and UBC promoters in rat DRG cells and primary DRG cells using lentiviral vectors and found that UBC showed weaker expression than the other 3 promoters and there was only 10-12% glial expression seen for all promoters. Soderblom et al. (E. Neuro 2015; the contents of which are herein incorporated by reference in its entirety) the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in their entirety) evaluated a HβH construct with a hGUSB promoter, a HSV-1LAT promoter and a NSE promoter and found that the HβH construct showed weaker expression than NSE in mouse brain. Passini and Wolfe (J. Virol. 2001, 12382-12392, the contents of which are herein incorporated by reference in their entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in sensory proprioceptive neurons, brain and spinal cord and NFH is present in the heart. Scn8a is a 470 nucleotide promoter which expresses throughout the DRG, spinal cord and brain with particularly high expression seen in hippocampal neurons and cerebellar Purkinje cells, cortex, thalamus and hypothalamus (See e.g., Drews et al. 2007 and Raymond et al. 2004; the contents of each of which are herein incorporated by reference in their entireties).

In one embodiment, the vector genome comprises a UBC promoter. The UBC promoter may have a size of 300-350 nucleotides. As a non-limiting example, the UBC promoter is 332 nucleotides.

In one embodiment, the vector genome comprises a GUSB promoter. The GUSB promoter may have a size of 350-400 nucleotides. As a non-limiting example, the GUSB promoter is 378 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a NFL promoter. The NFL promoter may have a size of 600-700 nucleotides. As a non-limiting example, the NFL promoter is 650 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a NFH promoter. The NFH promoter may have a size of 900-950 nucleotides. As a non-limiting example, the NFH promoter is 920 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a scn8a promoter. The scn8a promoter may have a size of 450-500 nucleotides. As a non-limiting example, the scn8a promoter is 470 nucleotides. As a non-limiting example, the construct may be AAV-promoter-CMV/globin intron-hFXN-RBG, where the AAV may be self-complementary and the AAV may be the DJ serotype.

In one embodiment, the vector genome comprises a FXN promoter.

In one embodiment, the vector genome comprises a PGK promoter.

In one embodiment, the vector genome comprises a CBA promoter.

In one embodiment, the vector genome comprises a CMV promoter.

In one embodiment, the vector genome comprises a liver or a skeletal muscle promoter. Non-limiting examples of liver promoters include hAAT and TBG. Non-limiting examples of skeletal muscle promoters include Desmin, MCK and C5-12.

In one embodiment, the expression vector comprises an enhancer element, a promoter and/or a 5′UTR intron. The enhancer may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In one embodiment, the expression vector has an engineered promoter.

### Introns

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in their entirety) such as an intron. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The promoter may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

Introduction into Cells

The modulatory polynucleotides of the invention can be introduced into host cells using any of a variety of approaches. Infection with a viral vector comprising the modulatory polynucleotide can be affected. Examples of suitable viral vectors include replication defective retroviral vectors, adenoviral vectors, adeno-associated vectors and lentiviral vectors.

According to the present invention, viral vectors for use in therapeutics and/or diagnostics comprise a virus that has been distilled or reduced to the minimum components necessary for transduction of a nucleic acid payload or cargo of interest.

In this manner, viral vectors are engineered as vehicles for specific delivery while lacking the deleterious replication and/or integration features found in wild-type virus.

As used herein, a “vector” is any molecule or moiety which transports, transduces or otherwise acts as a carrier of a heterologous molecule such as the modulatory polynucleotides of the invention. A “viral vector” is a vector which comprises one or more polynucleotide regions encoding or comprising payload molecules of interest, e.g., a transgene, a polynucleotide encoding a polypeptide or multi-polypeptide or a modulatory nucleic acid. Viral vectors of the present invention may be produced recombinantly and may be based on adeno-associated virus (AAV) parent or reference sequences. Serotypes which may be useful in the present invention include any of those arising from AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ and AAV-DJ8.

In one embodiment, the serotype which may be useful in the present invention may be AAV-DJ8. The amino acid sequence of AAV-DJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

AAV vectors may also comprise self-complementary AAV vectors (scAAVs). scAAV vectors contain both DNA strands which anneal together to form double stranded DNA. By skipping second strand synthesis, scAAVs allow for rapid expression in the cell.

In one embodiment, the AAV vector used in the present invention is a scAAV.

In one embodiment, the modulatory polynucleotides may be introduced into cells from any relevant species, such as, but not limited to, human, dog, mouse, rat or monkey.

In one embodiment, the modulatory polynucleotides may be introduced into cells which are relevant to the disease to be treated. As a non-limiting example, the disease is ALS and the target cells are motor neurons and astrocytes.

In one embodiment, the modulatory polynucleotides may be introduced into cells which have a high level of endogenous expression of the target sequence.

In another embodiment, the modulatory polynucleotides may be introduced into cells which have a low level of endogenous expression of the target sequence.

In one embodiment, the cells may be those which have a high efficiency of AAV transduction.

In one embodiment, the cells which may be used for in vitro analysis of the modulatory polynucleotides include, but are not limited to, HEK293, HeLa, human primary astrocytes, human astrocyte cell line (U251MG), SH-SY5Y-neurons and human iPSC-derived motor neuron progenitors.

### Target Nucleic Acids

The modulatory polynucleotides of the invention may be targeted to any gene or nucleic acid construct including coding and non-coding genes. Genes (DNA or mRNA) that encode human or primate proteins may be targeted. Further, non-coding genes may also be targeted, e.g., long noncoding RNAs (lncRNA).

Examples of such lncRNA molecules and RNAi constructs designed to target such lncRNA any of which may be targeted by or encoded in the modulatory polynucleotides, respectively are taught in International Publication, WO2012/018881 A2, the contents of which are incorporated herein by reference in their entirety.

In one embodiment, the modulatory polynucleotides of the invention may target any gene known in the art. As a non-limiting example, the gene may be SOD1.

In one embodiment, the modulatory polynucleotide may target a sequence 15-19 nucleotides in length. As a non-limiting example, the target may be any of the sequences described in Table 1. As another non-limiting example, the target may be nucleotides 406-424 of NM_000454.4. As yet another non-limiting example, the target may be nucleotides 645-661 of NM_000454.4.

In one embodiment, the modulatory polynucleotide may target a sequence 21 nucleotides in length. In one aspect, the target may be any 21 mer sequence of NM_000454.4 or any gene known in the art. As a non-limiting example, the target may be nucleotides 521-541 of NM_000454.4. As another non-limiting example, the target may be nucleotides 639-659 of NM_000454.4. As another non-limiting example, the target may be nucleotides 640-660 of NM_000454.4. As another non-limiting example, the target may be nucleotides 645-665 of NM_000454.4. As another non-limiting example, the target may be nucleotides 664-684 of NM_000454.4.

In one embodiment, the modulatory polynucleotide may be designed to target any gene or mRNA in the human genome, e.g., genes associated with CNS disorders such as, but not limited to, Huntington's Disease, ALS and the like.

### Pharmaceutical Compositions

Although the descriptions of pharmaceutical compositions, e.g., those modulatory polynucleotides (including the encoding plasmids or expression vectors, such as viruses, e.g., AAV) comprising a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the viral vector carrying the payload or to the modulatory polynucleotide payload molecule delivered by a viral vector as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered.

### Formulation

The modulatory polynucleotides or viral vectors encoding them can be formulated using one or more excipients to: (1) increase stability; (2) increase cell transfection or transduction; (3) permit the sustained or delayed release; or (4) alter the biodistribution (e.g., target the viral vector to specific tissues or cell types).

Formulations of the present invention can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one payload molecule. As a non-limiting example, the formulations may contain 1, 2, 3, 4 or 5 modulatory polynucleotide payload molecules. In one embodiment the formulation may contain a modulatory polynucleotide payload construct targeting proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three payload construct targeting proteins.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by the United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, modulatory polynucleotide formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present invention may be approved by the US Food and Drug Administration (FDA).

Formulations of viral vectors carrying modulatory polynucleotide disclosed herein may include cations or anions. In one embodiment, the formulations include metal cations such as, but not limited to, Zn2+, Ca2+, Cu2+, Mg+ and combinations thereof. As a non-limiting example, formulations may include polymers and modulatory polynucleotides complexed with a metal cation (See e.g., U.S. Pat. Nos. 6,265,389 and 6,555,525, each of which is herein incorporated by reference in its entirety).

### Administration

The viral vectors comprising modulatory polynucleotides of the present invention may be administered by any route which results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them to cross the blood-brain barrier, vascular barrier, or other epithelial barrier. In one embodiment, a formulation for a route of administration may include at least one inactive ingredient.

### Dosing

The present invention provides methods comprising administering viral vectors and their modulatory polynucleotide payload or complexes in accordance with the invention to a subject in need thereof. Viral vector pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific modulatory polynucleotide payload employed; the duration of the treatment; drugs used in combination or coincidental with the specific compound employed; and like factors well known in the medical arts.

In certain embodiments, viral vector pharmaceutical compositions in accordance with the present invention may be administered at modulatory polynucleotide dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired modulatory polynucleotide dosage may be delivered more than once (e.g., more than one administration in a day). In certain embodiments, the desired modulatory polynucleotide dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in 24 hour period. It may be administered as a single unit dose. In one embodiment, the viral vectors comprising the modulatory polynucleotides of the present invention are administered to a subject in split doses. They may be formulated in buffer only or in a formulation described herein.

In one embodiment, delivery of the compositions in accordance with the present invention to cells comprises a rate of delivery defined by [VG/hour=mL/hour*VG/mL] wherein VG is viral genomes, VG/mL is composition concentration, and mL/hour is rate of prolonged delivery.

In one embodiment, delivery of compositions in accordance with the present invention to cells may comprise a total concentration per subject between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹² 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹² 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In one embodiment, delivery of compositions in accordance with the present invention to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹² 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

In one embodiment, about 10⁵ to 10⁶ viral genome (unit) may be administered per dose.

In one embodiment, delivery of the compositions in accordance with the present invention to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

### Combinations

The viral vectors comprising the modulatory polynucleotide may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Delivery

In one embodiment, the viral vector comprising a modulatory polynucleotide may be administered or delivered using the methods for the delivery of AAV virions described in European Patent Application No. EP1857552, the contents of which are herein incorporated by reference in their entirety.

In one embodiment, the viral vector comprising a modulatory polynucleotide may be administered or delivered using the methods for delivering proteins using AAV vectors described in European Patent Application No. EP2678433, the contents of which are herein incorporated by reference in their entirety.

In one embodiment, the viral vector comprising a modulatory polynucleotide may be administered or delivered using the methods for delivering DNA molecules using AAV vectors described in U.S. Pat. No. 5,858,351, the contents of which are herein incorporated by reference in their entirety.

In one embodiment, the viral vector comprising a modulatory polynucleotide may be administered or delivered using the methods for delivering DNA to the bloodstream described in U.S. Pat. No. 6,211,163, the contents of which are herein incorporated by reference in their entirety.

In one embodiment, the viral vector comprising a modulatory polynucleotide may be administered or delivered using the methods for delivering AAV virions described in U.S. Pat. No. 6,325,998, the contents of which are herein incorporated by reference in their entirety.

In one embodiment, the viral vector comprising a modulatory polynucleotide may be administered or delivered using the methods for delivering DNA to muscle cells described in U.S. Pat. No. 6,335,011, the contents of which are herein incorporated by reference in their entirety.

In one embodiment, the viral vector comprising a modulatory polynucleotide may be administered or delivered using the methods for delivering DNA to muscle cells and tissues described in U.S. Pat. No. 6,610,290, the contents of which are herein incorporated by reference in their entirety.

In one embodiment, the viral vector comprising a modulatory polynucleotide may be administered or delivered using the methods for delivering DNA to muscle cells described in U.S. Pat. No. 7,704,492, the contents of which are herein incorporated by reference in their entirety.

In one embodiment, the viral vector comprising a modulatory polynucleotide may be administered or delivered using the methods for delivering a payload to skeletal muscles described in U.S. Pat. No. 7,112,321, the contents of which are herein incorporated by reference in their entirety.

In one embodiment, the viral vector may be administered or delivered using the methods for delivering a payload to the central nervous system described in U.S. Pat. No. 7,588,757, the contents of which are herein incorporated by reference in their entirety.

In one embodiment, the viral vector comprising a modulatory polynucleotide may be administered or delivered using the methods for delivering a payload described in U.S. Pat. No. 8,283,151, the contents of which are herein incorporated by reference in their entirety.

In one embodiment, the viral vector comprising a modulatory polynucleotide may be administered or delivered using the methods for delivering a payload for the treatment of Alzheimer disease described in U.S. Pat. No. 8,318,687, the contents of which are herein incorporated by reference in their entirety.

In one embodiment, the viral vector comprising a modulatory polynucleotide may be administered or delivered using the methods for delivering a payload described in International Patent Publication No. WO2012144446, the contents of which are herein incorporated by reference in their entirety.

In one embodiment, the viral vector comprising a modulatory polynucleotide may be administered or delivered using the methods for delivering a payload using a glutamic acid decarboxylase (GAD) delivery vector described in International Patent Publication No. WO2001089583, the contents of which are herein incorporated by reference in their entirety.

In one embodiment, the viral vector comprising a modulatory polynucleotide may be administered or delivered using the methods for delivering a payload described in International Patent Publication No. WO2001096587, the contents of which are herein incorporated by reference in their entirety.

In one embodiment, the viral vector comprising a modulatory polynucleotide may be administered or delivered using the methods for delivering a payload to muscle tissue described in International Patent Publication No. WO2002014487, the contents of which are herein incorporated by reference in their entirety.

In one embodiment, the viral vector comprising a modulatory polynucleotide may be administered or delivered using the methods for delivering a payload to neural cells described in International Patent Publication No. WO2012057363, the contents of which are herein incorporated by reference in their entirety.

The pharmaceutical compositions of viral vectors described herein may be characterized by one or more of bioavailability, therapeutic window and/or volume of distribution.

In one embodiment, the viral vectors comprising a modulatory polynucleotide may be formulated. As a non-limiting example the baricity and/or osmolality of the formulation may be optimized to ensure optimal drug distribution in the central nervous system or a region or component of the central nervous system.

In one embodiment, the viral vectors comprising a modulatory polynucleotide may be delivered to a subject via a single route administration.

In one embodiment, the viral vectors comprising a modulatory polynucleotide may be delivered to a subject via a multi-site route of administration. A subject may be administered the viral vectors comprising a modulatory polynucleotide at 2, 3, 4, 5 or more than 5 sites.

In one embodiment, a subject may be administered the viral vectors comprising a modulatory polynucleotide described herein using a bolus infusion.

In one embodiment, a subject may be administered the viral vectors comprising a modulatory polynucleotide described herein using sustained delivery over a period of minutes, hours or days. The infusion rate may be changed depending on the subject, distribution, formulation or another delivery parameter.

In one embodiment, the catheter may be located at more than one site in the spine for multi-site delivery. The viral vectors comprising a modulatory polynucleotide may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, a subject may be analyzed for spinal anatomy and pathology prior to delivery of the viral vectors comprising a modulatory polynucleotide described herein. As a non-limiting example, a subject with scoliosis may have a different dosing regimen and/or catheter location compared to a subject without scoliosis.

In one embodiment, the orientation of the spine subject during delivery of the viral vectors comprising a modulatory polynucleotide may be vertical to the ground.

In another embodiment, the orientation of the spine of the subject during delivery of the viral vectors comprising a modulatory polynucleotide may be horizontal to the ground.

In one embodiment, the spine of the subject may be at an angle as compared to the ground during the delivery of the viral vectors comprising a modulatory polynucleotide subject. The angle of the spine of the subject as compared to the ground may be at least 10, 20, 30, 40, 50, 60, 70, 80, 90, 100, 110, 120, 130, 140, 150 or 180 degrees.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

### Bioavailability

Viral vectors comprising a modulatory polynucleotide of the present invention, when formulated into compositions with delivery/formulation agents or vehicles as described herein, may exhibit increased bioavailability as compared to compositions lacking delivery agents as described herein. As used herein, the term “bioavailability” refers to the systemic availability of a given amount of a particular agent administered to a subject. Bioavailability may be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound may be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Cₘₐₓ values are maximum concentrations of compounds achieved in serum or plasma of a subject following administration of compounds to the subject. Cₘₐₓ values of particular compounds may be measured using methods known to those of ordinary skill in the art. As used herein, the phrases “increasing bioavailability” or “improving the pharmacokinetics,” refer to actions that may increase the systemic availability of a viral vector of the present invention (as measured by AUC, Cₘₐₓ, or Cₘᵢₙ) in a subject. In some embodiments, such actions may comprise co-administration with one or more delivery agents as described herein. In some embodiments, the bioavailability of viral vectors may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Therapeutic Window

Viral vectors comprising a modulatory polynucleotide of the present invention, when formulated with one or more delivery agents as described herein, may exhibit increases in the therapeutic window of compound and/or composition administration as compared to the therapeutic window of viral vectors administered without one or more delivery agents as described herein. As used herein, the term “therapeutic window” refers to the range of plasma concentrations, or the range of levels of therapeutically active substance at the site of action, with a high probability of eliciting a therapeutic effect. In some embodiments, therapeutic windows of viral vectors when administered in a formulation may increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%.

### Volume of Distribution

Viral vectors comprising a modulatory polynucleotide of the present invention, when formulated with one or more delivery agents as described herein, may exhibit an improved volume of distribution (V_{dist}), e.g., reduced or targeted, relative to formulations lacking one or more delivery agents as described herein. V_{dist} relates the amount of an agent in the body to the concentration of the same agent in the blood or plasma. As used herein, the term “volume of distribution” refers to the fluid volume that would be required to contain the total amount of an agent in the body at the same concentration as in the blood or plasma: V_{dist} equals the amount of an agent in the body/concentration of the agent in blood or plasma. For example, for a 10 mg dose of a given agent and a plasma concentration of 10 mg/L, the volume of distribution would be 1 liter. The volume of distribution reflects the extent to which an agent is present in the extravascular tissue. Large volumes of distribution reflect the tendency of agents to bind to the tissue components as compared with plasma proteins. In clinical settings, V_{dist} may be used to determine loading doses to achieve steady state concentrations. In some embodiments, volumes of distribution of viral vector compositions of the present invention when co-administered with one or more delivery agents as described herein may decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Kits and Devices

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

Any of the vectors, constructs, modulatory polynucleotides, polynucleotides or polypeptides of the present invention may be comprised in a kit. In some embodiments, kits may further include reagents and/or instructions for creating and/or synthesizing compounds and/or compositions of the present invention. In some embodiments, kits may also include one or more buffers. In some embodiments, kits of the invention may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquotted. Where there are more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present invention may also typically include means for containing compounds and/or compositions of the present invention, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present invention may be combined with, coated onto or embedded in a device. Devices may include, but are not limited to, dental implants, stents, bone replacements, artificial joints, valves, pacemakers and/or other implantable therapeutic device.

The present invention provides for devices which may incorporate viral vectors that encode one or more modulatory polynucleotide payload molecules. These devices contain in a stable formulation the viral vectors which may be immediately delivered to a subject in need thereof, such as a human patient.

Devices for administration may be employed to deliver the viral vectors comprising a modulatory polynucleotide of the present invention according to single, multi- or split-dosing regimens taught herein.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

The modulatory polynucleotides of the present invention may be used in the treatment, prophylaxis or amelioration of any disease or disorder characterized by aberrant or undesired target expression.

### Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a modulatory polynucleotide of the present invention may be considered biologically active if even a portion of the polynucleotides is biologically active or mimics an activity considered biologically relevant.

Induced pluripotent stem cells: As used herein, “induced pluripotent stem cells” are cells that may be induced to form any of several distinct cell types.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Controlled Release: As used herein, the term “controlled release” refers to a pharmaceutical composition or compound release profile that conforms to a particular pattern of release to effect a therapeutic outcome.

Cyclic or Cyclized: As used herein, the term “cyclic” refers to the presence of a continuous loop. Cyclic molecules need not be circular, only joined to form an unbroken chain of subunits.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a modulatory polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Diastereomer: As used herein, the term “diastereomer,” means stereoisomers that are not mirror images of one another and are non-superimposable on one another.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Enantiomer: As used herein, the term “enantiomer” means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Exosome: As used herein, “exosome” is a vesicle secreted by mammalian cells or a complex involved in RNA degradation.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one modulatory polynucleotide and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

Isomer: As used herein, the term “isomer” means any tautomer, stereoisomer, enantiomer, or diastereomer of any compound of the invention. It is recognized that the compounds of the invention can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form modulatory polynucleotide multimers (e.g., through linkage of two or more modulatory polynucleotides molecules) or modulatory polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the invention. Molecules may be modified in many ways including chemically, structurally, and functionally.

Naturally occurring: As used herein, “naturally occurring” means existing in nature without artificial aid.

Neutralizing antibody: As used herein, a “neutralizing antibody” refers to an antibody which binds to its antigen and defends a cell from an antigen or infectious agent by neutralizing or abolishing any biological activity it has.

Non-human vertebrate: As used herein, a “non human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Optionally substituted: Herein a phrase of the form “optionally substituted X” (e.g., optionally substituted alkyl) is intended to be equivalent to “X, wherein X is optionally substituted” (e.g., “alkyl, wherein the alkyl is optionally substituted”). It is not intended to mean that the feature “X” (e.g. alkyl) per se is optional.

Peptide: As used herein, “peptide” is less than or equal to 50 amino acids long, e.g., about 5, 10, 15, 20, 25, 30, 35, 40, 45, or 50 amino acids long.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrates, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety. In some embodiments, the pri-miRs of the invention may be prodrugs of the pre-miRs. Likewise either pri- or pre-miRs may be prodrugs of the artificial miRs which are processed from them.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Stereoisomer: As used herein, the term “stereoisomer” refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Design of Modulatory Polynucleotides (Artificial Pri- or Pre-microRNAs)

Artificial pri- or pre-microRNAs are designed as shRNA or stem loop structures encoding an artificial miR (or artificial siRNA) having at least one strand that can at least partially hybridize with a target nucleic acid, e.g., RNA or DNA and one or more of the following features (a) UG motif at the base of basal stem, (b) a UGUG motif at the 5′ end of the miRNA loop, (c) Uridine at the 5′ end of guide strand, (d) a loop structure derived from a canonical microRNA such as miR-22 (e) a CNNC at the 3′ flanking sequence, (f) flanking regions from a canonical microRNA such as let-7b and/or (g) one or more bulges and mismatches as between the passenger and guide strand.

Once designed, the sequence is engineered or synthesized or inserted in a plasmid or vector and administered to a cell or organism. Suitable plasmids or vectors are any which transduce or transfect the target cell.

Adeno-associated viral vectors (AAV), viral particles or entire viruses may be used.

Administration results in the processing of the modulatory polynucleotide to generate the artificial microRNA which alters expression levels of the target nucleic acid.

Effective knockdown of a target may be determined by methods in the art and will show little if any off-target effects.

Effective passenger-guide strand duplexes of the modulatory polynucleotides, e.g., pri- or pre-microRNAs demonstrate greater than 95% guide to passenger strand ratio when processing is measured.

### Example 2. Passenger-Guide Strand Optimization

In order to achieve target knockdown or modulation of target expression which is specific and potent, the passenger and guide strands that will form the duplex stem of the stem-loop structure of the pri- or pre-microRNA of the invention may be optimized separately, for example as siRNA (small interfering RNAs).

siRNAs are designed against a target nucleic acid of choice as canonical siRNAs having a 19 base pair central duplex with a 3′ dinucleotide overhang on the 3′ end of the strands of the duplex and where the antisense strand has perfect complementarity to the target nucleic acid over the 19 nucleotide region.

Alternatively, siRNAs are designed whereby the sense strand (passenger strand) comprises less than 19 nucleotide identity to the target nucleic acid.

Modifications to the sense-antisense (passenger-guide) strand duplex base pairing is made to introduce bulges or mismatches. Insertions or deletions or mismatches may be incorporated at the 5′ or 3′ terminus of the sense strand and these insertions or deletions may or may not be mirrored on the guide strand.

The resulting siRNA are tested by standard methods known in the art for target knockdown and other relevant physiologic and pharmacokinetic properties and for degree of off-target effects.

siRNA exhibiting sufficient target knockdown with few off target effects are then engineered, either with or without further modifications, as the passenger and guide strands of the pri- or pre-microRNAs of the invention.

### Example 3. Passenger-Guide Strand Design for SOD1

In engineering optimal passenger and guide strands for the pri- and/or pre-microRNAs of the invention, a series of 19-mer sense strand (passenger strand) sequences were chosen from the sequence of superoxide dismutase 1 (SOD1; GenBank Reference NM_000454.4). The sequence of the SOD1 mRNA (shown as DNA) is

(SEQ ID NO: 15)
GTTTGGGGCCAGAGTGGGCGAGGCGCGGAGGTCTGGCCTATAAAGTAGTC
GCGGAGACGGGGTGCTGGTTTGCGTCGTAGTCTCCTGCAGCGTCTGGGGT
TTCCGTTGCAGTCCTCGGAACCAGGACCTCGGCGTGGCCTAGCGAGTTAT
GGCGACGAAGGCCGTGTGCGTGCTGAAGGGCGACGGCCCAGTGCAGGGCA
TCATCAATTTCGAGCAGAAGGAAAGTAATGGACCAGTGAAGGTGTGGGGA
AGCATTAAAGGACTGACTGAAGGCCTGCATGGATTCCATGTTCATGAGTT
TGGAGATAATACAGCAGGCTGTACCAGTGCAGGTCCTCACTTTAATCCTC
TATCCAGAAAACACGGTGGGCCAAAGGATGAAGAGAGGCATGTTGGAGAC
TTGGGCAATGTGACTGCTGACAAAGATGGTGTGGCCGATGTGTCTATTGA
AGATTCTGTGATCTCACTCTCAGGAGACCATTGCATCATTGGCCGCACAC
TGGTGGTCCATGAAAAAGCAGATGACTTGGGCAAAGGTGGAAATGAAGAA
AGTACAAAGACAGGAAACGCTGGAAGTCGTTTGGCTTGTGGTGTAATTGG
GATCGCCCAATAAACATTCCCTTGGATGTAGTCTGAGGCCCCTTAACTCA
TCTGTTATCCTGCTAGCTGTAGAAATGTATCCTGATAAACATTAAACACT
GTAATCTTAAAAGTGTAATTGTGTGACTTTTTCAGAGTTGCTTTAAAGTA
CCTGTAGTGAGAAACTGATTTATGATCACTTGGAAGATTTGTATAGTTTT
ATAAAACTCAGTTAAAATGTCTGTTTCAATGACCTGTATTTTGCCAGACT
TAAATCACAGATGGGTATTAAACTTGTCAGAATTTCTTTGTCATTCAAGC
CTGTGAATAAAAACCCTGTATGGCACTTATTATGAGGCTATTAAAAGAAT
CCAAATTCAAACTAAAAAAAAAAAAAAAAAA.

The 19mers, along with the 5′ most position of the sense strand are shown in Table 4 along with the antisense strand which is the reverse complement of the sense strand.

The 19mers served as the core starting sequences for the design of the siRNA to be tested.

TABLE 4
SOD1 19mers

| Start Position | | | | |
|---|---|---|---|---|
| of sense | | | | |
| strand in | Sense Strand, e.g., | SEQ | Antisense Strand, e.g., | SEQ |
| NM_000454.4 | Passenger Strand (5′-3′) | ID NO | Guide Strand (5′-3′) | ID NO |
| 26 | CGGAGGUCUGGCCUAUAAA | 16 | UUUAUAGGCCAGACCUCCG | 17 |
| 27 | GGAGGUCUGGCCUAUAAAG | 18 | CUUUAUAGGCCAGACCUCC | 19 |
| 28 | GAGGUCUGGCCUAUAAAGU | 20 | ACUUUAUAGGCCAGACCUC | 21 |
| 29 | AGGUCUGGCCUAUAAAGUA | 22 | UACUUUAUAGGCCAGACCU | 23 |
| 30 | GGUCUGGCCUAUAAAGUAG | 24 | CUACUUUAUAGGCCAGACC | 25 |
| 32 | UCUGGCCUAUAAAGUAGUC | 26 | GACUACUUUAUAGGCCAGA | 27 |
| 33 | CUGGCCUAUAAAGUAGUCG | 28 | CGACUACUUUAUAGGCCAG | 29 |
| 34 | UGGCCUAUAAAGUAGUCGC | 30 | GCGACUACUUUAUAGGCCA | 31 |
| 35 | GGCCUAUAAAGUAGUCGCG | 32 | CGCGACUACUUUAUAGGCC | 33 |
| 36 | GCCUAUAAAGUAGUCGCGG | 34 | CCGCGACUACUUUAUAGGC | 35 |
| 37 | CCUAUAAAGUAGUCGCGGA | 36 | UCCGCGACUACUUUAUAGG | 37 |
| 74 | GUCGUAGUCUCCUGCAGCG | 38 | CGCUGCAGGAGACUACGAC | 39 |
| 76 | CGUAGUCUCCUGCAGCGUC | 40 | GACGCUGCAGGAGACUACG | 41 |
| 77 | GUAGUCUCCUGCAGCGUCU | 42 | AGACGCUGCAGGAGACUAC | 43 |
| 78 | UAGUCUCCUGCAGCGUCUG | 44 | CAGACGCUGCAGGAGACUA | 45 |
| 149 | AUGGCGACGAAGGCCGUGU | 46 | ACACGGCCUUCGUCGCCAU | 47 |
| 153 | CGACGAAGGCCGUGUGCGU | 48 | ACGCACACGGCCUUCGUCG | 49 |
| 157 | GAAGGCCGUGUGCGUGCUG | 50 | CAGCACGCACACGGCCUUC | 51 |
| 160 | GGCCGUGUGCGUGCUGAAG | 52 | CUUCAGCACGCACACGGCC | 53 |
| 177 | AGGGCGACGGCCCAGUGCA | 54 | UGCACUGGGCCGUCGCCCU | 55 |
| 192 | UGCAGGGCAUCAUCAAUUU | 56 | AAAUUGAUGAUGCCCUGCA | 57 |
| 193 | GCAGGGCAUCAUCAAUUUC | 58 | GAAAUUGAUGAUGCCCUGC | 59 |
| 195 | AGGGCAUCAUCAAUUUCGA | 60 | UCGAAAUUGAUGAUGCCCU | 61 |
| 196 | GGGCAUCAUCAAUUUCGAG | 62 | CUCGAAAUUGAUGAUGCCC | 63 |
| 197 | GGCAUCAUCAAUUUCGAGC | 64 | GCUCGAAAUUGAUGAUGCC | 65 |
| 198 | GCAUCAUCAAUUUCGAGCA | 66 | UGCUCGAAAUUGAUGAUGC | 67 |
| 199 | CAUCAUCAAUUUCGAGCAG | 68 | CUGCUCGAAAUUGAUGAUG | 69 |
| 206 | AAUUUCGAGCAGAAGGAAA | 70 | UUUCCUUCUGCUCGAAAUU | 71 |
| 209 | UUCGAGCAGAAGGAAAGUA | 72 | UACUUUCCUUCUGCUCGAA | 73 |
| 210 | UCGAGCAGAAGGAAAGUAA | 74 | UUACUUUCCUUCUGCUCGA | 75 |
| 239 | AAGGUGUGGGGAAGCAUUA | 76 | UAAUGCUUCCCCACACCUU | 77 |
| 241 | GGUGUGGGGAAGCAUUAAA | 78 | UUUAAUGCUUCCCCACACC | 79 |
| 261 | GACUGACUGAAGGCCUGCA | 80 | UGCAGGCCUUCAGUCAGUC | 81 |
| 263 | CUGACUGAAGGCCUGCAUG | 82 | CAUGCAGGCCUUCAGUCAG | 83 |
| 264 | UGACUGAAGGCCUGCAUGG | 84 | CCAUGCAGGCCUUCAGUCA | 85 |
| 268 | UGAAGGCCUGCAUGGAUUC | 86 | GAAUCCAUGCAGGCCUUCA | 87 |
| 269 | GAAGGCCUGCAUGGAUUCC | 88 | GGAAUCCAUGCAGGCCUUC | 89 |
| 276 | UGCAUGGAUUCCAUGUUCA | 90 | UGAACAUGGAAUCCAUGCA | 91 |
| 278 | CAUGGAUUCCAUGUUCAUG | 92 | CAUGAACAUGGAAUCCAUG | 93 |
| 281 | GGAUUCCAUGUUCAUGAGU | 94 | ACUCAUGAACAUGGAAUCC | 95 |
| 284 | UUCCAUGUUCAUGAGUUUG | 96 | CAAACUCAUGAACAUGGAA | 97 |
| 290 | GUUCAUGAGUUUGGAGAUA | 98 | UAUCUCCAAACUCAUGAAC | 99 |
| 291 | UUCAUGAGUUUGGAGAUAA | 100 | UUAUCUCCAAACUCAUGAA | 101 |
| 295 | UGAGUUUGGAGAUAAUACA | 102 | UGUAUUAUCUCCAAACUCA | 103 |
| 296 | GAGUUUGGAGAUAAUACAG | 104 | CUGUAUUAUCUCCAAACUC | 105 |
| 316 | AGGCUGUACCAGUGCAGGU | 106 | ACCUGCACUGGUACAGCCU | 107 |
| 317 | GGCUGUACCAGUGCAGGUC | 108 | GACCUGCACUGGUACAGCC | 109 |
| 329 | GCAGGUCCUCACUUUAAUC | 110 | GAUUAAAGUGAGGACCUGC | 111 |
| 330 | CAGGUCCUCACUUUAAUCC | 112 | GGAUUAAAGUGAGGACCUG | 113 |
| 337 | UCACUUUAAUCCUCUAUCC | 114 | GGAUAGAGGAUUAAAGUGA | 115 |
| 350 | CUAUCCAGAAAACACGGUG | 116 | CACCGUGUUUUCUGGAUAG | 117 |
| 351 | UAUCCAGAAAACACGGUGG | 118 | CCACCGUGUUUUCUGGAUA | 119 |
| 352 | AUCCAGAAAACACGGUGGG | 120 | CCCACCGUGUUUUCUGGAU | 121 |
| 354 | CCAGAAAACACGGUGGGCC | 122 | GGCCCACCGUGUUUUCUGG | 123 |
| 357 | GAAAACACGGUGGGCCAAA | 124 | UUUGGCCCACCGUGUUUUC | 125 |
| 358 | AAAACACGGUGGGCCAAAG | 126 | CUUUGGCCCACCGUGUUUU | 127 |
| 364 | CGGUGGGCCAAAGGAUGAA | 128 | UUCAUCCUUUGGCCCACCG | 129 |
| 375 | AGGAUGAAGAGAGGCAUGU | 130 | ACAUGCCUCUCUUCAUCCU | 131 |
| 378 | AUGAAGAGAGGCAUGUUGG | 132 | CCAACAUGCCUCUCUUCAU | 133 |
| 383 | GAGAGGCAUGUUGGAGACU | 134 | AGUCUCCAACAUGCCUCUC | 135 |
| 384 | AGAGGCAUGUUGGAGACUU | 136 | AAGUCUCCAACAUGCCUCU | 137 |
| 390 | AUGUUGGAGACUUGGGCAA | 138 | UUGCCCAAGUCUCCAACAU | 139 |
| 392 | GUUGGAGACUUGGGCAAUG | 140 | CAUUGCCCAAGUCUCCAAC | 141 |
| 395 | GGAGACUUGGGCAAUGUGA | 142 | UCACAUUGCCCAAGUCUCC | 143 |
| 404 | GGCAAUGUGACUGCUGACA | 144 | UGUCAGCAGUCACAUUGCC | 145 |
| 406 | CAAUGUGACUGCUGACAAA | 146 | UUUGUCAGCAGUCACAUUG | 147 |
| 417 | CUGACAAAGAUGGUGUGGC | 148 | GCCACACCAUCUUUGUCAG | 149 |
| 418 | UGACAAAGAUGGUGUGGCC | 150 | GGCCACACCAUCUUUGUCA | 151 |
| 469 | CUCAGGAGACCAUUGCAUC | 152 | GAUGCAAUGGUCUCCUGAG | 153 |
| 470 | UCAGGAGACCAUUGCAUCA | 154 | UGAUGCAAUGGUCUCCUGA | 155 |
| 475 | AGACCAUUGCAUCAUUGGC | 156 | GCCAAUGAUGCAAUGGUCU | 157 |
| 476 | GACCAUUGCAUCAUUGGCC | 158 | GGCCAAUGAUGCAAUGGUC | 159 |
| 480 | AUUGCAUCAUUGGCCGCAC | 160 | GUGCGGCCAAUGAUGCAAU | 161 |
| 487 | CAUUGGCCGCACACUGGUG | 162 | CACCAGUGUGCGGCCAAUG | 163 |
| 494 | CGCACACUGGUGGUCCAUG | 164 | CAUGGACCACCAGUGUGCG | 165 |
| 496 | CACACUGGUGGUCCAUGAA | 166 | UUCAUGGACCACCAGUGUG | 167 |
| 497 | ACACUGGUGGUCCAUGAAA | 168 | UUUCAUGGACCACCAGUGU | 169 |
| 501 | UGGUGGUCCAUGAAAAAGC | 170 | GCUUUUUCAUGGACCACCA | 171 |
| 504 | UGGUCCAUGAAAAAGCAGA | 172 | UCUGCUUUUUCAUGGACCA | 173 |
| 515 | AAAGCAGAUGACUUGGGCA | 174 | UGCCCAAGUCAUCUGCUUU | 175 |
| 518 | GCAGAUGACUUGGGCAAAG | 176 | CUUUGCCCAAGUCAUCUGC | 177 |
| 522 | AUGACUUGGGCAAAGGUGG | 178 | CCACCUUUGCCCAAGUCAU | 179 |
| 523 | UGACUUGGGCAAAGGUGGA | 180 | UCCACCUUUGCCCAAGUCA | 181 |
| 524 | GACUUGGGCAAAGGUGGAA | 182 | UUCCACCUUUGCCCAAGUC | 183 |
| 552 | GUACAAAGACAGGAAACGC | 184 | GCGUUUCCUGUCUUUGUAC | 185 |
| 554 | ACAAAGACAGGAAACGCUG | 186 | CAGCGUUUCCUGUCUUUGU | 187 |
| 555 | CAAAGACAGGAAACGCUGG | 188 | CCAGCGUUUCCUGUCUUUG | 189 |
| 562 | AGGAAACGCUGGAAGUCGU | 190 | ACGACUUCCAGCGUUUCCU | 191 |
| 576 | GUCGUUUGGCUUGUGGUGU | 192 | ACACCACAAGCCAAACGAC | 193 |
| 577 | UCGUUUGGCUUGUGGUGUA | 194 | UACACCACAAGCCAAACGA | 195 |
| 578 | CGUUUGGCUUGUGGUGUAA | 196 | UUACACCACAAGCCAAACG | 197 |
| 579 | GUUUGGCUUGUGGUGUAAU | 198 | AUUACACCACAAGCCAAAC | 199 |
| 581 | UUGGCUUGUGGUGUAAUUG | 200 | CAAUUACACCACAAGCCAA | 201 |
| 583 | GGCUUGUGGUGUAAUUGGG | 202 | CCCAAUUACACCACAAGCC | 203 |
| 584 | GCUUGUGGUGUAAUUGGGA | 204 | UCCCAAUUACACCACAAGC | 205 |
| 585 | CUUGUGGUGUAAUUGGGAU | 206 | AUCCCAAUUACACCACAAG | 207 |
| 587 | UGUGGUGUAAUUGGGAUCG | 208 | CGAUCCCAAUUACACCACA | 209 |
| 588 | GUGGUGUAAUUGGGAUCGC | 210 | GCGAUCCCAAUUACACCAC | 211 |
| 589 | UGGUGUAAUUGGGAUCGCC | 212 | GGCGAUCCCAAUUACACCA | 213 |
| 593 | GUAAUUGGGAUCGCCCAAU | 214 | AUUGGGCGAUCCCAAUUAC | 215 |
| 594 | UAAUUGGGAUCGCCCAAUA | 216 | UAUUGGGCGAUCCCAAUUA | 217 |
| 595 | AAUUGGGAUCGCCCAAUAA | 218 | UUAUUGGGCGAUCCCAAUU | 219 |
| 596 | AUUGGGAUCGCCCAAUAAA | 220 | UUUAUUGGGCGAUCCCAAU | 221 |
| 597 | UUGGGAUCGCCCAAUAAAC | 222 | GUUUAUUGGGCGAUCCCAA | 223 |
| 598 | UGGGAUCGCCCAAUAAACA | 224 | UGUUUAUUGGGCGAUCCCA | 225 |
| 599 | GGGAUCGCCCAAUAAACAU | 226 | AUGUUUAUUGGGCGAUCCC | 227 |
| 602 | AUCGCCCAAUAAACAUUCC | 228 | GGAAUGUUUAUUGGGCGAU | 229 |
| 607 | CCAAUAAACAUUCCCUUGG | 230 | CCAAGGGAAUGUUUAUUGG | 231 |
| 608 | CAAUAAACAUUCCCUUGGA | 232 | UCCAAGGGAAUGUUUAUUG | 233 |
| 609 | AAUAAACAUUCCCUUGGAU | 234 | AUCCAAGGGAAUGUUUAUU | 235 |
| 610 | AUAAACAUUCCCUUGGAUG | 236 | CAUCCAAGGGAAUGUUUAU | 237 |
| 611 | UAAACAUUCCCUUGGAUGU | 238 | ACAUCCAAGGGAAUGUUUA | 239 |
| 612 | AAACAUUCCCUUGGAUGUA | 240 | UACAUCCAAGGGAAUGUUU | 241 |
| 613 | AACAUUCCCUUGGAUGUAG | 242 | CUACAUCCAAGGGAAUGUU | 243 |
| 616 | AUUCCCUUGGAUGUAGUCU | 244 | AGACUACAUCCAAGGGAAU | 245 |
| 621 | CUUGGAUGUAGUCUGAGGC | 246 | GCCUCAGACUACAUCCAAG | 247 |
| 633 | CUGAGGCCCCUUAACUCAU | 248 | AUGAGUUAAGGGGCCUCAG | 249 |
| 635 | GAGGCCCCUUAACUCAUCU | 250 | AGAUGAGUUAAGGGGCCUC | 251 |
| 636 | AGGCCCCUUAACUCAUCUG | 252 | CAGAUGAGUUAAGGGGCCU | 253 |
| 639 | CCCCUUAACUCAUCUGUUA | 254 | UAACAGAUGAGUUAAGGGG | 255 |
| 640 | CCCUUAACUCAUCUGUUAU | 256 | AUAACAGAUGAGUUAAGGG | 257 |
| 641 | CCUUAACUCAUCUGUUAUC | 258 | GAUAACAGAUGAGUUAAGG | 259 |
| 642 | CUUAACUCAUCUGUUAUCC | 260 | GGAUAACAGAUGAGUUAAG | 261 |
| 643 | UUAACUCAUCUGUUAUCCU | 262 | AGGAUAACAGAUGAGUUAA | 263 |
| 644 | UAACUCAUCUGUUAUCCUG | 264 | CAGGAUAACAGAUGAGUUA | 265 |
| 645 | AACUCAUCUGUUAUCCUGC | 266 | GCAGGAUAACAGAUGAGUU | 267 |
| 654 | GUUAUCCUGCUAGCUGUAG | 268 | CUACAGCUAGCAGGAUAAC | 269 |
| 660 | CUGCUAGCUGUAGAAAUGU | 270 | ACAUUUCUACAGCUAGCAG | 271 |
| 661 | UGCUAGCUGUAGAAAUGUA | 272 | UACAUUUCUACAGCUAGCA | 273 |
| 666 | GCUGUAGAAAUGUAUCCUG | 274 | CAGGAUACAUUUCUACAGC | 275 |
| 667 | CUGUAGAAAUGUAUCCUGA | 276 | UCAGGAUACAUUUCUACAG | 277 |
| 668 | UGUAGAAAUGUAUCCUGAU | 278 | AUCAGGAUACAUUUCUACA | 279 |
| 669 | GUAGAAAUGUAUCCUGAUA | 280 | UAUCAGGAUACAUUUCUAC | 281 |
| 673 | AAAUGUAUCCUGAUAAACA | 282 | UGUUUAUCAGGAUACAUUU | 283 |
| 677 | GUAUCCUGAUAAACAUUAA | 284 | UUAAUGUUUAUCAGGAUAC | 285 |
| 692 | UUAAACACUGUAAUCUUAA | 286 | UUAAGAUUACAGUGUUUAA | 287 |
| 698 | ACUGUAAUCUUAAAAGUGU | 288 | ACACUUUUAAGAUUACAGU | 289 |
| 699 | CUGUAAUCUUAAAAGUGUA | 290 | UACACUUUUAAGAUUACAG | 291 |
| 700 | UGUAAUCUUAAAAGUGUAA | 292 | UUACACUUUUAAGAUUACA | 293 |
| 701 | GUAAUCUUAAAAGUGUAAU | 294 | AUUACACUUUUAAGAUUAC | 295 |
| 706 | CUUAAAAGUGUAAUUGUGU | 296 | ACACAAUUACACUUUUAAG | 297 |
| 749 | UACCUGUAGUGAGAAACUG | 298 | CAGUUUCUCACUACAGGUA | 299 |
| 770 | UUAUGAUCACUUGGAAGAU | 300 | AUCUUCCAAGUGAUCAUAA | 301 |
| 772 | AUGAUCACUUGGAAGAUUU | 302 | AAAUCUUCCAAGUGAUCAU | 303 |
| 775 | AUCACUUGGAAGAUUUGUA | 304 | UACAAAUCUUCCAAGUGAU | 305 |
| 781 | UGGAAGAUUUGUAUAGUUU | 306 | AAACUAUACAAAUCUUCCA | 307 |
| 800 | UAUAAAACUCAGUUAAAAU | 308 | AUUUUAACUGAGUUUUAUA | 309 |
| 804 | AAACUCAGUUAAAAUGUCU | 310 | AGACAUUUUAACUGAGUUU | 311 |
| 819 | GUCUGUUUCAAUGACCUGU | 312 | ACAGGUCAUUGAAACAGAC | 313 |
| 829 | AUGACCUGUAUUUUGCCAG | 314 | CUGGCAAAAUACAGGUCAU | 315 |
| 832 | ACCUGUAUUUUGCCAGACU | 316 | AGUCUGGCAAAAUACAGGU | 317 |
| 833 | CCUGUAUUUUGCCAGACUU | 318 | AAGUCUGGCAAAAUACAGG | 319 |
| 851 | UAAAUCACAGAUGGGUAUU | 320 | AAUACCCAUCUGUGAUUUA | 321 |
| 854 | AUCACAGAUGGGUAUUAAA | 322 | UUUAAUACCCAUCUGUGAU | 323 |
| 855 | UCACAGAUGGGUAUUAAAC | 324 | GUUUAAUACCCAUCUGUGA | 325 |
| 857 | ACAGAUGGGUAUUAAACUU | 326 | AAGUUUAAUACCCAUCUGU | 327 |
| 858 | CAGAUGGGUAUUAAACUUG | 328 | CAAGUUUAAUACCCAUCUG | 329 |
| 859 | AGAUGGGUAUUAAACUUGU | 330 | ACAAGUUUAAUACCCAUCU | 331 |
| 861 | AUGGGUAUUAAACUUGUCA | 332 | UGACAAGUUUAAUACCCAU | 333 |
| 869 | UAAACUUGUCAGAAUUUCU | 334 | AGAAAUUCUGACAAGUUUA | 335 |
| 891 | UCAUUCAAGCCUGUGAAUA | 336 | UAUUCACAGGCUUGAAUGA | 337 |
| 892 | CAUUCAAGCCUGUGAAUAA | 338 | UUAUUCACAGGCUUGAAUG | 339 |
| 906 | AAUAAAAACCCUGUAUGGC | 340 | GCCAUACAGGGUUUUUAUU | 341 |
| 907 | AUAAAAACCCUGUAUGGCA | 342 | UGCCAUACAGGGUUUUUAU | 343 |
| 912 | AACCCUGUAUGGCACUUAU | 344 | AUAAGUGCCAUACAGGGUU | 345 |
| 913 | ACCCUGUAUGGCACUUAUU | 346 | AAUAAGUGCCAUACAGGGU | 347 |
| 934 | GAGGCUAUUAAAAGAAUCC | 348 | GGAUUCUUUUAAUAGCCUC | 349 |
| 944 | AAAGAAUCCAAAUUCAAAC | 350 | GUUUGAAUUUGGAUUCUUU | 351 |
| 947 | GAAUCCAAAUUCAAACUAA | 352 | UUAGUUUGAAUUUGGAUUC | 353 |

The core starting sense-antisense pairs of Table 4 above were then engineered as duplex siRNA. In doing so the 3′ most nucleotide of the sense strand was, in all cases, changed to a cytidine (C) nucleotide leaving then only 18 nucleotides with identity to the target.

Then a dinucleotide terminus at the 3′ end of each of the sense and antisense strands was added producing the duplexes of Table 5.

TABLE 5
siRNA duplexes to SOD1

| | duplex | | sense strand | SEQ | | antisense strand | SEQ |
|---|---|---|---|---|---|---|---|
| Start | ID | SS ID | sequence (5′-3′) | ID NO | AS ID | sequence (5′-3′) | ID NO |
| 26 | D-2741 | 7414 | CGGAGGUCUGGCCUAU | 354 | 7415 | UUUAUAGGCCAGACCU | 355 |
| | | | AACdTdT | | | CCGdTdT | |
| 27 | D-2742 | 7416 | GGAGGUCUGGCCUAUA | 356 | 7417 | UUUUAUAGGCCAGACC | 357 |
| | | | AACdTdT | | | UCCdTdT | |
| 28 | D-2743 | 7418 | GAGGUCUGGCCUAUAA | 358 | 7419 | UCUUUAUAGGCCAGAC | 359 |
| | | | AGCdTdT | | | CUCdTdT | |
| 29 | D-2744 | 7420 | AGGUCUGGCCUAUAAA | 360 | 7421 | UACUUUAUAGGCCAGA | 361 |
| | | | GUCdTdT | | | CCUdTdT | |
| 30 | D-2745 | 7422 | GGUCUGGCCUAUAAAG | 362 | 7423 | UUACUUUAUAGGCCAG | 363 |
| | | | UACdTdT | | | ACCdTdT | |
| 32 | D-2746 | 7424 | UCUGGCCUAUAAAGUA | 364 | 7425 | UACUACUUUAUAGGCC | 365 |
| | | | GUCdTdT | | | AGAdTdT | |
| 33 | D-2747 | 7426 | CUGGCCUAUAAAGUAG | 366 | 7427 | UGACUACUUUAUAGGC | 367 |
| | | | UCCdTdT | | | CAGdTdT | |
| 34 | D-2748 | 7428 | UGGCCUAUAAAGUAGU | 368 | 7429 | UCGACUACUUUAUAGG | 369 |
| | | | CGCdTdT | | | CCAdTdT | |
| 35 | D-2749 | 7430 | GGCCUAUAAAGUAGUC | 370 | 7431 | UGCGACUACUUUAUAG | 371 |
| | | | GCCdTdT | | | GCCdTdT | |
| 36 | D-2750 | 7432 | GCCUAUAAAGUAGUCG | 372 | 7433 | UCGCGACUACUUUAUA | 373 |
| | | | CGCdTdT | | | GGCdTdT | |
| 37 | D-2751 | 7434 | CCUAUAAAGUAGUCGC | 374 | 7435 | UCCGCGACUACUUUAU | 375 |
| | | | GGCdTdT | | | AGGdTdT | |
| 74 | D-2752 | 7436 | GUCGUAGUCUCCUGCA | 376 | 7437 | UGCUGCAGGAGACUAC | 377 |
| | | | GCCdTdT | | | GACdTdT | |
| 76 | D-2753 | 7438 | CGUAGUCUCCUGCAGC | 378 | 7439 | UACGCUGCAGGAGACU | 379 |
| | | | GUCdTdT | | | ACGdTdT | |
| 77 | D-2754 | 7440 | GUAGUCUCCUGCAGCG | 380 | 7441 | UGACGCUGCAGGAGAC | 381 |
| | | | UCCdTdT | | | UACdTdT | |
| 78 | D-2755 | 7442 | UAGUCUCCUGCAGCGU | 382 | 7443 | UAGACGCUGCAGGAGA | 383 |
| | | | CUCdTdT | | | CUAdTdT | |
| 149 | D-2756 | 7444 | AUGGCGACGAAGGCCG | 384 | 7445 | UCACGGCCUUCGUCGC | 385 |
| | | | UGCdTdT | | | CAUdTdT | |
| 153 | D-2757 | 7446 | CGACGAAGGCCGUGUG | 386 | 7447 | UCGCACACGGCCUUCG | 387 |
| | | | CGCdTdT | | | UCGdTdT | |
| 157 | D-2758 | 7448 | GAAGGCCGUGUGCGUG | 388 | 7449 | UAGCACGCACACGGCC | 389 |
| | | | CUCdTdT | | | UUCdTdT | |
| 160 | D-2759 | 7450 | GGCCGUGUGCGUGCUG | 390 | 7451 | UUUCAGCACGCACACG | 391 |
| | | | AACdTdT | | | GCCdTdT | |
| 177 | D-2760 | 7452 | AGGGCGACGGCCCAGU | 392 | 7453 | UGCACUGGGCCGUCGC | 393 |
| | | | GCCdTdT | | | CCUdTdT | |
| 192 | D-2761 | 7454 | UGCAGGGCAUCAUCAA | 394 | 7455 | UAAUUGAUGAUGCCCU | 395 |
| | | | UUCdTdT | | | GCAdTdT | |
| 193 | D-2762 | 7456 | GCAGGGCAUCAUCAAU | 396 | 7457 | UAAAUUGAUGAUGCCC | 397 |
| | | | UUCdTdT | | | UGCdTdT | |
| 195 | D-2763 | 7458 | AGGGCAUCAUCAAUUU | 398 | 7459 | UCGAAAUUGAUGAUGC | 399 |
| | | | CGCdTdT | | | CCUdTdT | |
| 196 | D-2764 | 7460 | GGGCAUCAUCAAUUUC | 400 | 7461 | UUCGAAAUUGAUGAUG | 401 |
| | | | GACdTdT | | | CCCdTdT | |
| 197 | D-2765 | 7462 | GGCAUCAUCAAUUUCG | 402 | 7463 | UCUCGAAAUUGAUGAU | 403 |
| | | | AGCdTdT | | | GCCdTdT | |
| 198 | D-2766 | 7464 | GCAUCAUCAAUUUCGA | 404 | 7465 | UGCUCGAAAUUGAUGA | 405 |
| | | | GCCdTdT | | | UGCdTdT | |
| 199 | D-2767 | 7466 | CAUCAUCAAUUUCGAG | 406 | 7467 | UUGCUCGAAAUUGAUG | 407 |
| | | | CACdTdT | | | AUGdTdT | |
| 206 | D-2768 | 7468 | AAUUUCGAGCAGAAGG | 408 | 7469 | UUUCCUUCUGCUCGAA | 409 |
| | | | AACdTdT | | | AUUdTdT | |
| 209 | D-2769 | 7470 | UUCGAGCAGAAGGAAA | 410 | 7471 | UACUUUCCUUCUGCUC | 411 |
| | | | GUCdTdT | | | GAAdTdT | |
| 210 | D-2770 | 7472 | UCGAGCAGAAGGAAAG | 412 | 7473 | UUACUUUCCUUCUGCU | 413 |
| | | | UACdTdT | | | CGAdTdT | |
| 239 | D-2771 | 7474 | AAGGUGUGGGGAAGCA | 414 | 7475 | UAAUGCUUCCCCACAC | 415 |
| | | | UUCdTdT | | | CUUdTdT | |
| 241 | D-2772 | 7476 | GGUGUGGGGAAGCAUU | 416 | 7477 | UUUAAUGCUUCCCCAC | 417 |
| | | | AACdTdT | | | ACCdTdT | |
| 261 | D-2773 | 7478 | GACUGACUGAAGGCCU | 418 | 7479 | UGCAGGCCUUCAGUCA | 419 |
| | | | GCCdTdT | | | GUCdTdT | |
| 263 | D-2774 | 7480 | CUGACUGAAGGCCUGC | 420 | 7481 | UAUGCAGGCCUUCAGU | 421 |
| | | | AUCdTdT | | | CAGdTdT | |
| 264 | D-2775 | 7482 | UGACUGAAGGCCUGCA | 422 | 7483 | UCAUGCAGGCCUUCAG | 423 |
| | | | UGCdTdT | | | UCAdTdT | |
| 268 | D-2776 | 7484 | UGAAGGCCUGCAUGGA | 424 | 7485 | UAAUCCAUGCAGGCCU | 425 |
| | | | UUCdTdT | | | UCAdTdT | |
| 269 | D-2777 | 7486 | GAAGGCCUGCAUGGAU | 426 | 7487 | UGAAUCCAUGCAGGCC | 427 |
| | | | UCCdTdT | | | UUCdTdT | |
| 276 | D-2778 | 7488 | UGCAUGGAUUCCAUGU | 428 | 7489 | UGAACAUGGAAUCCAU | 429 |
| | | | UCCdTdT | | | GCAdTdT | |
| 278 | D-2779 | 7490 | CAUGGAUUCCAUGUUC | 430 | 7491 | UAUGAACAUGGAAUCC | 431 |
| | | | AUCdTdT | | | AUGdTdT | |
| 281 | D-2780 | 7492 | GGAUUCCAUGUUCAUG | 432 | 7493 | UCUCAUGAACAUGGAA | 433 |
| | | | AGCdTdT | | | UCCdTdT | |
| 284 | D-2781 | 7494 | UUCCAUGUUCAUGAGU | 434 | 7495 | UAAACUCAUGAACAUG | 435 |
| | | | UUCdTdT | | | GAAdTdT | |
| 290 | D-2782 | 7496 | GUUCAUGAGUUUGGAG | 436 | 7497 | UAUCUCCAAACUCAUG | 437 |
| | | | AUCdTdT | | | AACdTdT | |
| 291 | D-2783 | 7498 | UUCAUGAGUUUGGAGA | 438 | 7499 | UUAUCUCCAAACUCAU | 439 |
| | | | UACdTdT | | | GAAdTdT | |
| 295 | D-2784 | 7500 | UGAGUUUGGAGAUAAU | 440 | 7501 | UGUAUUAUCUCCAAAC | 441 |
| | | | ACCdTdT | | | UCAdTdT | |
| 296 | D-2785 | 7502 | GAGUUUGGAGAUAAUA | 442 | 7503 | UUGUAUUAUCUCCAAA | 443 |
| | | | CACdTdT | | | CUCdTdT | |
| 316 | D-2786 | 7504 | AGGCUGUACCAGUGCA | 444 | 7505 | UCCUGCACUGGUACAG | 445 |
| | | | GGCdTdT | | | CCUdTdT | |
| 317 | D-2787 | 7506 | GGCUGUACCAGUGCAG | 446 | 7507 | UACCUGCACUGGUACA | 447 |
| | | | GUCdTdT | | | GCCdTdT | |
| 329 | D-2788 | 7508 | GCAGGUCCUCACUUUA | 448 | 7509 | UAUUAAAGUGAGGACC | 449 |
| | | | AUCdTdT | | | UGCdTdT | |
| 330 | D-2789 | 7510 | CAGGUCCUCACUUUAA | 450 | 7511 | UGAUUAAAGUGAGGAC | 451 |
| | | | UCCdTdT | | | CUGdTdT | |
| 337 | D-2790 | 7512 | UCACUUUAAUCCUCUA | 452 | 7513 | UGAUAGAGGAUUAAAG | 453 |
| | | | UCCdTdT | | | UGAdTdT | |
| 350 | D-2791 | 7514 | CUAUCCAGAAAACACG | 454 | 7515 | UACCGUGUUUUCUGGA | 455 |
| | | | GUCdTdT | | | UAGdTdT | |
| 351 | D-2792 | 7516 | UAUCCAGAAAACACGG | 456 | 7517 | UCACCGUGUUUUCUGG | 457 |
| | | | UGCdTdT | | | AUAdTdT | |
| 352 | D-2793 | 7518 | AUCCAGAAAACACGGU | 458 | 7519 | UCCACCGUGUUUUCUG | 459 |
| | | | GGCdTdT | | | GAUdTdT | |
| 354 | D-2794 | 7520 | CCAGAAAACACGGUGG | 460 | 7521 | UGCCCACCGUGUUCUG | 461 |
| | | | GCCdTdT | | | UUUCUGGdTdT | |
| 357 | D-2795 | 7522 | GAAAACACGGUGGGCC | 462 | 7523 | UUUGGCCCACCGUGUU | 463 |
| | | | AACdTdT | | | UUCdTdT | |
| 358 | D-2796 | 7524 | AAAACACGGUGGGCCA | 464 | 7525 | UUUUGGCCCACCGUGU | 465 |
| | | | AACdTdT | | | UUUdTdT | |
| 364 | D-2797 | 7526 | CGGUGGGCCAAAGGAU | 466 | 7527 | UUCAUCCUUUGGCCCA | 467 |
| | | | GACdTdT | | | CCGdTdT | |
| 375 | D-2798 | 7528 | AGGAUGAAGAGAGGCA | 468 | 7529 | UCAUGCCUCUCUUCAU | 469 |
| | | | UGCdTdT | | | CCUdTdT | |
| 378 | D-2799 | 7530 | AUGAAGAGAGGCAUGU | 470 | 7531 | UCAACAUGCCUCUCUU | 471 |
| | | | UGCdTdT | | | CAUdTdT | |
| 383 | D-2800 | 7532 | GAGAGGCAUGUUGGAG | 472 | 7533 | UGUCUCCAACAUGCCU | 473 |
| | | | ACCdTdT | | | CUCdTdT | |
| 384 | D-2801 | 7534 | AGAGGCAUGUUGGAGA | 474 | 7535 | UAGUCUCCAACAUGCC | 475 |
| | | | CUCdTdT | | | UCUdTdT | |
| 390 | D-2802 | 7536 | AUGUUGGAGACUUGGG | 476 | 7537 | UUGCCCAAGUCUCCAA | 477 |
| | | | CACdTdT | | | CAUdTdT | |
| 392 | D-2803 | 7538 | GUUGGAGACUUGGGCA | 478 | 7539 | UAUUGCCCAAGUCUCC | 479 |
| | | | AUCdTdT | | | AACdTdT | |
| 395 | D-2804 | 7540 | GGAGACUUGGGCAAUG | 480 | 7541 | UCACAUUGCCCAAGUC | 481 |
| | | | UGCdTdT | | | UCCdTdT | |
| 404 | D-2805 | 7542 | GGCAAUGUGACUGCUG | 482 | 7543 | UGUCAGCAGUCACAUU | 483 |
| | | | ACCdTdT | | | GCCdTdT | |
| 406 | D-2806 | 7544 | CAAUGUGACUGCUGAC | 484 | 7545 | UUUGUCAGCAGUCACA | 485 |
| | | | AACdTdT | | | UUGdTdT | |
| 417 | D-2807 | 7546 | CUGACAAAGAUGGUGU | 486 | 7547 | UCCACACCAUCUUUGU | 487 |
| | | | GGCdTdT | | | CAGdTdT | |
| 418 | D-2808 | 7548 | UGACAAAGAUGGUGUG | 488 | 7549 | UGCCACACCAUCUUUG | 489 |
| | | | GCCdTdT | | | UCAdTdT | |
| 469 | D-2809 | 7550 | CUCAGGAGACCAUUGC | 490 | 7551 | UAUGCAAUGGUCUCCU | 491 |
| | | | AUCdTdT | | | GAGdTdT | |
| 470 | D-2810 | 7552 | UCAGGAGACCAUUGCA | 492 | 7553 | UGAUGCAAUGGUCUCC | 493 |
| | | | UCCdTdT | | | UGAdTdT | |
| 475 | D-2811 | 7554 | AGACCAUUGCAUCAUU | 494 | 7555 | UCCAAUGAUGCAAUGG | 495 |
| | | | GGCdTdT | | | UCUdTdT | |
| 476 | D-2812 | 7556 | GACCAUUGCAUCAUUG | 496 | 7557 | UGCCAAUGAUGCAAUG | 497 |
| | | | GCCdTdT | | | GUCdTdT | |
| 480 | D-2813 | 7558 | AUUGCAUCAUUGGCCG | 498 | 7559 | UUGCGGCCAAUGAUGC | 499 |
| | | | CACdTdT | | | AAUdTdT | |
| 487 | D-2814 | 7560 | CAUUGGCCGCACACUG | 500 | 7561 | UACCAGUGUGCGGCCA | 501 |
| | | | GUCdTdT | | | AUGdTdT | |
| 494 | D-2815 | 7562 | CGCACACUGGUGGUCC | 502 | 7563 | UAUGGACCACCAGUGU | 503 |
| | | | AUCdTdT | | | GCGdTdT | |
| 496 | D-2816 | 7564 | CACACUGGUGGUCCAU | 504 | 7565 | UUCAUGGACCACCAGU | 505 |
| | | | GACdTdT | | | GUGdTdT | |
| 497 | D-2817 | 7566 | ACACUGGUGGUCCAUG | 506 | 7567 | UUUCAUGGACCACCAG | 507 |
| | | | AACdTdT | | | UGUdTdT | |
| 501 | D-2818 | 7568 | UGGUGGUCCAUGAAAA | 508 | 7569 | UCUUUUUCAUGGACCA | 509 |
| | | | AGCdTdT | | | CCAdTdT | |
| 504 | D-2819 | 7570 | UGGUCCAUGAAAAAGC | 510 | 7571 | UCUGCUUUUUCAUGGA | 511 |
| | | | AGCdTdT | | | CCAdTdT | |
| 515 | D-2820 | 7572 | AAAGCAGAUGACUUGG | 512 | 7573 | UGCCCAAGUCAUCUGC | 513 |
| | | | GCCdTdT | | | UUUdTdT | |
| 518 | D-2821 | 7574 | GCAGAUGACUUGGGCA | 514 | 7575 | UUUUGCCCAAGUCAUC | 515 |
| | | | AACdTdT | | | UGCdTdT | |
| 522 | D-2822 | 7576 | AUGACUUGGGCAAAGG | 516 | 7577 | UCACCUUUGCCCAAGU | 517 |
| | | | UGCdTdT | | | CAUdTdT | |
| 523 | D-2823 | 7578 | UGACUUGGGCAAAGGU | 518 | 7579 | UCCACCUUUGCCCAAG | 519 |
| | | | GGCdTdT | | | UCAdTdT | |
| 524 | D-2824 | 7580 | GACUUGGGCAAAGGUG | 520 | 7581 | UUCCACCUUUGCCCAA | 521 |
| | | | GACdTdT | | | GUCdTdT | |
| 552 | D-2825 | 7582 | GUACAAAGACAGGAAA | 522 | 7583 | UCGUUUCCUGUCUUUG | 523 |
| | | | CGCdTdT | | | UACdTdT | |
| 554 | D-2826 | 7584 | ACAAAGACAGGAAACG | 524 | 7585 | UAGCGUUUCCUGUCUU | 525 |
| | | | CUCdTdT | | | UGUdTdT | |
| 555 | D-2827 | 7586 | CAAAGACAGGAAACGC | 526 | 7587 | UCAGCGUUUCCUGUCU | 527 |
| | | | UGCdTdT | | | UUGdTdT | |
| 562 | D-2828 | 7588 | AGGAAACGCUGGAAGU | 528 | 7589 | UCGACUUCCAGCGUUU | 529 |
| | | | CGCdTdT | | | CCUdTdT | |
| 576 | D-2829 | 7590 | GUCGUUUGGCUUGUGG | 530 | 7591 | UCACCACAAGCCAAAC | 531 |
| | | | UGCdTdT | | | GACdTdT | |
| 577 | D-2830 | 7592 | UCGUUUGGCUUGUGGU | 532 | 7593 | UACACCACAAGCCAAA | 533 |
| | | | GUCdTdT | | | CGAdTdT | |
| 578 | D-2831 | 7594 | CGUUUGGCUUGUGGUG | 534 | 7595 | UUACACCACAAGCCAA | 535 |
| | | | UACdTdT | | | ACGdTdT | |
| 579 | D-2832 | 7596 | GUUUGGCUUGUGGUGU | 536 | 7597 | UUUACACCACAAGCCA | 537 |
| | | | AACdTdT | | | AACdTdT | |
| 581 | D-2833 | 7598 | UUGGCUUGUGGUGUAA | 538 | 7599 | UAAUUACACCACAAGC | 539 |
| | | | UUCdTdT | | | CAAdTdT | |
| 583 | D-2834 | 7600 | GGCUUGUGGUGUAAUU | 540 | 7601 | UCCAAUUACACCACAA | 541 |
| | | | GGCdTdT | | | GCCdTdT | |
| 584 | D-2835 | 7602 | GCUUGUGGUGUAAUUG | 542 | 7603 | UCCCAAUUACACCACA | 543 |
| | | | GGCdTdT | | | AGCdTdT | |
| 585 | D-2836 | 7604 | CUUGUGGUGUAAUUGG | 544 | 7605 | UUCCCAAUUACACCAC | 545 |
| | | | GACdTdT | | | AAGdTdT | |
| 587 | D-2837 | 7606 | UGUGGUGUAAUUGGGA | 546 | 7607 | UGAUCCCAAUUACACC | 547 |
| | | | UCCdTdT | | | ACAdTdT | |
| 588 | D-2838 | 7608 | GUGGUGUAAUUGGGAU | 548 | 7609 | UCGAUCCCAAUUACAC | 549 |
| | | | CGCdTdT | | | CACdTdT | |
| 589 | D-2839 | 7610 | UGGUGUAAUUGGGAUC | 550 | 7611 | UGCGAUCCCAAUUACA | 551 |
| | | | GCCdTdT | | | CCAdTdT | |
| 593 | D-2840 | 7612 | GUAAUUGGGAUCGCCC | 552 | 7613 | UUUGGGCGAUCCCAAU | 553 |
| | | | AACdTdT | | | UACdTdT | |
| 594 | D-2841 | 7614 | UAAUUGGGAUCGCCCA | 554 | 7615 | UAUUGGGCGAUCCCAA | 555 |
| | | | AUCdTdT | | | UUAdTdT | |
| 595 | D-2842 | 7616 | AAUUGGGAUCGCCCAA | 556 | 7617 | UUAUUGGGCGAUCCCA | 557 |
| | | | UACdTdT | | | AUUdTdT | |
| 596 | D-2843 | 7618 | AUUGGGAUCGCCCAAU | 558 | 7619 | UUUAUUGGGCGAUCCC | 559 |
| | | | AACdTdT | | | AAUdTdT | |
| 597 | D-2844 | 7620 | UUGGGAUCGCCCAAUA | 560 | 7621 | UUUUAUUGGGCGAUCC | 561 |
| | | | AACdTdT | | | CAAdTdT | |
| 598 | D-2845 | 7622 | UGGGAUCGCCCAAUAA | 562 | 7623 | UGUUUAUUGGGCGAUC | 563 |
| | | | ACCdTdT | | | CCAdTdT | |
| 599 | D-2846 | 7624 | GGGAUCGCCCAAUAAA | 564 | 7625 | UUGUUUAUUGGGCGAU | 565 |
| | | | CACdTdT | | | CCCdTdT | |
| 602 | D-2847 | 7626 | AUCGCCCAAUAAACAU | 566 | 7627 | UGAAUGUUUAUUGGGC | 567 |
| | | | UCCdTdT | | | GAUdTdT | |
| 607 | D-2848 | 7628 | CCAAUAAACAUUCCCU | 568 | 7629 | UCAAGGGAAUGUUUAU | 569 |
| | | | UGCdTdT | | | UGGdTdT | |
| 608 | D-2849 | 7630 | CAAUAAACAUUCCCUU | 570 | 7631 | UCCAAGGGAAUGUUUA | 571 |
| | | | GGCdTdT | | | UUGdTdT | |
| 609 | D-2850 | 7632 | AAUAAACAUUCCCUUG | 572 | 7633 | UUCCAAGGGAAUGUUU | 573 |
| | | | GACdTdT | | | AUUdTdT | |
| 610 | D-2851 | 7634 | AUAAACAUUCCCUUGG | 574 | 7635 | UAUCCAAGGGAAUGUU | 575 |
| | | | AUCdTdT | | | UAUdTdT | |
| 611 | D-2852 | 7636 | UAAACAUUCCCUUGGA | 576 | 7637 | UCAUCCAAGGGAAUGU | 577 |
| | | | UGCdTdT | | | UUAdTdT | |
| 612 | D-2853 | 7638 | AAACAUUCCCUUGGAU | 578 | 7639 | UACAUCCAAGGGAAUG | 579 |
| | | | GUCdTdT | | | UUUdTdT | |
| 613 | D-2854 | 7640 | AACAUUCCCUUGGAUG | 580 | 7641 | UUACAUCCAAGGGAAU | 581 |
| | | | UACdTdT | | | GUUdTdT | |
| 616 | D-2855 | 7642 | AUUCCCUUGGAUGUAG | 582 | 7643 | UGACUACAUCCAAGGG | 583 |
| | | | UCCdTdT | | | AAUdTdT | |
| 621 | D-2856 | 7644 | CUUGGAUGUAGUCUGA | 584 | 7645 | UCCUCAGACUACAUCC | 585 |
| | | | GGCdTdT | | | AAGdTdT | |
| 633 | D-2857 | 7646 | CUGAGGCCCCUUAACU | 586 | 7647 | UUGAGUUAAGGGGCCU | 587 |
| | | | CACdTdT | | | CAGdTdT | |
| 635 | D-2858 | 7648 | GAGGCCCCUUAACUCA | 588 | 7649 | UGAUGAGUUAAGGGGC | 589 |
| | | | UCCdTdT | | | CUCdTdT | |
| 636 | D-2859 | 7650 | AGGCCCCUUAACUCAU | 590 | 7651 | UAGAUGAGUUAAGGGG | 591 |
| | | | CUCdTdT | | | CCUdTdT | |
| 639 | D-2860 | 7652 | CCCCUUAACUCAUCUG | 592 | 7653 | UAACAGAUGAGUUAAG | 593 |
| | | | UUCdTdT | | | GGGdTdT | |
| 640 | D-2861 | 7654 | CCCUUAACUCAUCUGU | 594 | 7655 | UUAACAGAUGAGUUAA | 595 |
| | | | UACdTdT | | | GGGdTdT | |
| 641 | D-2862 | 7656 | CCUUAACUCAUCUGUU | 596 | 7657 | UAUAACAGAUGAGUUA | 597 |
| | | | AUCdTdT | | | AGGdTdT | |
| 642 | D-2863 | 7658 | CUUAACUCAUCUGUUA | 598 | 7659 | UGAUAACAGAUGAGUU | 599 |
| | | | UCCdTdT | | | AAGdTdT | |
| 643 | 2864 | 7660 | UUAACUCAUCUGUUAU | 600 | 7661 | UGGAUAACAGAUGAGU | 601 |
| | | | CCCdTdT | | | UAAdTdT | |
| 644 | D-2865 | 7662 | UAACUCAUCUGUUAUC | 602 | 7663 | UAGGAUAACAGAUGAG | 603 |
| | | | CUCdTdT | | | UUAdTdT | |
| 645 | D-2866 | 7664 | AACUCAUCUGUUAUCC | 604 | 7665 | UCAGGAUAACAGAUGA | 605 |
| | | | UGCdTdT | | | GUUdTdT | |
| 654 | D-2867 | 7666 | GUUAUCCUGCUAGCUG | 606 | 7667 | UUACAGCUAGCAGGAU | 607 |
| | | | UACdTdT | | | AACdTdT | |
| 660 | D-2868 | 7668 | CUGCUAGCUGUAGAAA | 608 | 7669 | UCAUUUCUACAGCUAG | 609 |
| | | | UGCdTdT | | | CAGdTdT | |
| 661 | D-2869 | 7670 | UGCUAGCUGUAGAAAU | 610 | 7671 | UACAUUUCUACAGCUA | 611 |
| | | | GUCdTdT | | | GCAdTdT | |
| 666 | D-2870 | 7672 | GCUGUAGAAAUGUAUC | 612 | 7673 | UAGGAUACAUUUCUAC | 613 |
| | | | CUCdTdT | | | AGCdTdT | |
| 667 | D-2871 | 7674 | CUGUAGAAAUGUAUCC | 614 | 7675 | UCAGGAUACAUUUCUA | 615 |
| | | | UGCdTdT | | | CAGdTdT | |
| 668 | D-2872 | 7676 | UGUAGAAAUGUAUCCU | 616 | 7677 | UUCAGGAUACAUUUCU | 617 |
| | | | GACdTdT | | | ACAdTdT | |
| 669 | D-2873 | 7678 | GUAGAAAUGUAUCCUG | 618 | 7679 | UAUCAGGAUACAUUUC | 619 |
| | | | AUCdTdT | | | UACdTdT | |
| 673 | D-2874 | 7680 | AAAUGUAUCCUGAUAA | 620 | 7681 | UGUUUAUCAGGAUACA | 621 |
| | | | ACCdTdT | | | UUUdTdT | |
| 677 | D-2875 | 7682 | GUAUCCUGAUAAACAU | 622 | 7683 | UUAAUGUUUAUCAGGA | 623 |
| | | | UACdTdT | | | UACdTdT | |
| 692 | D-2876 | 7684 | UUAAACACUGUAAUCU | 624 | 7685 | UUAAGAUUACAGUGUU | 625 |
| | | | UACdTdT | | | UAAdTdT | |
| 698 | D-2877 | 7686 | ACUGUAAUCUUAAAAG | 626 | 7687 | UCACUUUUAAGAUUAC | 627 |
| | | | UGCdTdT | | | AGUdTdT | |
| 699 | D-2878 | 7688 | CUGUAAUCUUAAAAGU | 628 | 7689 | UACACUUUUAAGAUUA | 629 |
| | | | GUCdTdT | | | CAGdTdT | |
| 700 | D-2879 | 7690 | UGUAAUCUUAAAAGUG | 630 | 7691 | UUACACUUUUAAGAUU | 631 |
| | | | UACdTdT | | | ACAdTdT | |
| 701 | D-2880 | 7692 | GUAAUCUUAAAAGUGU | 632 | 7693 | UUUACACUUUUAAGAU | 633 |
| | | | AACdTdT | | | UACdTdT | |
| 706 | D-2881 | 7694 | CUUAAAAGUGUAAUUG | 634 | 7695 | UCACAAUUACACUUUU | 635 |
| | | | UGCdTdT | | | AAGdTdT | |
| 749 | D-2882 | 7696 | UACCUGUAGUGAGAAA | 636 | 7697 | UAGUUUCUCACUACAG | 637 |
| | | | CUCdTdT | | | GUAdTdT | |
| 770 | D-2883 | 7698 | UUAUGAUCACUUGGAA | 638 | 7699 | UUCUUCCAAGUGAUCA | 639 |
| | | | GACdTdT | | | UAAdTdT | |
| 772 | D-2884 | 7700 | AUGAUCACUUGGAAGA | 640 | 7701 | UAAUCUUCCAAGUGAU | 641 |
| | | | UUCdTdT | | | CAUdTdT | |
| 775 | D-2885 | 7702 | AUCACUUGGAAGAUUU | 642 | 7703 | UACAAAUCUUCCAAGU | 643 |
| | | | GUCdTdT | | | GAUdTdT | |
| 781 | D-2886 | 7704 | UGGAAGAUUUGUAUAG | 644 | 7705 | UAACUAUACAAAUCUU | 645 |
| | | | UUCdTdT | | | CCAdTdT | |
| 800 | D-2887 | 7706 | UAUAAAACUCAGUUAA | 646 | 7707 | UUUUUAACUGAGUUUU | 647 |
| | | | AACdTdT | | | AUAdTdT | |
| 804 | D-2888 | 7708 | AAACUCAGUUAAAAUG | 648 | 7709 | UGACAUUUUAACUGAG | 649 |
| | | | UCCdTdT | | | UUUdTdT | |
| 819 | D-2889 | 7710 | GUCUGUUUCAAUGACC | 650 | 7711 | UCAGGUCAUUGAAACA | 651 |
| | | | UGCdTdT | | | GACdTdT | |
| 829 | D-2890 | 7712 | AUGACCUGUAUUUUGC | 652 | 7713 | UUGGCAAAAUACAGGU | 653 |
| | | | CACdTdT | | | CAUdTdT | |
| 832 | D-2891 | 7714 | ACCUGUAUUUUGCCAG | 654 | 7715 | UGUCUGGCAAAAUACA | 655 |
| | | | ACCdTdT | | | GGUdTdT | |
| 833 | D-2892 | 7716 | CCUGUAUUUUGCCAGA | 656 | 7717 | UAGUCUGGCAAAAUAC | 657 |
| | | | CUCdTdT | | | AGGdTdT | |
| 851 | D-2893 | 7718 | UAAAUCACAGAUGGGU | 658 | 7719 | UAUACCCAUCUGUGAU | 659 |
| | | | AUCdTdT | | | UUAdTdT | |
| 854 | D-2894 | 7720 | AUCACAGAUGGGUAUU | 660 | 7721 | UUUAAUACCCAUCUGU | 661 |
| | | | AACdTdT | | | GAUdTdT | |
| 855 | D-2895 | 7722 | UCACAGAUGGGUAUUA | 662 | 7723 | UUUUAAUACCCAUCUG | 663 |
| | | | AACdTdT | | | UGAdTdT | |
| 857 | D-2896 | 7724 | ACAGAUGGGUAUUAAA | 664 | 7725 | UAGUUUAAUACCCAUC | 665 |
| | | | CUCdTdT | | | UGUdTdT | |
| 858 | D-2897 | 7726 | CAGAUGGGUAUUAAAC | 666 | 7727 | UAAGUUUAAUACCCAU | 667 |
| | | | UUCdTdT | | | CUGdTdT | |
| 859 | D-2898 | 7728 | AGAUGGGUAUUAAACU | 668 | 7729 | UCAAGUUUAAUACCCA | 669 |
| | | | UGCdTdT | | | UCUdTdT | |
| 861 | D-2899 | 7730 | AUGGGUAUUAAACUUG | 670 | 7731 | UGACAAGUUUAAUACC | 671 |
| | | | UCCdTdT | | | CAUdTdT | |
| 869 | D-2900 | 7732 | UAAACUUGUCAGAAUU | 672 | 7733 | UGAAAUUCUGACAAGU | 673 |
| | | | UCCdTdT | | | UUAdTdT | |
| 891 | D-2901 | 7734 | UCAUUCAAGCCUGUGA | 674 | 7735 | UAUUCACAGGCUUGAA | 675 |
| | | | AUCdTdT | | | UGAdTdT | |
| 892 | D-2902 | 7736 | CAUUCAAGCCUGUGAA | 676 | 7737 | UUAUUCACAGGCUUGA | 677 |
| | | | UACdTdT | | | AUGdTdT | |
| 906 | D-2903 | 7738 | AAUAAAAACCCUGUAU | 678 | 7739 | UCCAUACAGGGUUUUU | 679 |
| | | | GGCdTdT | | | AUUdTdT | |
| 907 | D-2904 | 7740 | AUAAAAACCCUGUAUG | 680 | 7741 | UGCCAUACAGGGUUUU | 681 |
| | | | GCCdTdT | | | UAUdTdT | |
| 912 | D-2905 | 7742 | AACCCUGUAUGGCACU | 682 | 7743 | UUAAGUGCCAUACAGG | 683 |
| | | | UACdTdT | | | GUUdTdT | |
| 913 | D-2906 | 7744 | ACCCUGUAUGGCACUU | 684 | 7745 | UAUAAGUGCCAUACAG | 685 |
| | | | AUCdTdT | | | GGUdTdT | |
| 934 | D-2907 | 7746 | GAGGCUAUUAAAAGAA | 686 | 7747 | UGAUUCUUUUAAUAGC | 687 |
| | | | UCCdTdT | | | CUCdTdT | |
| 944 | D-2908 | 7748 | AAAGAAUCCAAAUUCA | 688 | 7749 | UUUUGAAUUUGGAUUC | 689 |
| | | | AACdTdT | | | UUUdTdT | |
| 947 | D-2909 | 7750 | GAAUCCAAAUUCAAAC | 690 | 7751 | UUAGUUUGAAUUUGGA | 691 |
| | | | UACdTdT | | | UUCdTdT | |

The siRNA are then annealed and tested for SOD1 knockdown.

### Example 4. Pri and Pre-microRNAs Targeting SOD1

The passenger-guide strand duplexes of the SOD1 siRNA found to be efficacious from the experiments in Example 3 are engineered into expression vectors and transfected into cells of the central nervous system or neuronal cell lines. Even though overhang utilized in the siRNA knockdown study is a canonical dTdT for siRNA, the overhang in the synthetic pri- or pre-miR may comprise any dinucleotide overhang.

The cells used may be primary cells or derived from induced pluripotent stem cells (iPS cells).

SOD1 knockdown is then measured and deep sequencing performed to determine the exact passenger and guide strand processed from each pri- or pre-microRNA administered in the expression vector.

A guide to passenger strand ratio is calculated to determine the efficiency of knockdown, e.g., of RNA Induced Silencing Complex (RISC) processing.

The N-terminus is sequenced to determine the cleavage site and to determine the percent homogeneous cleavage of the target. It is expected that cleavage will be higher than 90 percent.

HeLa cells are co-transfected in a parallel study to analyze in vitro knockdown of SOD1. A luciferase construct is used as a control to determine off-target effects.

Deep sequencing is again performed.

### Example 5. Pri and Pre-microRNAs Targeting SOD1

According to the present invention, pri and pre-microRNAs were designed. These are given in Tables 6A, 6B, 7A and 7B. The sequences are described in the 5′ to 3′ direction and the regions of the stem-loop structure are broken out in the table in that order. In Tables 7A and 7B, the “miR” component of the name of the sequence does not necessarily correspond to the sequence numbering of miRNA genes (e.g., VOYmiR-101 is the name of the sequence and does not necessarily mean that miR-101 is part of the sequence).

TABLE 6A
Pre-miR sequences (5′-3′)

| Name and | | SEQ | | SEQ | | SEQ |
|---|---|---|---|---|---|---|
| Folded Energy (E) | Passenger | ID NO | Loop | ID NO | Guide | ID NO |
| VOYpre-001_D- | CAAUGUGACUGC | 692 | UGUGACCU | 5 | UUUGUCAGCAG | 693 |
| 2806-Starting construct | UGACAACCC | | GG | | UCACAUUGUU | |
| (18 native nucleotides and | | | | | | |
| position 19 is C; 3′ | | | | | | |
| terminal CC dinucleotide) | | | | | | |
| E = −33.8 | | | | | | |
| VOYpre-002_D- | CAAUGUGACUGC | 694 | UGUGACCU | 5 | UUUGUCAGCAG | 693 |
| 2806_p19MMU (position 19 | UGACAAUCC | | GG | | UCACAUUGUU | |
| U to form mismatch) | | | | | | |
| E = −34.2 | | | | | | |
| VOYpre-003_D- | CAAUGUGACUGC | 695 | UGUGACCU | 5 | UUUGUCAGCAG | 693 |
| 2806-p19GUpair (position | UGACAAGCC | | GG | | UCACAUUGUU | |
| 19 is G to form GU pair) | | | | | | |
| E = −38.1 | | | | | | |
| VOYpre-004_D- | CAAUGUGACUGC | 696 | UGUGACCU | 5 | UUUGUCAGCAG | 693 |
| 2806_p19AUpair (position | UGACAAACC | | GG | | UCACAUUGUU | |
| 19 is A to form AU pair) | | | | | | |
| E = −38.1 | | | | | | |
| VOYpre-005_D-2806_CMM | CAAUGUGACAGC | 697 | UGUGACCU | 5 | UUUGUCAGCAG | 693 |
| (central mismatch) | UGACAAACC | | GG | | UCACAUUGUU | |
| E = −33.0 | | | | | | |
| VOYpre-006_D-2806_p19DEL | CAAUGUGACUGC | 698 | UGUGACCU | 5 | UUUGUCAGCAG | 693 |
| (position 19 deleted) | UGACAACC | | GG | | UCACAUUGUU | |
| E = −34.0 | | | | | | |
| VOYpre-007_D- | CAAUGUGACUGC | 699 | UGUGACCU | 5 | UUUGUCAGCAG | 693 |
| 2806_p19ADD (nucleotide | UGACAAUCCC | | GG | | UCACAUUGUU | |
| added at position 19; | | | | | | |
| addition is U; keep C and | | | | | | |
| terminal CC dinucleotide) | | | | | | |
| E = −32.8 | | | | | | |
| VOYpre-008_D-2806_Uloop | CAAUGUGACUGC | 692 | UGUGAUUU | 6 | UUUGUCAGCAG | 693 |
| (increase U content of | UGACAACCC | | GG | | UCACAUUGUU | |
| loop) | | | | | | |
| E = −33.8 | | | | | | |
| VOYpre-009_D- | CAAUGUGACUGC | 692 | UAUAAUUU | 7 | UUUGUCAGCAG | 693 |
| 2806_AUloop (increase AU | UGACAACCC | | GG | | UCACAUUGUU | |
| content of loop) | | | | | | |
| E = −33.8 | | | | | | |
| VOYpre-010_D-2806_mir- | CAAUGUGACUGC | 700 | CCUGACCC | 8 | UUUGUCAGCAG | 693 |
| 22-loop (swap in loop from | UGACAACAC | | AGU | | UCACAUUGUU | |
| miR-22) | | | | | | |
| E = −30.0 | | | | | | |

TABLE 6B
Pre-miR sequences (5′-3′)

| Name and | | SEQ | | SEQ | | SEQ |
|---|---|---|---|---|---|---|
| Folded Energy (E) | Guide | ID NO | Loop | ID NO | Passenger | ID NO |
| YOYpre-011_D- | UUUGUCAGC | 701 | UGUGACCU | 5 | CAAUGUGACUGCU | 702 |
| 2806_passenger-guide strand | AGUCACAUU | | GG | | GACAAAUC | |
| swap with terminal 3′ C on | GUC | | | | | |
| passenger strand | | | | | | |
| E = −36.1 | | | | | | |
| YOYpre-012_D- | UUUGUCAGC | 701 | UGUGACCU | 5 | CAAUGUGACUGCU | 703 |
| 2806_passenger-guide strand | AGUCACAUU | | GG | | GACAAUUC | |
| swap with terminal 3′ C on | GUC | | | | | |
| passenger strand | | | | | | |
| E = −35.4 | | | | | | |
| YOYpre-013_D- | UUUGUCAGC | 704 | CCUGACCC | 8 | CAAUGUGACUGCU | 702 |
| 2806_passenger-guide strand | AGUCACAUU | | AGU | | GACAAAUC | |
| swap with terminal 3′ C on | GAC | | | | | |
| passenger strand | | | | | | |
| E = −34.7 | | | | | | |

TABLE 7A
Pri-miR sequences (5′-3′)
Pri-miR construct components 5′ to 3′

| | 5′ Flank- | Passenger | Loop | Guide | | 5′ Flanking to |
|---|---|---|---|---|---|---|
| Name and Folded | ing SEQ | SEQ ID | SEQ ID | SEQ ID | 3′ Flanking | 3′ Flanking |
| Energy (E) | ID NO | NO | NO | NO | SEQ ID NO | SEQ ID NO |
| VOYmiR-101_pre-001 | 1 | 692 | 5 | 693 | 10 | 747 |
| hsa-mir-155; D-2806 | | | | | | |
| E = −63.7 | | | | | | |
| VOYmiR-102_pre-001 | 2 | 692 | 5 | 693 | 11 | 748 |
| Engineered; D-2806; | | | | | | |
| let-7b stem | | | | | | |
| E = −106.0 | | | | | | |
| VOYmiR-103_pre-002 | 2 | 694 | 5 | 693 | 11 | 749 |
| Engineered; D- | | | | | | |
| 2806_p19MMU; let-7b | | | | | | |
| stem | | | | | | |
| E = −106.4 | | | | | | |
| VOYmiR-104_pre-003 | 2 | 695 | 5 | 693 | 11 | 750 |
| Engineered; D- | | | | | | |
| 2806_p19GUpair; let- | | | | | | |
| 7b stem | | | | | | |
| E = −110.3 | | | | | | |
| VOYmiR-105_pre-004 | 2 | 696 | 5 | 693 | 11 | 751 |
| Engineered; D- | | | | | | |
| 2806_p19AUpair; let- | | | | | | |
| 7b stem | | | | | | |
| E = −110.3 | | | | | | |
| VOYmiR-106_pre-005 | 2 | 697 | 5 | 693 | 11 | 752 |
| Engineered; D- | | | | | | |
| 2806_CMM; let-7b | | | | | | |
| stem | | | | | | |
| E = −105.2 | | | | | | |
| VOYmiR-107_pre-006 | 2 | 698 | 5 | 693 | 11 | 753 |
| Engineered; D- | | | | | | |
| 2806_p19DEL; let-7b | | | | | | |
| stem | | | | | | |
| E = −106.2 | | | | | | |
| VOYmiR-108_pre-007 | 2 | 705 | 5 | 693 | 11 | 754 |
| Engineered; D- | | | | | | |
| 2806_p19ADD; let-7b | | | | | | |
| stem | | | | | | |
| E = −105.0 | | | | | | |
| VOYmiR-109_pre-008 | 2 | 692 | 6 | 693 | 11 | 755 |
| Engineered; D- | | | | | | |
| 2806_Uloop; let-7b | | | | | | |
| stem | | | | | | |
| E = −106.0 | | | | | | |
| VOYmiR-110_pre-009 | 2 | 692 | 7 | 693 | 11 | 756 |
| Engineered; D- | | | | | | |
| 2806_AUloop; let-7b | | | | | | |
| stem | | | | | | |
| E = −106.0 | | | | | | |
| VOYmiR-111_pre-010 | 2 | 700 | 8 | 693 | 11 | 757 |
| Engineered; D- | | | | | | |
| 2806_mir-22-loop; let- | | | | | | |
| 7b stem | | | | | | |
| E = −102.2 | | | | | | |
| VOYmiR-112_pre-001 | 2 | 692 | 5 | 693 | 12 | 758 |
| Engineered; PD; D- | | | | | | |
| 2806; let-7b basal-stem | | | | | | |
| instability | | | | | | |
| E = −102.3 | | | | | | |
| VOYmiR-113_pre-002 | 2 | 694 | 5 | 693 | 12 | 759 |
| Engineered; D- | | | | | | |
| 2806_p19MMU; let-7b | | | | | | |
| basal-stem instability | | | | | | |
| E = −102.7 | | | | | | |
| VOYmiR-114_pre-005 | 2 | 697 | 5 | 693 | 12 | 760 |
| Engineered; D- | | | | | | |
| 2806_CMM; let-7b | | | | | | |
| basal-stem instability | | | | | | |
| E = −101.5 | | | | | | |
| VOYmiR-115_pre-010 | 2 | 700 | 8 | 693 | 12 | 761 |
| Engineered; D- | | | | | | |
| 2806_mir-22-loop; let- | | | | | | |
| 7b basal-stem | | | | | | |
| instability | | | | | | |
| E = −98.5 | | | | | | |
| VOYmiR-116_pre-003 | 2 | 695 | 5 | 693 | 12 | 762 |
| Engineered; D- | | | | | | |
| 2806_p19GUpair; let- | | | | | | |
| 7b basal-stem | | | | | | |
| instability | | | | | | |
| E = −110.1 | | | | | | |
| VOYmiR-117_pre-001 | 2 | 706 | 5 | 707 | 11 | 763 |
| Engineered; D-2757; | | | | | | |
| let-7b stem | | | | | | |
| E = −106.9 | | | | | | |
| VOYmiR-118_pre-001 | 2 | 708 | 5 | 709 | 11 | 764 |
| Engineered; D-2823; | | | | | | |
| let-7b stem | | | | | | |
| E = −108.7 | | | | | | |
| VOYmiR-119_pre-001 | 2 | 710 | 5 | 711 | 11 | 765 |
| Engineered; D-2866; | | | | | | |
| let-7b stem | | | | | | |
| VOYmiR-127 | 3 | 692 | 9 | 693 | 13 | 766 |
| VOYmiR-102.860 | 2 | 712 | 5 | 713 | 11 | 767 |
| VOYmiR102.861 | 2 | 714 | 5 | 715 | 11 | 768 |
| VOYmiR-102.866 | 2 | 716 | 5 | 711 | 11 | 769 |
| VOYmiR-102.870 | 2 | 717 | 5 | 718 | 11 | 770 |
| VOYmiR-102.823 | 2 | 719 | 5 | 709 | 11 | 771 |
| VOYmiR-104.860 | 2 | 720 | 5 | 713 | 11 | 772 |
| VOYmiR-104.861 | 2 | 721 | 5 | 715 | 11 | 773 |
| VOYmiR-104.866 | 2 | 722 | 5 | 711 | 11 | 774 |
| VOYmiR-104.870 | 2 | 723 | 5 | 718 | 11 | 775 |
| VOYmiR-104.823 | 2 | 724 | 5 | 709 | 11 | 776 |
| VOYmiR-109.860 | 2 | 712 | 6 | 713 | 11 | 777 |
| VOYmiR-104.861 | 2 | 714 | 6 | 715 | 11 | 778 |
| VOYmiR-104.866 | 2 | 716 | 6 | 711 | 11 | 779 |
| VOYmiR-109.870 | 2 | 717 | 6 | 718 | 11 | 780 |
| VOYmiR-109.823 | 2 | 719 | 6 | 709 | 11 | 781 |
| VOYmiR-114.860 | 2 | 725 | 5 | 713 | 12 | 782 |
| VOYmiR-114.861 | 2 | 726 | 5 | 715 | 12 | 783 |
| VOYmiR-114.866 | 2 | 727 | 5 | 711 | 12 | 784 |
| VOYmiR-114.870 | 2 | 728 | 5 | 718 | 12 | 785 |
| VOYmiR-114.823 | 2 | 729 | 5 | 709 | 12 | 786 |
| VOYmiR-116.860 | 2 | 720 | 5 | 713 | 12 | 787 |
| VOYmiR-116.861 | 2 | 721 | 5 | 715 | 12 | 788 |
| VOYmiR-116.866 | 2 | 730 | 5 | 711 | 12 | 789 |
| VOYmiR-116.870 | 2 | 723 | 5 | 718 | 12 | 790 |
| VOYmiR-116.823 | 2 | 724 | 5 | 709 | 12 | 791 |
| VOYmiR-127.860 | 3 | 731 | 9 | 713 | 13 | 792 |
| VOYmiR-127.861 | 3 | 714 | 9 | 715 | 13 | 793 |
| VOYmiR-127.866 | 3 | 716 | 9 | 711 | 13 | 794 |
| VOYmiR-127.870 | 3 | 717 | 9 | 718 | 13 | 795 |
| VOYmiR-127.823 | 3 | 732 | 9 | 709 | 13 | 796 |

TABLE 7B
Pri-miR sequences (5′-3′)

| | | | | Passenger | | 5′ Flanking to |
|---|---|---|---|---|---|---|
| | 5′ Flanking | Guide SEQ | Loop SEQ | SEQ | 3′ Flanking | 3′ Flanking |
| Name | SEQ ID NO | ID NO | ID NO | ID NO | SEQ ID NO | SEQ ID NO |
| VOYmiR-120 | 4 | 733 | 5 | 734 | 810 | 797 |

### Example 6. Pri and Pre-microRNAs Targeting SOD1; In Vivo Study

In vivo studies are performed to test the efficacy of the pri- or pre-microRNA constructs of Example 5.

Table 8 outlines the experimental design variables to be explored.

The design of the modulatory nucleic acids (pri or pre-microRNA) includes a loop region derived from miR30, a stem region is derived from let7 and various combinations of passenger strands that vary in bulge, mismatch, and asymmetry regions.

TABLE 8
Experimental Design

| Variable | Options |
|---|---|
| AAV Serotype | AAVrh10, AAV9 |
| Species | NHP (non human primate), pig, |
| | sheep, rodent |
| Route of delivery | IT-lumbar, -thoracic, -cervical; CM |
| | Single site, multi-site |
| Vector concentration | 1 × 10¹³ vg/mL |
| Rate of infusion | Bolus (0.3-1 mL/min), 1 mL/hr |
| Duration of infusion | 1-3 min, 1 hour, 10 hours |
| Total dose | 3 × 10¹³ vg (vector genomes) |
| Position of animal | Prone, upright |
| Catheter | Implanted, acute/adjustable |
| Labelling of vector | No label, MRI-Gadolinium; PET-¹²⁴I |
| | or-zirconium |

### Example 7. Pri-miRNA Constructs in AAV-miRNA Vectors

The passenger-guide strand duplexes of the SOD1 siRNA listed in Table 7 are engineered into AAV-miRNA expression vectors. The construct from ITR to ITR, recited 5′ to 3′, comprises a mutant ITR, a promoter (either a CMV, a U6 or the CB6 promoter (which includes a CMVie enhancer, a CBA promoter and an SV40 intron), the pri-miRNA construct from Table 7, a rabbit globin polyA and wildtype ITR. In vitro and in vivo studies are performed to test the efficacy of the AAV-miRNA expression vectors.

### Example 8. Activity of Pri-miRNA Constructs in HeLa Cells

Seven of the pri-miRNA constructs described in Example 7 (VOYmiR-103, VOYmiR-105, VOYmiR-108, VOYmiR-114, VOYmiR-119, VOYmiR-120, and VOYmiR-127) and a control of double stranded mCherry were transfected in HeLa to test the activity of the constructs.

### A. Passenger and Guide Strand Activity

The seven pri-miRNA constructs and a control of double stranded mCherry were transfected into HeLa cells. After 48 hours the endogenous mRNA expression was evaluated. All seven of the pri-miRNA constructs showed high activity of the guide strand with 75-80% knock-down and low to no activity of the passenger strand. Guide strands of miRNA candidate vectors showed high activity, yielding 75-80% knockdown of SOD1, while passenger strands demonstrated little to no activity.

B. Activity of miRNA on SOD1

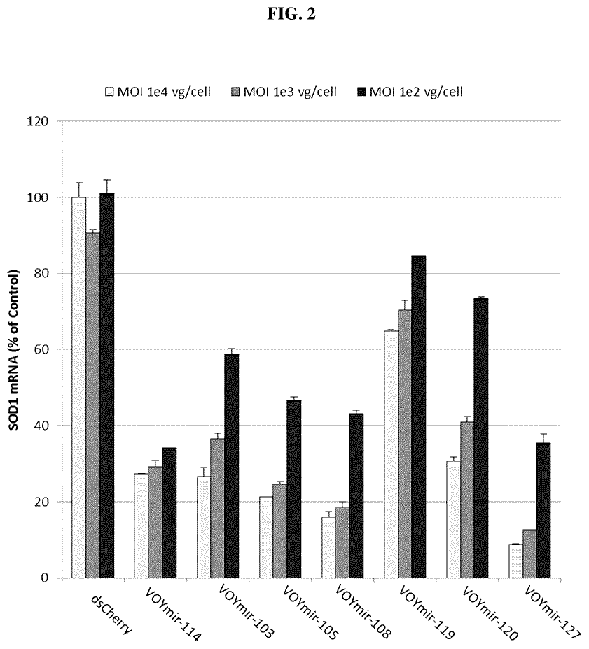

The seven pri-miRNA constructs and a control of double stranded mCherry (dsmCherry) were transfected into HeLa cells at a MOI of 1e4 vg/cell, 1e3 vg/cell, or 1e2 vg/cell. After 72 hours the endogenous mRNA expression was evaluated. All seven of the pri-miRNA constructs showed efficient knock-down at 1e3 vg/cell. Most of the pri-miRNA constructs showed high activity (75-80% knock-down) as shown in FIG. 2.

### Example 9. Activity of Pri-miRNA Constructs

Thirty of the pri-miRNA constructs described in Example 7 (VOYmiR-102.860, VOYmiR-102.861, VOYmiR-102.866, VOYmiR-102.870, VOYmiR-102.823, VOYmiR-104.860, VOYmiR-104.861, VOYmiR-104.866, VOYmiR-104.870, VOYmiR-104.823, VOYmiR-109.860, VOYmiR-109.861, VOYmiR-109.866, VOYmiR-109.870, VOYmiR-109.823, VOYmiR-114.860, VOYmiR-114.861, VOYmiR-114.866, VOYmiR-114.870, VOYmiR-114.823, VOYmiR-116.860, VOYmiR-116.861, VOYmiR-116.866, VOYmiR-116.870, VOYmiR-116.823, VOYmiR-127.860, VOYmiR-127.861, VOYmiR-127.866, VOYmiR-127.870, VOYmiR-127.823) and a control of VOYmiR-114 and double stranded mCherry were transfected in cells to test the activity of the constructs.

### A. Passenger and Guide Strand Activity in HEK293

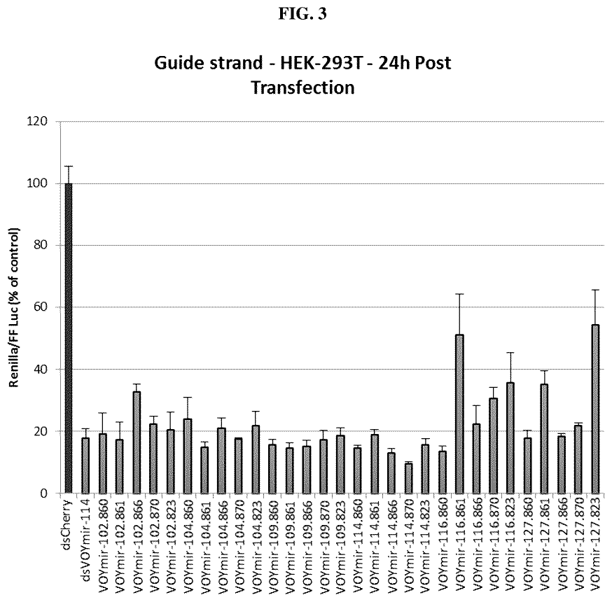

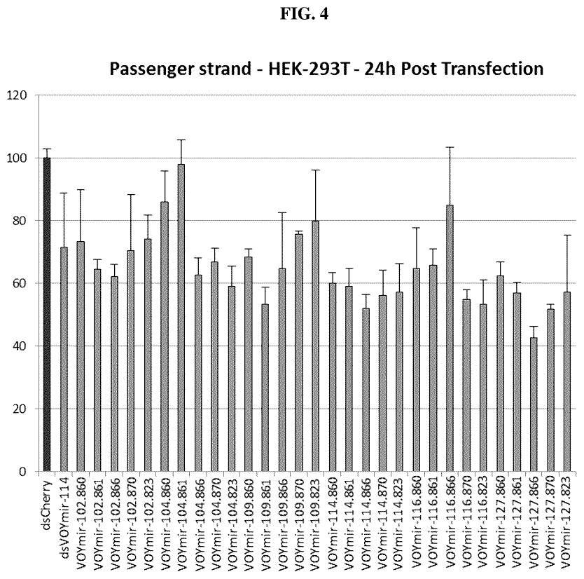

The thirty pri-miRNA constructs and two controls were transfected into HEK293T cells. After 24 hours the endogenous mRNA expression was evaluated. Most of the pri-mRNA constructs showed high activity of the guide strand (FIG. 3) and low to no activity of the passenger strand (FIG. 4).

### B. Passenger and Guide Strand Activity in HeLa

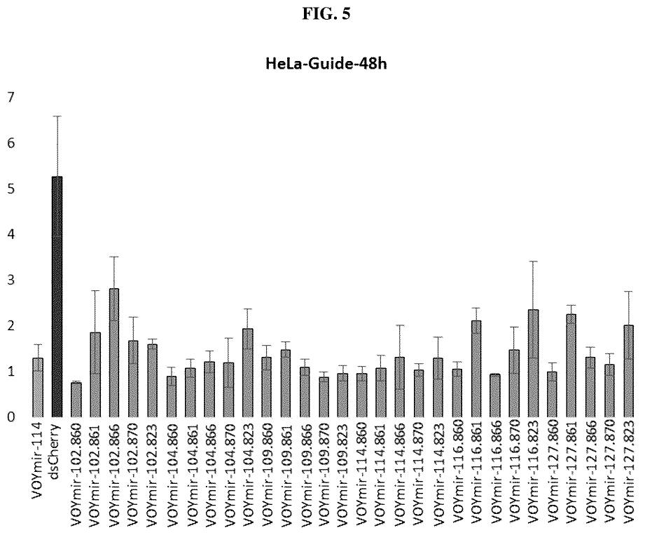

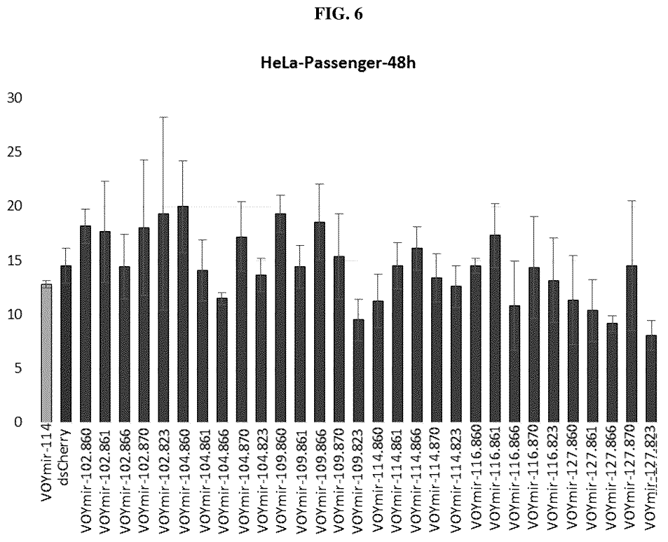

The thirty pri-miRNA constructs and two controls were transfected into HeLa cells. After 48 hours the endogenous mRNA expression was evaluated. Most of the pri-mRNA constructs showed high activity of the guide strand (FIG. 5) and low to no activity of the passenger strand (FIG. 6).

### C. HeLa and HEK293 Correlation

The knock-down of the thirty pri-miRNA were similar between the HeLa and HEK293 cells. The thirty pri-miRNA constructs showed knock-down for the guide strand of the constructs (See FIG. 3 and FIG. 5). Most of the guide strands of the pri-miRNA constructs showed 70-90% knock-down.

### D. Capsid Selection

The top pri-miRNA constructs from the HeLa and HEK293 are packaged in AAVs and will undergo HeLa infection. To determine the best AAV to package for the constructs, mCherry packaged in either AAV2 or AAV-DJ8 was infected into HeLa cells at a MOI of 10 vg/cell, 1e2 vg/cell, 1e3 vg/cell, 1e4 vg/cell or 1e5 vg/cell and the expression was evaluated at 40 hours. AAV2 was selected as the capsid to package the top pri-miR constructs.

### E. AAV2 Production

The top pri-miRNA constructs from the HeLa and HEK293 are packaged in AAV2 (1.6 kb) and a control of double stranded mCherry (dsmCherry) was also packaged. The packaged constructs underwent Idoixanol purification prior to analysis. The AAV titer is shown in Table 9.

TABLE 9
AAV Titer

| Construct | AAV Titer (genomes per ul) |
|---|---|
| VOYmir-102.860 | 5.5E+08 |
| VOYmir-102.861 | 1.0E+09 |
| VOYmir-102.823 | 9.1E+08 |
| VOYmir-104.861 | 1.2E+09 |
| VOYmir-104.866 | 8.0E+08 |
| VOYmir-104.823 | 5.7E+08 |
| VOYmir-109.860 | 3.1E+08 |
| VOYmir-109.861 | 8.9E+08 |
| VOYmir-109.866 | 6.0E+08 |
| VOYmir-109.823 | 6.0E+08 |
| VOYmir-114.860 | 4.7E+08 |
| VOYmir-114.861 | 3.7E+08 |
| VOYmir-114.866 | 1.0E+09 |
| VOYmir-144.823 | 1.7E+09 |
| VOYmir-116.860 | 1.0E+09 |
| VOYmir-116.866 | 9.1E+08 |
| VOYmir-127.860 | 1.2E+09 |
| VOYmir-127.866 | 9.0E+08 |
| dsmCherry | 1.2E+09 |

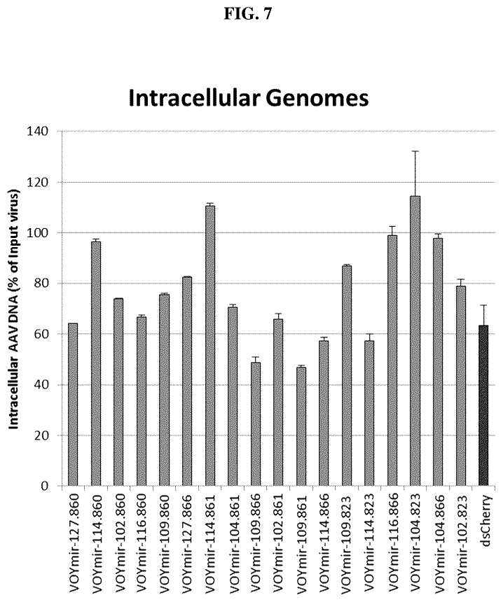

The effect of transduction on SOD1 knock-down in HeLa cells is shown in FIG. 7. In addition, in HeLa cells, a larger MOI (1.0E+04 compared to 1.0E+05) did not show increased knock-down for every construct.

### F. Activity of Constructs in Human Motor Neuron Progenitors (HMNPs)

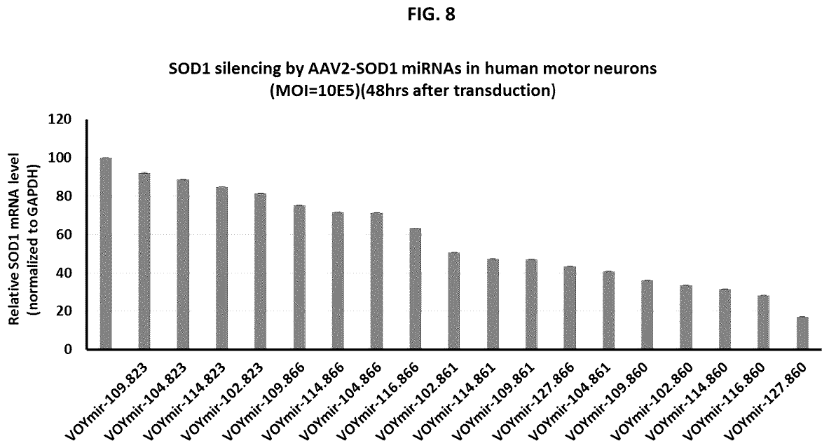

The top 18 pri-miRNA constructs as described in Example 9E and a control of mCherry were infected into human motor neuron progenitor (HMNP) cells at a MOI of 10E5. After 48 hours the endogenous mRNA expression was evaluated. About half of the constructs gave greater than 50% silencing of SOD1 in HMNPs and 4 of those gave greater than 70% silencing (FIG. 8).

### G. Construct Selection for In Vivo Studies

The top twelve pri-miRNA packaged constructs are selected which had a major effect on the target sequence and a minor effect on the cassette. These constructs packaged in AAV-rh10 capsids are formulated for injection and administered in mammals to study the in vivo effects of the constructs.

### H. Activity in Various Cell Lines

The activity of the pri-miRNA packaged constructs was tested in HeLa, SH-SY5Y, U87MG and primary human astrocyte cells. The activity in HeLa cells ranged from 1 to 5 pM. The activity in SH-SY5Y cells ranged from 13 to 17 pM. The activity in U87MG cells was about 1 pM. The activity in primary human astrocyte cells ranged from 49 to 123 pM.

### Example 10. In Vitro Study of Pri-miRNAs

The 18 pri-miRNAs and mCherry control described in Example 9D packaged in AAV2 were used for this study. For this study, HEK293T cells (Fisher Scientific, Cat. #HCL4517) in culture medium (500 ml of DMEM/F-12 GLUTAMAX™ supplement (Life Technologies, Cat #. 10565-018), 50 ml FBS (Life Technologies, Cat #. 16000-044, lot: 1347556), 5 ml MEM Non-essential amino acids solution (100×) (Cat. #11140-050) and 5 ml HEPES (1M) (Life Technologies, Cat #. 15630-080)), U251MG cells (P18) (Sigma, Cat #. 09063001-1VL) in culture medium (500 ml of DMEM/F-12 GLUTAMAX™ supplement (Life Technologies, Cat #. 10565-018), 50 ml FBS (Life Technologies, Cat #. 16000-044, lot: 1347556), 5 ml MEM Non-essential amino acids solution (100×) (Cat. #11140-050) and 5 ml HEPES (1M) (Life Technologies, Cat #. 15630-080)) or normal human astrocyte (HA) (Lonza, Cat #CC-2565) in culture medium (ABM Basal Medium 500 ml (Lonza, Cat #. CC-3186) supplemented with AGM SingleQuot Kit Suppl. & Growth Factors (Lonza, Cat #. CC-4123)) were used to test the constructs. HEK293T cells (5×10E4 cells/well in 96 well plate), U251MG cells (2×10E4 cells/well in 96 well plate) and HA cells (2×10E4 cells/well in 96 well plate) were seeded and the MOI used for infection of cells was 1.0E+05. After 48 hours the cells were analyzed and the results are shown in Table 10.

TABLE 10
Relative SOD1 mRNA level

| | Relative SOD1 mRNA Level (%) | | |
|---|---|---|---|
| | (Normalized to GAPDH) | | |
| Construct | HEK293T | U251MG | HA |
| VOYmiR-102.823 | 19.5 | 49.6 | 87.3 |
| VOYmiR-102.860 | 1.7 | 5.3 | 19.2 |
| VOYmiR-102.861 | 1.1 | 13.9 | 42.6 |
| VOYmiR-104.823 | 49.9 | 69.6 | 102.7 |
| VOYmiR-104.861 | 1.0 | 10.7 | 36.3 |
| VOYmiR-104.866 | 12.3 | 54.6 | 85.5 |
| VOYmiR-109.823 | 23.0 | 46.1 | 84.6 |
| VOYmiR-109.860 | 1.9 | 8.3 | 35.6 |
| VOYmiR-109.861 | 1.9 | 22.7 | 57.3 |
| VOYmiR-109.866 | 4.1 | 38.5 | 67.9 |
| VOYmiR-114.823 | 19.3 | 44.7 | 82.3 |
| VOYmiR-114.860 | 1.4 | 4.7 | 17.6 |
| VOYmiR-114.861 | 1.1 | 9.7 | 48.1 |
| VOYmiR-114.866 | 4.0 | 38.7 | 78.2 |
| VOYmiR-116.860 | 1.1 | 4.8 | 15.8 |
| VOYmiR-116.866 | 5.5 | 40.2 | 73.7 |
| VOYmiR-127.860 | 1.0 | 2.1 | 7.4 |
| VOYmiR-127.866 | 1.0 | 15.4 | 43.8 |
| mCherry | 100.0 | 100.2 | 100.1 |

Greater than 80% knock-down was seen in the HEK293T cells for most constructs. More than half of the constructs showed greater than 80% knock-down in the U251MG cells and in the HA cells.

### Example 11. Dose Dependent SOD1 Lowering

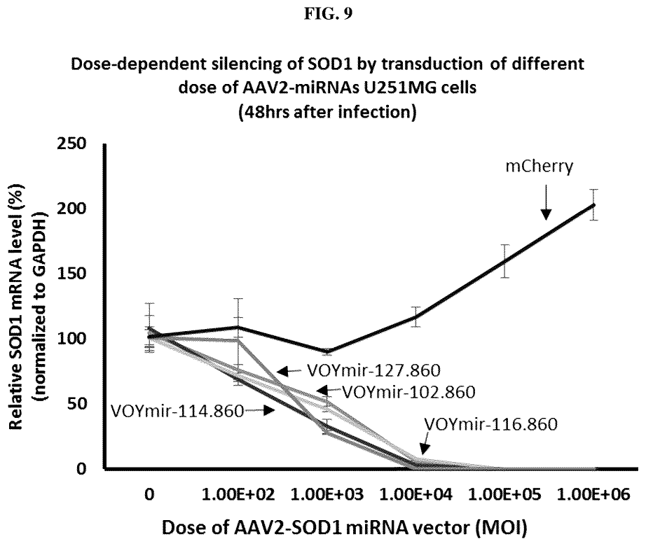

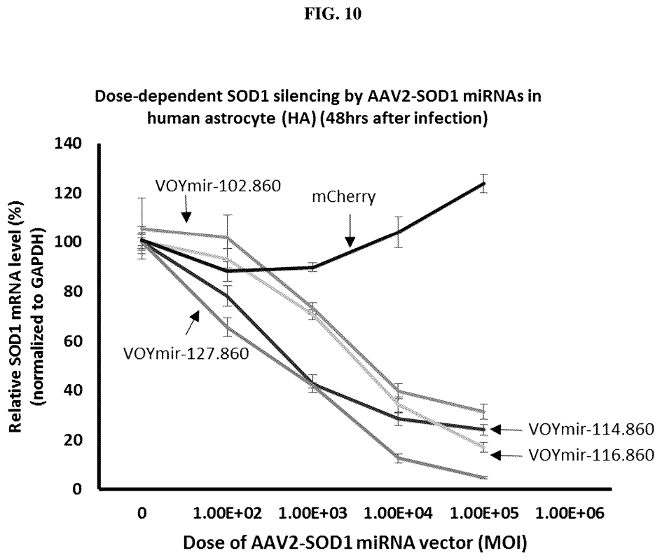

Four of the top 18 pri-miRNA constructs as described in Example 9E and a control of mCherry were transfected into a human astrocyte cell line (U251MG) or a primary human astrocyte (HA) at an MOI of 1.0E+02, 1.0E+03, 1.0E+04, 1.0E+05 or 1.0E+06. After 48 hours the endogenous mRNA expression and the dose-dependent silencing was evaluated and are shown in FIG. 9 (U251MG) and FIG. 10 (HA). For all constructs, the increase in dose also correlated to an increase in the amount of SOD1 mRNA that was knocked-down.

### Example 12. Time Course of SOD1 Knock-Down

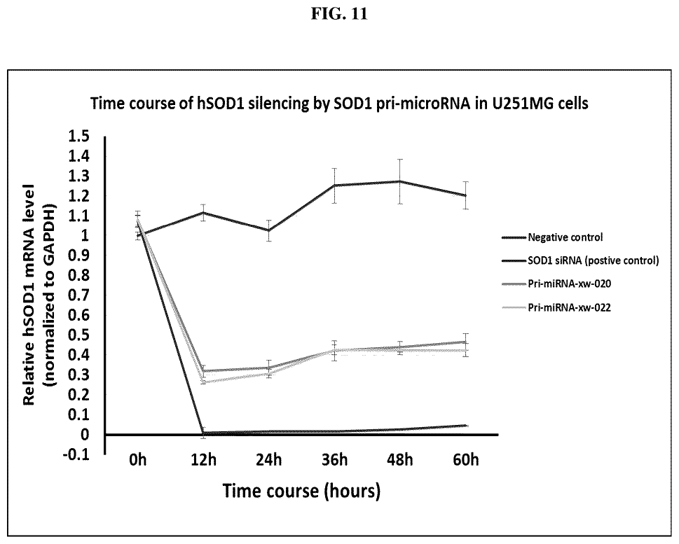

Two pri-miRNA constructs (VOYmiR-120 and VOYmiR-122), a negative control and a positive control of SOD1 siRNA were transfected into a human astrocyte cell line (U251MG). The relative SOD1 mRNA was determined for 60 hours as shown in FIG. 11. 70-75% knock-down of hSOD1 was seen for both pri-miR constructs after Nucleofector transfection, with the greatest knock-down seen in the 12-24 hour window.

### Example 13. SOD1 Knock-Down and Stand Percentages

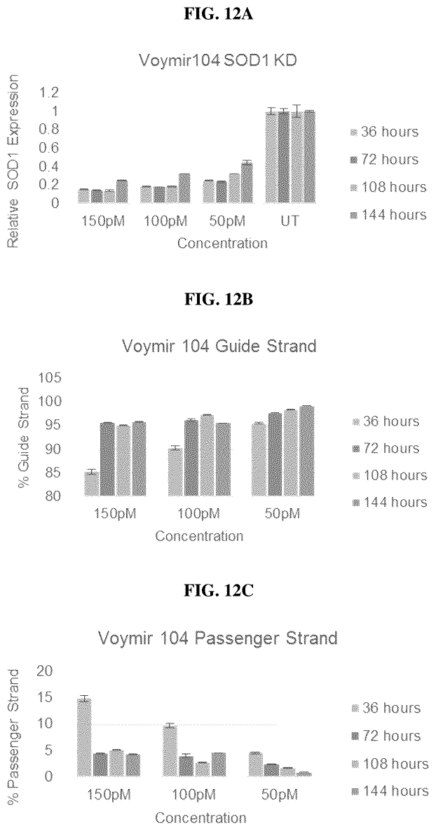

VOYmiR-104 was transfected into HeLa cells at concentrations of 50 pM, 100 pM and 150 pM and compared to untreated (UT) cells. The relative SOD1 mRNA, the percent of the guide strand and the percent of the passenger strand was determined at 36, 72, 108 and 144 hours as shown in FIGS. 12A-12C. The highest concentration (150 pM) showed the greatest reduction in expression, but all three doses showed a significant reduction in the expression of SOD1.

### Example 14. Pri-miRNAs Targeting SOD1

Pri-miRNAs were designed for Dog SOD1 and the constructs are given in Table 11. Dog SOD1 is 100% conserved with human in the region targeted in the present invention. The sequences are described in the 5′ to 3′ direction and the regions of the stem-loop structure are broken out in the table in that order. In Table 11, the “miR” component of the name of the sequence does not necessarily correspond to the sequence numbering of miRNA genes (e.g., dVOYmiR-102 is the name of the sequence and does not necessarily mean that miR-102 is part of the sequence).

TABLE 11
Dog Pri-miR sequences (5′-3′)
Pri-miR construct components 5′ to 3′

| | | | | | | 5′ Flanking to |
|---|---|---|---|---|---|---|
| | 5′ Flanking | Passenger SEQ | Loop SEQ | Guide SEQ | 3′ Flanking | 3′ Flanking |
| Name | SEQ ID NO | ID NO | ID NO | ID NO | SEQ ID NO | SEQ ID NO |
| dVOYmiR- | 2 | 735 | 5 | 736 | 11 | 798 |
| 102.788 | | | | | | |
| dVOYmiR- | 2 | 737 | 5 | 738 | 11 | 799 |
| 102.805 | | | | | | |
| dVOYmiR- | 2 | 739 | 5 | 736 | 11 | 800 |
| 104.788 | | | | | | |
| dVOYmiR- | 2 | 740 | 5 | 738 | 11 | 801 |
| 104.805 | | | | | | |
| dVOYmiR- | 2 | 741 | 6 | 736 | 11 | 802 |
| 109.788 | | | | | | |
| dVOYmiR- | 2 | 742 | 6 | 738 | 11 | 803 |
| 109.805 | | | | | | |
| dVOYmiR- | 2 | 743 | 5 | 736 | 12 | 804 |
| 114.788 | | | | | | |
| dVOYmiR- | 2 | 744 | 5 | 738 | 12 | 805 |
| 114.805 | | | | | | |
| dVOYmiR- | 2 | 741 | 5 | 736 | 12 | 806 |
| 116.788 | | | | | | |
| dVOYmiR- | 2 | 742 | 5 | 738 | 12 | 807 |
| 116.805 | | | | | | |
| dVoymiR- | 3 | 741 | 9 | 745 | 14 | 808 |
| 127.788 | | | | | | |
| dVoymiR- | 3 | 742 | 9 | 746 | 14 | 809 |
| 127.805 | | | | | | |

### Example 15. Effect of the Position of Modulatory Polynucleotides

### A. Effect on Viral Titers

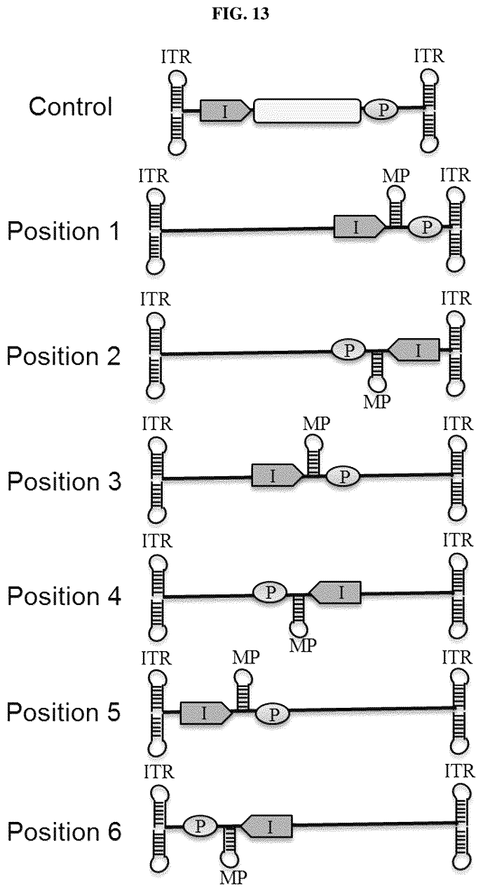

A modulatory polynucleotide (VOYmiR-114 or VOYmiR-126) was inserted into an expression vector (genome size approximately 2400 nucleotides; scAAV) at six different locations as shown in FIG. 13. In FIG. 13, “ITR” is the inverted terminal repeat, “I” represents intron, “P” is the polyA and “MP” is the modulatory polynucleotide. The viral titers were evaluated using TaqMan PCR for the 6 position and for a control (construct without a modulatory polynucleotide; scAAV) and the results are shown in Table 12.

TABLE 12
Viral Titers

| Modulatory | Modulatory | Virus Titer |
|---|---|---|
| Polynucleotide | Polynucleotide Position | (VG per 15-cm dish) |
| VOYmiR-114 | Position 1 | 5.5E+10 |
| VOYmiR-114 | Position 2 | 5.5E+10 |
| VOYmiR-114 | Position 3 | 4.5E+10 |
| VOYmiR-114 | Position 4 | 3.7E+10 |
| VOYmiR-114 | Position 5 | 6.5E+10 |
| VOYmiR-114 | Position 6 | 2.5E+10 |
| VOYmiR-126 | Position 1 | 1.6E+10 |
| VOYmiR-126 | Position 2 | 3.2E+10 |
| VOYmiR-126 | Position 3 | 6.0E+10 |
| VOYmiR-126 | Position 4 | 1.6E+10 |
| VOYmiR-126 | Position 5 | 9.5E+09 |
| VOYmiR-126 | Position 6 | 6.0E+10 |
| — | Control | 2.1E+11 |

### B. Effect on Genome Integrity

A modulatory polynucleotide (VOYmiR-114) was inserted into an expression vector (genome size 2400 nucleotides; scAAV) at six different locations and a control without a modulatory polynucleotide (scAAV) as shown in FIG. 13. In FIG. 13, “ITR” is the inverted terminal repeat, “I” represents intron, “P” is the polyA and “MP” is the modulatory polynucleotide. Viral genomes were extracted from purified AAV preparations and run on a neutral agarose gel. Truncated genomes were seen in all constructs and the approximate percent of the truncated genomes (percent of the total) is shown in Table 13.

TABLE 13
Truncated Genomes

| Construct | % of total |
|---|---|
| Position 1 | 50 |
| Position 2 | 42 |
| Position 3 | 49 |
| Position 4 | 34 |
| Position 5 | 33 |
| Position 6 | 59 |
| Control | 9 |

Position 6 had the greatest number of truncated genomes with Position 4 and 5 having the least amount of truncated genomes.

### C. Effect on Knock-Down Efficiency

A modulatory polynucleotide (VOYmiR-114) was inserted into an expression vector (AAV2) (genome size 2400 nucleotides; scAAV) at six different locations as shown in FIG. 13. In FIG. 13, “ITR” is the inverted terminal repeat, “I” represents intron, “P” is the polyA and “MP” is the modulatory polynucleotide. Transduction of HeLa cells was conducted at 1×10⁴ vg/cell, 1×10³ vg/cell and 1×10² vg/cell. The SOD1 mRNA expression (as % of control (eGFP)) was determined 72 hours post-infection and the results are shown in Table 14.

TABLE 14
SOD1 Expression

| | SOD1 mRNA expression (% of control) | | |
|---|---|---|---|
| Construct | 1 × 10⁴ vg/cell | 1 × 10³ vg/cell | 1 × 10² vg/cell |
| Position 1 | 40 | 59 | 69 |
| Position 2 | 31 | 46 | 75 |
| Position 3 | 50 | 66 | 81 |
| Position 4 | 21 | 34 | 55 |
| Position 5 | 49 | 52 | 67 |
| Position 6 | 31 | 37 | 62 |
| Control (eGFP) | 100 | 100 | 94 |

Position 3 had the highest SOD1 mRNA expression (as % of control) and Position 4 had the lowest SOD1 mRNA expression (as % of control).

### Example 16. Effect of Genome Size

### A. Effect on Viral Titers

A modulatory polynucleotide (VOYmiR-114) was inserted into an expression vector (genome size 2 kb; scAAV) at positions 1, 2, 5 and 6 as shown in FIG. 13. In FIG. 13, “ITR” is the inverted terminal repeat, “I” represents intron, “P” is the polyA and “MP” is the modulatory polynucleotide. A double stranded control without a modulatory polynucleotide (genome size 1.6 kb; scAAV ctrl) and a double stranded expression vector (scAAV miR114; ITR (105 nucleotide)-Promoter (˜900 nucleotides)-modulatory polynucleotide (158 nucleotides)-polyA sequence (127 nucleotides) and ITR) was compared as well as a control (eGFP; scAAV) with no modulatory polynucleotide. The viral titers were evaluated using TaqMan PCR and the results are shown in Table 15.

TABLE 15
Viral Titers

| Construct | Size | Virus Titer (VG per 15-cm dish) |
|---|---|---|
| Position 1 | 2 kb | 9.5E+10 |
| Position 2 | 2 kb | 1.2E+11 |
| scAAV miR114 | 1.6 kb | 1.1E+11 |
| Position 5 | 2 kb | 2.4E+10 |
| Position 6 | 2 kb | 1.1E+11 |
| Control | 2 kb | 2.2E+11 |

The lowest viral titers were seen with the position 5 construct and the greatest was with the position 2 construct.

### B. Effect on Genome Integrity

A modulatory polynucleotide (VOYmiR-114) was inserted into an expression vector (genome size 2 kb; scAAV) at positions 1, 2, 5 and 6 as shown in FIG. 13. In FIG. 13, “ITR” is the inverted terminal repeat, “I” represents intron, “P” is the polyA and “MP” is the modulatory polynucleotide. A double stranded control without a modulatory polynucleotide (genome size 1.6 kb; scAAV ctrl) and a double stranded expression vector (scAAV miR114; ITR (105 nucleotide)-Promoter (˜900 nucleotides)-modulatory polynucleotide (158 nucleotides)-polyA sequence (127 nucleotides) and ITR) was compared as well as a control (eGFP; scAAV) with no modulatory polynucleotide. Truncated genomes were seen in all constructs and the approximate percent of the truncated genomes (percent of the total) is shown in Table 16.

TABLE 16
Truncated Genomes

| Construct | Size | % of total |
|---|---|---|
| Position 1 | 2 kb | 34 |
| Position 2 | 2 kb | 30 |
| scAAV miR114 | 1.6 kb | 20 |
| Position 5 | 2 kb | 21 |
| Position 6 | 2 kb | 46 |
| Control | 2 kb | 5 |

All constructs were determined to have some truncated genomes.

An additional study was conducted to determine the effect of different modulatory polynucleotides. VOYmiR-114 and VOYmiR-126 were inserted into separate expression vectors (genome size 1.6 kb; scAAV) with the modulatory polynucleotide near the 3′ ITR (forward orientation). For the VOYmiR-114 construct the distance between the 5′ end of the vector genome (1526 nucleotides) and the center of the modulatory polynucleotide (middle of the flexible loop) is 1115 nucleotides. For the VOYmiR-126 construct the distance between the 5′ end of the vector genome (1626 nucleotides) and the center of the modulatory polynucleotide (middle of the flexible loop) is 1164 nucleotides.

For the VOYmiR-114 construct, the viral titer (VG per 15-cm dish) was about 1.1E+11. For the VOYmiR-126 construct, the intron probe viral titer (VG per 15-cm dish) was about 1.2E+12. The control was about 2.1E+11 (VG per 15-cm dish). VOYmir-114 had about 20% truncated genomes, VOYmiR-126 has about 15% truncated genomes and the control had about 5% truncated genomes.

### Example 17. Effect of Single Stranded Constructs

### A. Effect on Viral Titers

A modulatory polynucleotide (VOYmiR-114) was inserted into an expression vector (genome size 4.7 kb; ssAAV) at positions 1, 3 and 5 as shown in FIG. 13 and there was a control also tested without a modulatory polynucleotide (genome size 2 kb; ssAAV). In FIG. 13, “ITR” is the inverted terminal repeat, “I” represents intron, “P” is the polyA and “MP” is the modulatory polynucleotide. The viral titers were evaluated using TaqMan PCR and the results are shown in Table 17.

TABLE 17
Viral Titers

| Construct | Virus Titer (VG per 15-cm dish) |
|---|---|
| Position 1 | 5.0E+11 |
| Position 3 | 7.5E+11 |
| Position 5 | 3.5E+11 |
| Control | 2.5E+11 |

Position 3 showed the greatest viral titers followed by position 1 and then position 5.

### B. Effect on Genome Integrity

A modulatory polynucleotide (VOYmiR-114) was inserted into an expression vector (genome size 4.7 kb; ssAAV) at positions 1, 3 and 5 as shown in FIG. 13 and there was a control also tested without a modulatory polynucleotide (genome size 2 kb; ssAAV). In FIG. 13, “ITR” is the inverted terminal repeat, “I” represents intron, “P” is the polyA and “MP” is the modulatory polynucleotide. Viral genomes were extracted from purified AAV preparations and run on a neutral agarose gel. Truncated genomes were seen in all constructs and the approximate percent of the truncated genomes (percent of the total) is shown in Table 18.

TABLE 18
Truncated Genomes

| Construct | % of total |
|---|---|
| Position 1 | 48 |
| Position 3 | 30 |
| Position 5 | 72 |
| Control | 0 |

Position 5 had the greatest number of truncated genomes with Position 3 having the least amount of truncated genomes.

### C. Effect on Knock-Down Efficiency

A modulatory polynucleotide (VOYmiR-114) was inserted into an expression vector (genome size 4.7 kb; ssAAV) at positions 1, 3 and 5 as shown in FIG. 13 and there was a single stranded control without a modulatory polynucleotide (genome size 2 kb; ssAAV ctrl), a double stranded control without a modulatory polynucleotide (genome size 1.6 kb; scAAV ctrl) and a double stranded expression vector (scAAV miR114; ITR (105 nucleotide)-Promoter (˜900 nucleotides)-modulatory polynucleotide (158 nucleotides)-polyA sequence (127 nucleotides) and ITR). In FIG. 13, “ITR” is the inverted terminal repeat, “I” represents intron, “P” is the polyA and “MP” is the modulatory polynucleotide. Transduction of HeLa cells was conducted at 1×10⁴ vg/cell, 1×10³ vg/cell and 1×10² vg/cell. The SOD1 mRNA expression (as % of control (eGFP)) was determined 72 hours post-infection and the results are shown in Table 19.

TABLE 19
SOD1 Expression

| | SOD1 mRNA expression (% of control) | | |
|---|---|---|---|
| Construct | 1 × 10⁴ vg/cell | 1 × 10³ vg/cell | 1 × 10² vg/cell |
| Position 1 | 62 | 85 | 87 |
| Position 3 | 77 | 93 | 99 |
| Position 5 | 59 | 82 | 84 |
| ssAAV ctrl | 100 | 101 | 108 |
| scAAV ctrl | 95 | 97 | 102 |
| scAAV miR114 | 23 | 33 | 62 |

Position 3 had the highest SOD1 mRNA expression (as % of control), then position 1 and the single stranded constructs with the lowest SOD1 mRNA expression (as % of control) was Position 5. None of the single stranded constructs had knock-down efficiency that was as low as the double stranded control with a modulatory polynucleotide.

### Example 18. SOD1 Knock-Down In Vivo

To evaluate the in vivo biological activity of pri-miRNAs, self-complementary pri-miRNAs (VOYmiR-114.806, VOYmiR127.806, VOYmiR102.860, VOYmiR109.860, VOYmiR114.860, VOYmiR116.860, VOYmiR127.860, VOYmiR102.861, VOYmiR104.861, VOYmiR109.861, VOYmiR114.861, VOYmiR109.866, VOYmiR116.866, or VOYmiR127.866) are packaged in AAV-DJ with a CBA promoter.

In mice, these packaged pri-miRNAs or a control of vehicle only (phosphate-buffered saline with 5% sorbitol and 0.001% F-68) were administered by a 10 minute intrastriatal infusion. Female or male Tg(SOD1)3Cje/J mice (Jackson Laboratory, Bar Harbor, ME), which express human SOD1, and of approximately 20-30 g body weight, receive unilateral injections of 5 uL test article which is targeted to the striatum (anteroposterior+0.5 mm, mediolateral+2 mm, relative to bregma; dorsoventral 3.8 mm, relative to skull surface). Test articles are injected (5 animals per test article) at 0.5 uL/min. using pre-filled, pump-regulated Hamilton micro-syringes (1701 model, 10 μl) with 33 gauge needles. At 1, 2, 3, 4 or 6 weeks following the injection, animals are sacrificed, brains are removed, and ipsilateral striata encompassing the infusion site from a 1 mm coronal slab, as well as striatal tissue from the adjacent 1 mm coronal slabs are dissected and flash frozen. Mouse tissue samples are lysed, and human SOD1 protein levels, and SOD1 and mouse GAPDH (mGAPDH) mRNA levels are quantified. SOD1 protein levels are quantified by ELISA (eBioscience (Affymetrix, San Diego, CA)), and total protein levels are quantified by BCA analysis (ThermoFisher Scientific, Waltham, MA). For each tissue sample, the level of SOD1 protein normalized to total protein is calculated as an average of 2 determinations. These normalized SOD1 protein levels are further normalized to the vehicle group, then averaged to obtain a group (treatment) average. SOD1 and mGAPDH mRNA levels are quantified by qRT-PCR. For each tissue sample, the ratio of SOD1/mGAPDH (normalized SOD1 mRNA level) is calculated as an average of 3 determinations. These ratios are then averaged to obtain a group (treatment) average. These group averages are further normalized to the vehicle group.

In non-human primates, test articles (1×10¹³-3×10¹³ vg of pri-miRNA packaged in AAV-DJ with a CBA promoter) or vehicle are administered by intrathecal lumbar bolus. Female cynomolgus monkeys (Macaca fascicularis, CR Research Model Houston, Houston, TX) of approximately 2.5-8.5 kg body weight, receive implanted single intrathecal catheters with the tip of the catheter located at the lumbar spine. Test articles are administered (4 animals per test article) comprising three 1 mL bolus injections (1 mL/minute), at approximately 60 minute intervals. At 4 to 6 weeks following the administration, animals are sacrificed, and selected tissues harvested for bioanalytical and histological evaluation. SOD1 protein and mRNA levels are assessed for suppression after treatment with pri-miRNA packaged in AAV-DJ with a CBA promoter, relative to the vehicle group.

### Example 19. SOD1 Knock-Down In Vivo Using VOYmiR-114.806

In Tg(SOD1)3Cje/J mice, VOYmiR-114.806 packaged in AAVDJ with a CBA promoter is administered as described in Example 18. The mice were administered by unilateral intrastriatal administration a dose of 3.7×10⁹ vg. After 1 or 2 weeks, there was no significant reduction in normalized SOD1 protein levels; normalized SOD1 protein levels were 98±11% (standard deviation) and 98±10% of the vehicle control group after 1 and 2 weeks, respectively. By week 3, VOYmiR-114.806 reduced the normalized SOD1 protein level to 84±9.0% of the vehicle control group, which was statistically significant (p<0.05, One-way ANOVA with Dunnett's post-hoc analysis). By weeks 4 and 6, VOYmiR-114.806 reduced the normalized SOD1 protein level to 73±7.9% (p<0.0001) and 75±7.4% (p<0.0001), respectively, of the vehicle control group. These results demonstrate that VOYmiR-114.806 packaged in AAV-DJ with a CBA promoter, is efficacious in vivo in down-modulating SOD1 protein levels. In addition, these results demonstrate that a total intrastriatal dose as low as 3.7×10⁹ vg of VOYmiR-114.806 packaged in AAVDJ with a CBA promoter resulted in significant down-modulation of SOD1 protein levels.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1.-24. (canceled)

25. A modulatory polynucleotide comprising:
(i) a 5′ flanking region comprising the nucleotide sequence of any one of SEQ ID NOs: 1-4;
(ii) a loop region comprising the nucleotide sequence of any one of SEQ ID NOs: 5-9; and
(iii) a 3′ flanking region comprising the nucleotide sequence of any one of SEQ ID NOs: 10-12, 14, or 810.

26. The modulatory polynucleotide of claim 25, wherein:
(i) the 5′ flanking region comprises the nucleotide sequence of SEQ ID NO: 2 or SEQ ID NO: 3;
(ii) the loop region comprises the nucleotide sequence of SEQ ID NO: 5, SEQ ID NO: 6, or SEQ ID NO: 9; and
(ii) the 3′ flanking region comprises the nucleotide sequence of SEQ ID NO: 11 or SEQ ID NO: 12.

27. The modulatory polynucleotide of claim 25, wherein the 5′ flanking region comprises the nucleotide sequence of SEQ ID NO: 2, the loop region comprises the nucleotide sequence of SEQ ID NO: 5, and the 3′ flanking region comprises the nucleotide sequence of SEQ ID NO: 11.

28. The modulatory polynucleotide of claim 25, wherein the 5′ flanking region comprises the nucleotide sequence of SEQ ID NO: 2, the loop region comprises the nucleotide sequence of SEQ ID NO: 5, and the 3′ flanking region comprises the nucleotide sequence of SEQ ID NO: 12.

29. The modulatory polynucleotide of claim 25, wherein the 5′ flanking region comprises the nucleotide sequence of SEQ ID NO: 2, the loop region comprises the nucleotide sequence of SEQ ID NO: 6, and the 3′ flanking region comprises the nucleotide sequence of SEQ ID NO: 11.

30. The modulatory polynucleotide of claim 25, which further comprises a passenger strand and a guide strand.

31. The modulatory polynucleotide of claim 30, wherein the passenger strand is located between the 5′ flanking region and the loop region and the guide strand is located between the loop region and the 3′ flanking region.

32. The modulatory polynucleotide of claim 30, wherein the guide strand is located between the 5′ flanking region and the loop region and the passenger strand is located between the loop region and the 3′ flanking region.

33. The modulatory polynucleotide of claim 30, wherein:
(i) the guide strand is 15-30 or 21-22 nucleotides in length; or
(ii) the passenger strand is 15-30 or 21-22 nucleotides in length.

34. The modulatory polynucleotide of claim 30, wherein:
(i) the passenger strand is at least 70% complementary to the guide strand; or
(ii) the guide strand is at least 70% complementary to a target RNA, wherein the target RNA is expressed in a neurologic cell, tissue, or organ.

35. The modulatory polynucleotide of claim 25, wherein the modulatory polynucleotide inhibits the expression of a SOD1 gene.

36. A vector encoding the modulatory polynucleotide of claim 25.

37. An isolated cell comprising the modulatory polynucleotide of claim 25, which is a human cell, an HEK293 cell, a cell of the central nervous system, a motor neuron, or an astrocyte.

38. An adeno-associated virus (AAV) viral genome comprising a nucleotide sequence positioned between two inverted terminal repeats (ITRs), wherein the nucleotide sequence encodes the modulatory polynucleotide of claim 25.

39. The AAV viral genome of claim 38, which further comprises one, two, three or all of:
(i) a promoter operably linked to the nucleotide sequence encoding the modulatory polynucleotide;
(ii) an enhancer;
(iii) an intron region; or
(iv) a polyadenylation (polyA) signal region.

40. The AAV viral genome of claim 39, wherein the promoter is a ubiquitous promoter.

41. The AAV viral genome of claim 39, wherein the promoter is a tissue-specific promoter.

42. The AAV viral genome of claim 39, wherein the promoter is a chicken β-actin (CBA) promoter, an elongation factor 1α-subunit (EF1α) promoter, a synapsin (Syn) promoter, a methyl-CpG binding protein 2 (MeCP2) promoter, a glial fibrillary acidic protein (GFAP) promoter, a neuron-specific enolase (NSE) promoter, a myelin basic protein (MBP) promoter, a PGK promoter, a CAG promoter, a ubiquitin C (UBC) promoter, or a cytomegalovirus (CMV) promoter.

43. An adeno-associated virus (AAV) particle comprising the AAV viral genome of claim 38, and an AAV capsid protein.

44. The AAV particle of claim 43, wherein the AAV capsid protein comprises an AAV5 capsid protein or a variant thereof, or an AAV9 capsid protein or a variant thereof.

45. A pharmaceutical composition comprising the AAV particle of claim 43, and a pharmaceutically acceptable excipient.

46. A method of treating a disease of the central nervous system in a subject, comprising administering to the subject the AAV particle of claim 43, thereby treating the disease of the central nervous system in the subject.

47. The method of claim 46, wherein the AAV particle is administered intravenously, via intracisternal injection, intravascularly, intraventricularly, or a combination thereof.

48. The method of claim 46, wherein the disease of the central nervous system is Huntington's disease.

49. The method of claim 46, wherein the disease of the central nervous system is Amyotrophic Lateral Sclerosis (ALS).


SEQUENCE LISTING (810 sequences)

SEQ ID NO: 1 - RNA, 50 nt, synthetic construct
  misc_feature 1..50: note = Description of Artificial Sequence: Synthetic oligonucleotide
  source 1..50: mol_type = other RNA
tttatgcctcatcctctgagtgctgaaggcttgctgtaggctgtatgctg

SEQ ID NO: 2 - RNA, 54 nt, synthetic construct
  misc_feature 1..54: note = Description of Artificial Sequence: Synthetic oligonucleotide
  source 1..54: mol_type = other RNA
gtgctgggcggggggcggcgggccctcccgcagaacaccatgcgctcttcggaa

SEQ ID NO: 3 - RNA, 100 nt, synthetic construct
  misc_feature 1..100: note = Description of Artificial Sequence: Synthetic polynucleotide
  source 1..100: mol_type = other RNA
gaagcaaagaaggggcagagggagcccgtgagctgagtgggccagggactgggagaaggagtgaggaggcagggccggcatgcctctgctgctggccaga

SEQ ID NO: 4 - RNA, 54 nt, synthetic construct
  misc_feature 1..54: note = Description of Artificial Sequence: Synthetic oligonucleotide
  source 1..54: mol_type = other RNA
gtgctgggcggggggcggcgggccctcccgcagaacaccatgcgctcttcggga

SEQ ID NO: 5 - RNA, 10 nt, synthetic construct
  misc_feature 1..10: note = Description of Artificial Sequence: Synthetic oligonucleotide
  source 1..10: mol_type = other RNA
tgtgacctgg

SEQ ID NO: 6 - RNA, 10 nt, synthetic construct
  misc_feature 1..10: note = Description of Artificial Sequence: Synthetic oligonucleotide
  source 1..10: mol_type = other RNA
tgtgatttgg

SEQ ID NO: 7 - RNA, 10 nt, synthetic construct
  misc_feature 1..10: note = Description of Artificial Sequence: Synthetic oligonucleotide
  source 1..10: mol_type = other RNA
tataatttgg

SEQ ID NO: 8 - RNA, 11 nt, synthetic construct
  misc_feature 1..11: note = Description of Artificial Sequence: Synthetic oligonucleotide
  source 1..11: mol_type = other RNA
cctgacccagt

SEQ ID NO: 9 - RNA, 18 nt, synthetic construct
  misc_feature 1..18: note = Description of Artificial Sequence: Synthetic oligonucleotide
  source 1..18: mol_type = other RNA
gtctgcacctgtcactag

SEQ ID NO: 10 - RNA, 50 nt, synthetic construct
  misc_feature 1..50: note = Description of Artificial Sequence: Synthetic oligonucleotide
  source 1..50: mol_type = other RNA
agtgtatgatgcctgttactagcattcacatggaacaaattgctgccgtg

SEQ ID NO: 11 - RNA, 52 nt, synthetic construct
  misc_feature 1..52: note = Description of Artificial Sequence: Synthetic oligonucleotide
  source 1..52: mol_type = other RNA
ctgaggagcgccttgacagcagccatgggagggccgccccctacctcagtga

SEQ ID NO: 12 - RNA, 52 nt, synthetic construct
  misc_feature 1..52: note = Description of Artificial Sequence: Synthetic oligonucleotide
  source 1..52: mol_type = other RNA
ctgtggagcgccttgacagcagccatgggagggccgccccctacctcagtga

SEQ ID NO: 13 - RNA, 100 nt, synthetic construct
  misc_feature 1..100: note = Description of Artificial Sequence: Synthetic polynucleotide
  source 1..100: mol_type = other RNA
tggccgtgtagtgctacccagcgctggctgcctcctcagcattgcaattcctctcccatctgggcaccagtcagctaccctggtgggaatctgggtagcc

SEQ ID NO: 14 - RNA, 99 nt, synthetic construct
  misc_feature 1..99: note = Description of Artificial Sequence: Synthetic oligonucleotide
  source 1..99: mol_type = other RNA
ggccgtgtagtgctacccagcgctggctgcctcctcagcattgcaattcctctcccatctgggcaccagtcagctaccctggtgggaatctgggtagcc

SEQ ID NO: 15 - DNA, 981 bp, Homo sapiens
  source 1..981: mol_type = unassigned DNA
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 16 - RNA, 19 nt, synthetic construct
  misc_feature 1..19: note = Description of Artificial Sequence: Synthetic oligonucleotide
  source 1..19: mol_type = other RNA
cggaggtctggcctataaa

SEQ ID NO: 17 - RNA, 19 nt, synthetic construct
  misc_feature 1..19: note = Description of Artificial Sequence: Synthetic oligonucleotide
  source 1..19: mol_type = other RNA
tttataggccagacctccg

SEQ ID NO: 18 - RNA, 19 nt, synthetic construct
  misc_feature 1..19: note = Description of Artificial Sequence: Synthetic oligonucleotide
  source 1..19: mol_type = other RNA
ggaggtctggcctataaag

SEQ ID NO: 19 - RNA, 19 nt, synthetic construct
  misc_feature 1..19: note = Description of Artificial Sequence: Synthetic oligonucleotide
  source 1..19: mol_type = other RNA
ctttataggccagacctcc

SEQ ID NO: 20 - RNA, 19 nt, synthetic construct
  misc_feature 1..19: note = Description of Artificial Sequence: Synthetic oligonucleotide
  source 1..19: mol_type = other RNA
gaggtctggcctataaagt

SEQ ID NO: 21 - RNA, 19 nt, synthetic construct
  misc_feature 1..19: note = Description of Artificial Sequence: Synthetic oligonucleotide
  source 1..19: mol_type = other RNA
actttataggccagacctc

SEQ ID NO: 22 - RNA, 19 nt, synthetic construct
  misc_feature 1..19: note = Description of Artificial Sequence: Synthetic oligonucleotide
  source 1..19: mol_type = other RNA
aggtctggcctataaagta

SEQ ID NO: 23 - RNA, 19 nt, synthetic construct
  misc_feature 1..19: note = Description of Artificial Sequence: Synthetic oligonucleotide
  source 1..19: mol_type = other RNA
tactttataggccagacct

SEQ ID NO: 24 - RNA, 19 nt, synthetic construct
  misc_feature 1..19: note = Description of Artificial Sequence: Synthetic oligonucleotide
  source 1..19: mol_type = other RNA
ggtctggcctataaagtag

SEQ ID NO: 25 - RNA, 19 nt, synthetic construct
  misc_feature 1..19: note = Description of Artificial Sequence: Synthetic oligonucleotide
  source 1..19: mol_type = other RNA
ctactttataggccagacc

SEQ ID NO: 26 - RNA, 19 nt, synthetic construct
  misc_feature 1..19: note = Description of Artificial Sequence: Synthetic oligonucleotide
  source 1..19: mol_type = other RNA
tctggcctataaagtagtc

SEQ ID NO: 27 - RNA, 19 nt, synthetic construct
  misc_feature 1..19: note = Description of Artificial Sequence: Synthetic oligonucleotide
  source 1..19: mol_type = other RNA
gactactttataggccaga

SEQ ID NO: 28 - RNA, 19 nt, synthetic construct
  misc_feature 1..19: note = Description of Artificial Sequence: Synthetic oligonucleotide
  source 1..19: mol_type = other RNA
ctggcctataaagtagtcg

SEQ ID NO: 29 - RNA, 19 nt, synthetic construct
  misc_feature 1..19: note = Description of Artificial Sequence: Synthetic oligonucleotide
  source 1..19: mol_type = other RNA
cgactactttataggccag

SEQ ID NO: 30 - RNA, 19 nt, synthetic construct
  misc_feature 1..19: note = Description of Artificial Sequence: Synthetic oligonucleotide
  source 1..19: mol_type = other RNA
tggcctataaagtagtcgc

SEQ ID NO: 31 - RNA, 19 nt, synthetic construct
  misc_feature 1..19: note = Description of Artificial Sequence: Synthetic oligonucleotide
  source 1..19: mol_type = other RNA
gcgactactttataggcca

SEQ ID NO: 32 - RNA, 19 nt, synthetic construct
  misc_feature 1..19: note = Description of Artificial Sequence: Synthetic oligonucleotide
  source 1..19: mol_type = other RNA
ggcctataaagtagtcgcg

SEQ ID NO: 33 - RNA, 19 nt, synthetic construct
  misc_feature 1..19: note = Description of Artificial Sequence: Synthetic oligonucleotide
  source 1..19: mol_type = other RNA
cgcgactactttataggcc

SEQ ID NO: 34 - RNA, 19 nt, synthetic construct
  misc_feature 1..19: note = Description of Artificial Sequence: Synthetic oligonucleotide
  source 1..19: mol_type = other RNA
gcctataaagtagtcgcgg

SEQ ID NO: 35 - RNA, 19 nt, synthetic construct
  misc_feature 1..19: note = Description of Artificial Sequence: Synthetic oligonucleotide
  source 1..19: mol_type = other RNA
ccgcgactactttataggc

SEQ ID NO: 36 - RNA, 19 nt, synthetic construct
  misc_feature 1..19: note = Description of Artificial Sequence: Synthetic oligonucleotide
  source 1..19: mol_type = other RNA
cctataaagtagtcgcgga

SEQ ID NO: 37 - RNA, 19 nt, synthetic construct
  misc_feature 1..19: note = Description of Artificial Sequence: Synthetic oligonucleotide
  source 1..19: mol_type = other RNA
tccgcgactactttatagg

SEQ ID NO: 38 - RNA, 19 nt, synthetic construct
  misc_feature 1..19: note = Description of Artificial Sequence: Synthetic oligonucleotide
  source 1..19: mol_type = other RNA
gtcgtagtctcctgcagcg

SEQ ID NO: 39 - RNA, 19 nt, synthetic construct
  misc_feature 1..19: note = Description of Artificial Sequence: Synthetic oligonucleotide
  source 1..19: mol_type = other RNA
cgctgcaggagactacgac

SEQ ID NO: 40 - RNA, 19 nt, synthetic construct
  misc_feature 1..19: note = Description of Artificial Sequence: Synthetic oligonucleotide
  source 1..19: mol_type = other RNA
cgtagtctcctgcagcgtc

SEQ ID NO: 41 - RNA, 19 nt, synthetic construct
  misc_feature 1..19: note = Description of Artificial Sequence: Synthetic oligonucleotide
  source 1..19: mol_type = other RNA
gacgctgcaggagactacg

SEQ ID NO: 42 - RNA, 19 nt, synthetic construct
  misc_feature 1..19: note = Description of Artificial Sequence: Synthetic oligonucleotide
  source 1..19: mol_type = other RNA
gtagtctcctgcagcgtct

SEQ ID NO: 43 - RNA, 19 nt, synthetic construct
  misc_feature 1..19: note = Description of Artificial Sequence: Synthetic oligonucleotide
  source 1..19: mol_type = other RNA
agacgctgcaggagactac

SEQ ID NO: 44 - RNA, 19 nt, synthetic construct
  misc_feature 1..19: note = Description of Artificial Sequence: Synthetic oligonucleotide
  source 1..19: mol_type = other RNA
tagtctcctgcagcgtctg

SEQ ID NO: 45 - RNA, 19 nt, synthetic construct
  misc_feature 1..19: note = Description of Artificial Sequence: Synthetic oligonucleotide
  source 1..19: mol_type = other RNA
cagacgctgcaggagacta

SEQ ID NO: 46 - RNA, 19 nt, synthetic construct
  misc_feature 1..19: note = Description of Artificial Sequence: Synthetic oligonucleotide
  source 1..19: mol_type = other RNA
atggcgacgaaggccgtgt

SEQ ID NO: 47 - RNA, 19 nt, synthetic construct
  misc_feature 1..19: note = Description of Artificial Sequence: Synthetic oligonucleotide
  source 1..19: mol_type = other RNA
acacggccttcgtcgccat

SEQ ID NO: 48 - RNA, 19 nt, synthetic construct
  misc_feature 1..19: note = Description of Artificial Sequence: Synthetic oligonucleotide
  source 1..19: mol_type = other RNA
cgacgaaggccgtgtgcgt

SEQ ID NO: 49 - RNA, 19 nt, synthetic construct
  misc_feature 1..19: note = Description of Artificial Sequence: Synthetic oligonucleotide
  source 1..19: mol_type = other RNA
acgcacacggccttcgtcg

SEQ ID NO: 50 - RNA, 19 nt, synthetic construct
  misc_feature 1..19: note = Description of Artificial Sequence: Synthetic oligonucleotide
  source 1..19: mol_type = other RNA
gaaggccgtgtgcgtgctg

SEQ ID NO: 51 - RNA, 19 nt, synthetic construct
  misc_feature 1..19: note = Description of Artificial Sequence: Synthetic oligonucleotide
  source 1..19: mol_type = other RNA
cagcacgcacacggccttc

SEQ ID NO: 52 - RNA, 19 nt, synthetic construct
  misc_feature 1..19: note = Description of Artificial Sequence: Synthetic oligonucleotide
  source 1..19: mol_type = other RNA
ggccgtgtgcgtgctgaag

SEQ ID NO: 53 - RNA, 19 nt, synthetic construct
  misc_feature 1..19: note = Description of Artificial Sequence: Synthetic oligonucleotide
  source 1..19: mol_type = other RNA
cttcagcacgcacacggcc

SEQ ID NO: 54 - RNA, 19 nt, synthetic construct
  misc_feature 1..19: note = Description of Artificial Sequence: Synthetic oligonucleotide
  source 1..19: mol_type = other RNA
agggcgacggcccagtgca

SEQ ID NO: 55 - RNA, 19 nt, synthetic construct
  misc_feature 1..19: note = Description of Artificial Sequence: Synthetic oligonucleotide
  source 1..19: mol_type = other RNA
tgcactgggccgtcgccct

SEQ ID NO: 56 - RNA, 19 nt, synthetic construct
  misc_feature 1..19: note = Description of Artificial Sequence: Synthetic oligonucleotide
  source 1..19: mol_type = other RNA
tgcagggcatcatcaattt

SEQ ID NO: 57 - RNA, 19 nt, synthetic construct
  misc_feature 1..19: note = Description of Artificial Sequence: Synthetic oligonucleotide
  source 1..19: mol_type = other RNA
aaattgatgatgccctgca

SEQ ID NO: 58 - RNA, 19 nt, synthetic construct
  misc_feature 1..19: note = Description of Artificial Sequence: Synthetic oligonucleotide
  source 1..19: mol_type = other RNA
gcagggcatcatcaatttc

SEQ ID NO: 59 - RNA, 19 nt, synthetic construct
  misc_feature 1..19: note = Description of Artificial Sequence: Synthetic oligonucleotide
  source 1..19: mol_type = other RNA
gaaattgatgatgccctgc

SEQ ID NO: 60 - RNA, 19 nt, synthetic construct
  misc_feature 1..19: note = Description of Artificial Sequence: Synthetic oligonucleotide
  source 1..19: mol_type = other RNA
agggcatcatcaatttcga

SEQ ID NO: 61 - RNA, 19 nt, synthetic construct
  misc_feature 1..19: note = Description of Artificial Sequence: Synthetic oligonucleotide
  source 1..19: mol_type = other RNA
tcgaaattgatgatgccct

SEQ ID NO: 62 - RNA, 19 nt, synthetic construct
  misc_feature 1..19: note = Description of Artificial Sequence: Synthetic oligonucleotide
  source 1..19: mol_type = other RNA
gggcatcatcaatttcgag

SEQ ID NO: 63 - RNA, 19 nt, synthetic construct
  misc_feature 1..19: note = Description of Artificial Sequence: Synthetic oligonucleotide
  source 1..19: mol_type = other RNA
ctcgaaattgatgatgccc

SEQ ID NO: 64 - RNA, 19 nt, synthetic construct
  misc_feature 1..19: note = Description of Artificial Sequence: Synthetic oligonucleotide
  source 1..19: mol_type = other RNA
ggcatcatcaatttcgagc

SEQ ID NO: 65 - RNA, 19 nt, synthetic construct
  misc_feature 1..19: note = Description of Artificial Sequence: Synthetic oligonucleotide
  source 1..19: mol_type = other RNA
gctcgaaattgatgatgcc

SEQ ID NO: 66 - RNA, 19 nt, synthetic construct
  misc_feature 1..19: note = Description of Artificial Sequence: Synthetic oligonucleotide
  source 1..19: mol_type = other RNA
gcatcatcaatttcgagca

SEQ ID NO: 67 - RNA, 19 nt, synthetic construct
  misc_feature 1..19: note = Description of Artificial Sequence: Synthetic oligonucleotide
  source 1..19: mol_type = other RNA
tgctcgaaattgatgatgc

SEQ ID NO: 68 - RNA, 19 nt, synthetic construct
  misc_feature 1..19: note = Description of Artificial Sequence: Synthetic oligonucleotide
  source 1..19: mol_type = other RNA
catcatcaatttcgagcag

SEQ ID NO: 69 - RNA, 19 nt, synthetic construct
  misc_feature 1..19: note = Description of Artificial Sequence: Synthetic oligonucleotide
  source 1..19: mol_type = other RNA
ctgctcgaaattgatgatg

SEQ ID NO: 70 - RNA, 19 nt, synthetic construct
  misc_feature 1..19: note = Description of Artificial Sequence: Synthetic oligonucleotide
  source 1..19: mol_type = other RNA
aatttcgagcagaaggaaa

SEQ ID NO: 71 - RNA, 19 nt, synthetic construct
  misc_feature 1..19: note = Description of Artificial Sequence: Synthetic oligonucleotide
  source 1..19: mol_type = other RNA
tttccttctgctcgaaatt

SEQ ID NO: 72 - RNA, 19 nt, synthetic construct
  misc_feature 1..19: note = Description of Artificial Sequence: Synthetic oligonucleotide
  source 1..19: mol_type = other RNA
ttcgagcagaaggaaagta

SEQ ID NO: 73 - RNA, 19 nt, synthetic construct
  misc_feature 1..19: note = Description of Artificial Sequence: Synthetic oligonucleotide
  source 1..19: mol_type = other RNA
tactttccttctgctcgaa

SEQ ID NO: 74 - RNA, 19 nt, synthetic construct
  misc_feature 1..19: note = Description of Artificial Sequence: Synthetic oligonucleotide
  source 1..19: mol_type = other RNA
tcgagcagaaggaaagtaa

SEQ ID NO: 75 - RNA, 19 nt, synthetic construct
  misc_feature 1..19: note = Description of Artificial Sequence: Synthetic oligonucleotide
  source 1..19: mol_type = other RNA
ttactttccttctgctcga

SEQ ID NO: 76 - RNA, 19 nt, synthetic construct
  misc_feature 1..19: note = Description of Artificial Sequence: Synthetic oligonucleotide
  source 1..19: mol_type = other RNA
aaggtgtggggaagcatta

SEQ ID NO: 77 - RNA, 19 nt, synthetic construct
  misc_feature 1..19: note = Description of Artificial Sequence: Synthetic oligonucleotide
  source 1..19: mol_type = other RNA
taatgcttccccacacctt

SEQ ID NO: 78 - RNA, 19 nt, synthetic construct
  misc_feature 1..19: note = Description of Artificial Sequence: Synthetic oligonucleotide
  source 1..19: mol_type = other RNA
ggtgtggggaagcattaaa

SEQ ID NO: 79 - RNA, 19 nt, synthetic construct
  misc_feature 1..19: note = Description of Artificial Sequence: Synthetic oligonucleotide
  source 1..19: mol_type = other RNA
tttaatgcttccccacacc

SEQ ID NO: 80 - RNA, 19 nt, synthetic construct
  misc_feature 1..19: note = Description of Artificial Sequence: Synthetic oligonucleotide
  source 1..19: mol_type = other RNA
gactgactgaaggcctgca

SEQ ID NO: 81 - RNA, 19 nt, synthetic construct
  misc_feature 1..19: note = Description of Artificial Sequence: Synthetic oligonucleotide
  source 1..19: mol_type = other RNA
tgcaggccttcagtcagtc

SEQ ID NO: 82 - RNA, 19 nt, synthetic construct
  misc_feature 1..19: note = Description of Artificial Sequence: Synthetic oligonucleotide
  source 1..19: mol_type = other RNA
ctgactgaaggcctgcatg

SEQ ID NO: 83 - RNA, 19 nt, synthetic construct
  misc_feature 1..19: note = Description of Artificial Sequence: Synthetic oligonucleotide
  source 1..19: mol_type = other RNA
catgcaggccttcagtcag

SEQ ID NO: 84 - RNA, 19 nt, synthetic construct
  misc_feature 1..19: note = Description of Artificial Sequence: Synthetic oligonucleotide
  source 1..19: mol_type = other RNA
tgactgaaggcctgcatgg

SEQ ID NO: 85 - RNA, 19 nt, synthetic construct
  misc_feature 1..19: note = Description of Artificial Sequence: Synthetic oligonucleotide
  source 1..19: mol_type = other RNA
ccatgcaggccttcagtca

SEQ ID NO: 86 - RNA, 19 nt, synthetic construct
  misc_feature 1..19: note = Description of Artificial Sequence: Synthetic oligonucleotide
  source 1..19: mol_type = other RNA
tgaaggcctgcatggattc

SEQ ID NO: 87 - RNA, 19 nt, synthetic construct
  misc_feature 1..19: note = Description of Artificial Sequence: Synthetic oligonucleotide
  source 1..19: mol_type = other RNA
gaatccatgcaggccttca

SEQ ID NO: 88 - RNA, 19 nt, synthetic construct
  misc_feature 1..19: note = Description of Artificial Sequence: Synthetic oligonucleotide
  source 1..19: mol_type = other RNA
gaaggcctgcatggattcc

SEQ ID NO: 89 - RNA, 19 nt, synthetic construct
  misc_feature 1..19: note = Description of Artificial Sequence: Synthetic oligonucleotide
  source 1..19: mol_type = other RNA
ggaatccatgcaggccttc

SEQ ID NO: 90 - RNA, 19 nt, synthetic construct
  misc_feature 1..19: note = Description of Artificial Sequence: Synthetic oligonucleotide
  source 1..19: mol_type = other RNA
tgcatggattccatgttca

SEQ ID NO: 91 - RNA, 19 nt, synthetic construct
  misc_feature 1..19: note = Description of Artificial Sequence: Synthetic oligonucleotide
  source 1..19: mol_type = other RNA
tgaacatggaatccatgca

SEQ ID NO: 92 - RNA, 19 nt, synthetic construct
  misc_feature 1..19: note = Description of Artificial Sequence: Synthetic oligonucleotide
  source 1..19: mol_type = other RNA
catggattccatgttcatg

SEQ ID NO: 93 - RNA, 19 nt, synthetic construct
  misc_feature 1..19: note = Description of Artificial Sequence: Synthetic oligonucleotide
  source 1..19: mol_type = other RNA
catgaacatggaatccatg

SEQ ID NO: 94 - RNA, 19 nt, synthetic construct
  misc_feature 1..19: note = Description of Artificial Sequence: Synthetic oligonucleotide
  source 1..19: mol_type = other RNA
ggattccatgttcatgagt

SEQ ID NO: 95 - RNA, 19 nt, synthetic construct
  misc_feature 1..19: note = Description of Artificial Sequence: Synthetic oligonucleotide
  source 1..19: mol_type = other RNA
actcatgaacatggaatcc

SEQ ID NO: 96 - RNA, 19 nt, synthetic construct
  misc_feature 1..19: note = Description of Artificial Sequence: Synthetic oligonucleotide
  source 1..19: mol_type = other RNA
ttccatgttcatgagtttg

SEQ ID NO: 97 - RNA, 19 nt, synthetic construct
  misc_feature 1..19: note = Description of Artificial Sequence: Synthetic oligonucleotide
  source 1..19: mol_type = other RNA
caaactcatgaacatggaa

SEQ ID NO: 98 - RNA, 19 nt, synthetic construct
  misc_feature 1..19: note = Description of Artificial Sequence: Synthetic oligonucleotide
  source 1..19: mol_type = other RNA
gttcatgagtttggagata

SEQ ID NO: 99 - RNA, 19 nt, synthetic construct
  misc_feature 1..19: note = Description of Artificial Sequence: Synthetic oligonucleotide
  source 1..19: mol_type = other RNA
tatctccaaactcatgaac

SEQ ID NO: 100 - RNA, 19 nt, synthetic construct
  misc_feature 1..19: note = Description of Artificial Sequence: Synthetic oligonucleotide
  source 1..19: mol_type = other RNA
ttcatgagtttggagataa

SEQ ID NO: 101 - RNA, 19 nt, synthetic construct
  misc_feature 1..19: note = Description of Artificial Sequence: Synthetic oligonucleotide
  source 1..19: mol_type = other RNA
ttatctccaaactcatgaa

SEQ ID NO: 102 - RNA, 19 nt, synthetic construct
  misc_feature 1..19: note = Description of Artificial Sequence: Synthetic oligonucleotide
  source 1..19: mol_type = other RNA
tgagtttggagataataca

SEQ ID NO: 103 - RNA, 19 nt, synthetic construct
  misc_feature 1..19: note = Description of Artificial Sequence: Synthetic oligonucleotide
  source 1..19: mol_type = other RNA
tgtattatctccaaactca

SEQ ID NO: 104 - RNA, 19 nt, synthetic construct
  misc_feature 1..19: note = Description of Artificial Sequence: Synthetic oligonucleotide
  source 1..19: mol_type = other RNA
gagtttggagataatacag

SEQ ID NO: 105 - RNA, 19 nt, synthetic construct
  misc_feature 1..19: note = Description of Artificial Sequence: Synthetic oligonucleotide
  source 1..19: mol_type = other RNA
ctgtattatctccaaactc

SEQ ID NO: 106 - RNA, 19 nt, synthetic construct
  misc_feature 1..19: note = Description of Artificial Sequence: Synthetic oligonucleotide
  source 1..19: mol_type = other RNA
aggctgtaccagtgcaggt

SEQ ID NO: 107 - RNA, 19 nt, synthetic construct
  misc_feature 1..19: note = Description of Artificial Sequence: Synthetic oligonucleotide
  source 1..19: mol_type = other RNA
acctgcactggtacagcct

SEQ ID NO: 108 - RNA, 19 nt, synthetic construct
  misc_feature 1..19: note = Description of Artificial Sequence: Synthetic oligonucleotide
  source 1..19: mol_type = other RNA
ggctgtaccagtgcaggtc

SEQ ID NO: 109 - RNA, 19 nt, synthetic construct
  misc_feature 1..19: note = Description of Artificial Sequence: Synthetic oligonucleotide
  source 1..19: mol_type = other RNA
gacctgcactggtacagcc

SEQ ID NO: 110 - RNA, 19 nt, synthetic construct
  misc_feature 1..19: note = Description of Artificial Sequence: Synthetic oligonucleotide
  source 1..19: mol_type = other RNA
gcaggtcctcactttaatc

SEQ ID NO: 111 - RNA, 19 nt, synthetic construct
  misc_feature 1..19: note = Description of Artificial Sequence: Synthetic oligonucleotide
  source 1..19: mol_type = other RNA
gattaaagtgaggacctgc

SEQ ID NO: 112 - RNA, 19 nt, synthetic construct
  misc_feature 1..19: note = Description of Artificial Sequence: Synthetic oligonucleotide
  source 1..19: mol_type = other RNA
caggtcctcactttaatcc

SEQ ID NO: 113 - RNA, 19 nt, synthetic construct
  misc_feature 1..19: note = Description of Artificial Sequence: Synthetic oligonucleotide
  source 1..19: mol_type = other RNA
ggattaaagtgaggacctg

SEQ ID NO: 114 - RNA, 19 nt, synthetic construct
  misc_feature 1..19: note = Description of Artificial Sequence: Synthetic oligonucleotide
  source 1..19: mol_type = other RNA
tcactttaatcctctatcc

SEQ ID NO: 115 - RNA, 19 nt, synthetic construct
  misc_feature 1..19: note = Description of Artificial Sequence: Synthetic oligonucleotide
  source 1..19: mol_type = other RNA
ggatagaggattaaagtga

SEQ ID NO: 116 - RNA, 19 nt, synthetic construct
  misc_feature 1..19: note = Description of Artificial Sequence: Synthetic oligonucleotide
  source 1..19: mol_type = other RNA
ctatccagaaaacacggtg

SEQ ID NO: 117 - RNA, 19 nt, synthetic construct
  misc_feature 1..19: note = Description of Artificial Sequence: Synthetic oligonucleotide
  source 1..19: mol_type = other RNA
caccgtgttttctggatag

SEQ ID NO: 118 - RNA, 19 nt, synthetic construct
  misc_feature 1..19: note = Description of Artificial Sequence: Synthetic oligonucleotide
  source 1..19: mol_type = other RNA
tatccagaaaacacggtgg

SEQ ID NO: 119 - RNA, 19 nt, synthetic construct
  misc_feature 1..19: note = Description of Artificial Sequence: Synthetic oligonucleotide
  source 1..19: mol_type = other RNA
ccaccgtgttttctggata

SEQ ID NO: 120 - RNA, 19 nt, synthetic construct
  misc_feature 1..19: note = Description of Artificial Sequence: Synthetic oligonucleotide
  source 1..19: mol_type = other RNA
atccagaaaacacggtggg

SEQ ID NO: 121 - RNA, 19 nt, synthetic construct
  misc_feature 1..19: note = Description of Artificial Sequence: Synthetic oligonucleotide
  source 1..19: mol_type = other RNA
cccaccgtgttttctggat

SEQ ID NO: 122 - RNA, 19 nt, synthetic construct
  misc_feature 1..19: note = Description of Artificial Sequence: Synthetic oligonucleotide
  source 1..19: mol_type = other RNA
ccagaaaacacggtgggcc

SEQ ID NO: 123 - RNA, 19 nt, synthetic construct
  misc_feature 1..19: note = Description of Artificial Sequence: Synthetic oligonucleotide
  source 1..19: mol_type = other RNA
ggcccaccgtgttttctgg

SEQ ID NO: 124 - RNA, 19 nt, synthetic construct
  misc_feature 1..19: note = Description of Artificial Sequence: Synthetic oligonucleotide
  source 1..19: mol_type = other RNA
gaaaacacggtgggccaaa

SEQ ID NO: 125 - RNA, 19 nt, synthetic construct
  misc_feature 1..19: note = Description of Artificial Sequence: Synthetic oligonucleotide
  source 1..19: mol_type = other RNA
tttggcccaccgtgttttc

SEQ ID NO: 126 - RNA, 19 nt, synthetic construct
  misc_feature 1..19: note = Description of Artificial Sequence: Synthetic oligonucleotide
  source 1..19: mol_type = other RNA
aaaacacggtgggccaaag

SEQ ID NO: 127 - RNA, 19 nt, synthetic construct
  misc_feature 1..19: note = Description of Artificial Sequence: Synthetic oligonucleotide
  source 1..19: mol_type = other RNA
ctttggcccaccgtgtttt

SEQ ID NO: 128 - RNA, 19 nt, synthetic construct
  misc_feature 1..19: note = Description of Artificial Sequence: Synthetic oligonucleotide
  source 1..19: mol_type = other RNA
cggtgggccaaaggatgaa

SEQ ID NO: 129 - RNA, 19 nt, synthetic construct
  misc_feature 1..19: note = Description of Artificial Sequence: Synthetic oligonucleotide
  source 1..19: mol_type = other RNA
ttcatcctttggcccaccg

SEQ ID NO: 130 - RNA, 19 nt, synthetic construct
  misc_feature 1..19: note = Description of Artificial Sequence: Synthetic oligonucleotide
  source 1..19: mol_type = other RNA
aggatgaagagaggcatgt

SEQ ID NO: 131 - RNA, 19 nt, synthetic construct
  misc_feature 1..19: note = Description of Artificial Sequence: Synthetic oligonucleotide
  source 1..19: mol_type = other RNA
acatgcctctcttcatcct

SEQ ID NO: 132 - RNA, 19 nt, synthetic construct
  misc_feature 1..19: note = Description of Artificial Sequence: Synthetic oligonucleotide
  source 1..19: mol_type = other RNA
atgaagagaggcatgttgg

SEQ ID NO: 133 - RNA, 19 nt, synthetic construct
  misc_feature 1..19: note = Description of Artificial Sequence: Synthetic oligonucleotide
  source 1..19: mol_type = other RNA
ccaacatgcctctcttcat

SEQ ID NO: 134 - RNA, 19 nt, synthetic construct
  misc_feature 1..19: note = Description of Artificial Sequence: Synthetic oligonucleotide
  source 1..19: mol_type = other RNA
gagaggcatgttggagact

SEQ ID NO: 135 - RNA, 19 nt, synthetic construct
  misc_feature 1..19: note = Description of Artificial Sequence: Synthetic oligonucleotide
  source 1..19: mol_type = other RNA
agtctccaacatgcctctc

SEQ ID NO: 136 - RNA, 19 nt, synthetic construct
  misc_feature 1..19: note = Description of Artificial Sequence: Synthetic oligonucleotide
  source 1..19: mol_type = other RNA
agaggcatgttggagactt

SEQ ID NO: 137 - RNA, 19 nt, synthetic construct
  misc_feature 1..19: note = Description of Artificial Sequence: Synthetic oligonucleotide
  source 1..19: mol_type = other RNA
aagtctccaacatgcctct

SEQ ID NO: 138 - RNA, 19 nt, synthetic construct
  misc_feature 1..19: note = Description of Artificial Sequence: Synthetic oligonucleotide
  source 1..19: mol_type = other RNA
atgttggagacttgggcaa

SEQ ID NO: 139 - RNA, 19 nt, synthetic construct
  misc_feature 1..19: note = Description of Artificial Sequence: Synthetic oligonucleotide
  source 1..19: mol_type = other RNA
ttgcccaagtctccaacat

SEQ ID NO: 140 - RNA, 19 nt, synthetic construct
  misc_feature 1..19: note = Description of Artificial Sequence: Synthetic oligonucleotide
  source 1..19: mol_type = other RNA
gttggagacttgggcaatg

SEQ ID NO: 141 - RNA, 19 nt, synthetic construct
  misc_feature 1..19: note = Description of Artificial Sequence: Synthetic oligonucleotide
  source 1..19: mol_type = other RNA
cattgcccaagtctccaac

SEQ ID NO: 142 - RNA, 19 nt, synthetic construct
  misc_feature 1..19: note = Description of Artificial Sequence: Synthetic oligonucleotide
  source 1..19: mol_type = other RNA
ggagacttgggcaatgtga

SEQ ID NO: 143 - RNA, 19 nt, synthetic construct
  misc_feature 1..19: note = Description of Artificial Sequence: Synthetic oligonucleotide
  source 1..19: mol_type = other RNA
tcacattgcccaagtctcc

SEQ ID NO: 144 - RNA, 19 nt, synthetic construct
  misc_feature 1..19: note = Description of Artificial Sequence: Synthetic oligonucleotide
  source 1..19: mol_type = other RNA
ggcaatgtgactgctgaca

SEQ ID NO: 145 - RNA, 19 nt, synthetic construct
  misc_feature 1..19: note = Description of Artificial Sequence: Synthetic oligonucleotide
  source 1..19: mol_type = other RNA
tgtcagcagtcacattgcc

SEQ ID NO: 146 - RNA, 19 nt, synthetic construct
  misc_feature 1..19: note = Description of Artificial Sequence: Synthetic oligonucleotide
  source 1..19: mol_type = other RNA
caatgtgactgctgacaaa

SEQ ID NO: 147 - RNA, 19 nt, synthetic construct
  misc_feature 1..19: note = Description of Artificial Sequence: Synthetic oligonucleotide
  source 1..19: mol_type = other RNA
tttgtcagcagtcacattg

SEQ ID NO: 148 - RNA, 19 nt, synthetic construct
  misc_feature 1..19: note = Description of Artificial Sequence: Synthetic oligonucleotide
  source 1..19: mol_type = other RNA
ctgacaaagatggtgtggc

SEQ ID NO: 149 - RNA, 19 nt, synthetic construct
  misc_feature 1..19: note = Description of Artificial Sequence: Synthetic oligonucleotide
  source 1..19: mol_type = other RNA
gccacaccatctttgtcag

SEQ ID NO: 150 - RNA, 19 nt, synthetic construct
  misc_feature 1..19: note = Description of Artificial Sequence: Synthetic oligonucleotide
  source 1..19: mol_type = other RNA
tgacaaagatggtgtggcc

SEQ ID NO: 151 - RNA, 19 nt, synthetic construct
  misc_feature 1..19: note = Description of Artificial Sequence: Synthetic oligonucleotide
  source 1..19: mol_type = other RNA
ggccacaccatctttgtca

SEQ ID NO: 152 - RNA, 19 nt, synthetic construct
  misc_feature 1..19: note = Description of Artificial Sequence: Synthetic oligonucleotide
  source 1..19: mol_type = other RNA
ctcaggagaccattgcatc

SEQ ID NO: 153 - RNA, 19 nt, synthetic construct
  misc_feature 1..19: note = Description of Artificial Sequence: Synthetic oligonucleotide
  source 1..19: mol_type = other RNA
gatgcaatggtctcctgag

SEQ ID NO: 154 - RNA, 19 nt, synthetic construct
  misc_feature 1..19: note = Description of Artificial Sequence: Synthetic oligonucleotide
  source 1..19: mol_type = other RNA
tcaggagaccattgcatca

SEQ ID NO: 155 - RNA, 19 nt, synthetic construct
  misc_feature 1..19: note = Description of Artificial Sequence: Synthetic oligonucleotide
  source 1..19: mol_type = other RNA
tgatgcaatggtctcctga

SEQ ID NO: 156 - RNA, 19 nt, synthetic construct
  misc_feature 1..19: note = Description of Artificial Sequence: Synthetic oligonucleotide
  source 1..19: mol_type = other RNA
agaccattgcatcattggc

SEQ ID NO: 157 - RNA, 19 nt, synthetic construct
  misc_feature 1..19: note = Description of Artificial Sequence: Synthetic oligonucleotide
  source 1..19: mol_type = other RNA
gccaatgatgcaatggtct

SEQ ID NO: 158 - RNA, 19 nt, synthetic construct
  misc_feature 1..19: note = Description of Artificial Sequence: Synthetic oligonucleotide
  source 1..19: mol_type = other RNA
gaccattgcatcattggcc

SEQ ID NO: 159 - RNA, 19 nt, synthetic construct
  misc_feature 1..19: note = Description of Artificial Sequence: Synthetic oligonucleotide
  source 1..19: mol_type = other RNA
ggccaatgatgcaatggtc

SEQ ID NO: 160 - RNA, 19 nt, synthetic construct
  misc_feature 1..19: note = Description of Artificial Sequence: Synthetic oligonucleotide
  source 1..19: mol_type = other RNA
attgcatcattggccgcac

SEQ ID NO: 161 - RNA, 19 nt, synthetic construct
  misc_feature 1..19: note = Description of Artificial Sequence: Synthetic oligonucleotide
  source 1..19: mol_type = other RNA
gtgcggccaatgatgcaat

SEQ ID NO: 162 - RNA, 19 nt, synthetic construct
  misc_feature 1..19: note = Description of Artificial Sequence: Synthetic oligonucleotide
  source 1..19: mol_type = other RNA
cattggccgcacactggtg

SEQ ID NO: 163 - RNA, 19 nt, synthetic construct
  misc_feature 1..19: note = Description of Artificial Sequence: Synthetic oligonucleotide
  source 1..19: mol_type = other RNA
caccagtgtgcggccaatg

SEQ ID NO: 164 - RNA, 19 nt, synthetic construct
  misc_feature 1..19: note = Description of Artificial Sequence: Synthetic oligonucleotide
  source 1..19: mol_type = other RNA
cgcacactggtggtccatg

SEQ ID NO: 165 - RNA, 19 nt, synthetic construct
  misc_feature 1..19: note = Description of Artificial Sequence: Synthetic oligonucleotide
  source 1..19: mol_type = other RNA
catggaccaccagtgtgcg

SEQ ID NO: 166 - RNA, 19 nt, synthetic construct
  misc_feature 1..19: note = Description of Artificial Sequence: Synthetic oligonucleotide
  source 1..19: mol_type = other RNA
cacactggtggtccatgaa

SEQ ID NO: 167 - RNA, 19 nt, synthetic construct
  misc_feature 1..19: note = Description of Artificial Sequence: Synthetic oligonucleotide
  source 1..19: mol_type = other RNA
ttcatggaccaccagtgtg

SEQ ID NO: 168 - RNA, 19 nt, synthetic construct
  misc_feature 1..19: note = Description of Artificial Sequence: Synthetic oligonucleotide
  source 1..19: mol_type = other RNA
acactggtggtccatgaaa

SEQ ID NO: 169 - RNA, 19 nt, synthetic construct
  misc_feature 1..19: note = Description of Artificial Sequence: Synthetic oligonucleotide
  source 1..19: mol_type = other RNA
tttcatggaccaccagtgt

SEQ ID NO: 170 - RNA, 19 nt, synthetic construct
  misc_feature 1..19: note = Description of Artificial Sequence: Synthetic oligonucleotide
  source 1..19: mol_type = other RNA
tggtggtccatgaaaaagc

SEQ ID NO: 171 - RNA, 19 nt, synthetic construct
  misc_feature 1..19: note = Description of Artificial Sequence: Synthetic oligonucleotide
  source 1..19: mol_type = other RNA
gctttttcatggaccacca

SEQ ID NO: 172 - RNA, 19 nt, synthetic construct
  misc_feature 1..19: note = Description of Artificial Sequence: Synthetic oligonucleotide
  source 1..19: mol_type = other RNA
tggtccatgaaaaagcaga

SEQ ID NO: 173 - RNA, 19 nt, synthetic construct
  misc_feature 1..19: note = Description of Artificial Sequence: Synthetic oligonucleotide
  source 1..19: mol_type = other RNA
tctgctttttcatggacca

SEQ ID NO: 174 - RNA, 19 nt, synthetic construct
  misc_feature 1..19: note = Description of Artificial Sequence: Synthetic oligonucleotide
  source 1..19: mol_type = other RNA
aaagcagatgacttgggca

SEQ ID NO: 175 - RNA, 19 nt, synthetic construct
  misc_feature 1..19: note = Description of Artificial Sequence: Synthetic oligonucleotide
  source 1..19: mol_type = other RNA
tgcccaagtcatctgcttt

SEQ ID NO: 176 - RNA, 19 nt, synthetic construct
  misc_feature 1..19: note = Description of Artificial Sequence: Synthetic oligonucleotide
  source 1..19: mol_type = other RNA
gcagatgacttgggcaaag

SEQ ID NO: 177 - RNA, 19 nt, synthetic construct
  misc_feature 1..19: note = Description of Artificial Sequence: Synthetic oligonucleotide
  source 1..19: mol_type = other RNA
ctttgcccaagtcatctgc

SEQ ID NO: 178 - RNA, 19 nt, synthetic construct
  misc_feature 1..19: note = Description of Artificial Sequence: Synthetic oligonucleotide
  source 1..19: mol_type = other RNA
atgacttgggcaaaggtgg

SEQ ID NO: 179 - RNA, 19 nt, synthetic construct
  misc_feature 1..19: note = Description of Artificial Sequence: Synthetic oligonucleotide
  source 1..19: mol_type = other RNA
ccacctttgcccaagtcat

SEQ ID NO: 180 - RNA, 19 nt, synthetic construct
  misc_feature 1..19: note = Description of Artificial Sequence: Synthetic oligonucleotide
  source 1..19: mol_type = other RNA
tgacttgggcaaaggtgga

SEQ ID NO: 181 - RNA, 19 nt, synthetic construct
  misc_feature 1..19: note = Description of Artificial Sequence: Synthetic oligonucleotide
  source 1..19: mol_type = other RNA
tccacctttgcccaagtca

SEQ ID NO: 182 - RNA, 19 nt, synthetic construct
  misc_feature 1..19: note = Description of Artificial Sequence: Synthetic oligonucleotide
  source 1..19: mol_type = other RNA
gacttgggcaaaggtggaa

SEQ ID NO: 183 - RNA, 19 nt, synthetic construct
  misc_feature 1..19: note = Description of Artificial Sequence: Synthetic oligonucleotide
  source 1..19: mol_type = other RNA
ttccacctttgcccaagtc

SEQ ID NO: 184 - RNA, 19 nt, synthetic construct
  misc_feature 1..19: note = Description of Artificial Sequence: Synthetic oligonucleotide
  source 1..19: mol_type = other RNA
gtacaaagacaggaaacgc

SEQ ID NO: 185 - RNA, 19 nt, synthetic construct
  misc_feature 1..19: note = Description of Artificial Sequence: Synthetic oligonucleotide
  source 1..19: mol_type = other RNA
gcgtttcctgtctttgtac

SEQ ID NO: 186 - RNA, 19 nt, synthetic construct
  misc_feature 1..19: note = Description of Artificial Sequence: Synthetic oligonucleotide
  source 1..19: mol_type = other RNA
acaaagacaggaaacgctg

SEQ ID NO: 187 - RNA, 19 nt, synthetic construct
  misc_feature 1..19: note = Description of Artificial Sequence: Synthetic oligonucleotide
  source 1..19: mol_type = other RNA
cagcgtttcctgtctttgt

SEQ ID NO: 188 - RNA, 19 nt, synthetic construct
  misc_feature 1..19: note = Description of Artificial Sequence: Synthetic oligonucleotide
  source 1..19: mol_type = other RNA
caaagacaggaaacgctgg

SEQ ID NO: 189 - RNA, 19 nt, synthetic construct
  misc_feature 1..19: note = Description of Artificial Sequence: Synthetic oligonucleotide
  source 1..19: mol_type = other RNA
ccagcgtttcctgtctttg

SEQ ID NO: 190 - RNA, 19 nt, synthetic construct
  misc_feature 1..19: note = Description of Artificial Sequence: Synthetic oligonucleotide
  source 1..19: mol_type = other RNA
aggaaacgctggaagtcgt

SEQ ID NO: 191 - RNA, 19 nt, synthetic construct
  misc_feature 1..19: note = Description of Artificial Sequence: Synthetic oligonucleotide
  source 1..19: mol_type = other RNA
acgacttccagcgtttcct

SEQ ID NO: 192 - RNA, 19 nt, synthetic construct
  misc_feature 1..19: note = Description of Artificial Sequence: Synthetic oligonucleotide
  source 1..19: mol_type = other RNA
gtcgtttggcttgtggtgt

SEQ ID NO: 193 - RNA, 19 nt, synthetic construct
  misc_feature 1..19: note = Description of Artificial Sequence: Synthetic oligonucleotide
  source 1..19: mol_type = other RNA
acaccacaagccaaacgac

SEQ ID NO: 194 - RNA, 19 nt, synthetic construct
  misc_feature 1..19: note = Description of Artificial Sequence: Synthetic oligonucleotide
  source 1..19: mol_type = other RNA
tcgtttggcttgtggtgta

SEQ ID NO: 195 - RNA, 19 nt, synthetic construct
  misc_feature 1..19: note = Description of Artificial Sequence: Synthetic oligonucleotide
  source 1..19: mol_type = other RNA
tacaccacaagccaaacga

SEQ ID NO: 196 - RNA, 19 nt, synthetic construct
  misc_feature 1..19: note = Description of Artificial Sequence: Synthetic oligonucleotide
  source 1..19: mol_type = other RNA
cgtttggcttgtggtgtaa

SEQ ID NO: 197 - RNA, 19 nt, synthetic construct
  misc_feature 1..19: note = Description of Artificial Sequence: Synthetic oligonucleotide
  source 1..19: mol_type = other RNA
ttacaccacaagccaaacg

SEQ ID NO: 198 - RNA, 19 nt, synthetic construct
  misc_feature 1..19: note = Description of Artificial Sequence: Synthetic oligonucleotide
  source 1..19: mol_type = other RNA
gtttggcttgtggtgtaat

SEQ ID NO: 199 - RNA, 19 nt, synthetic construct
  misc_feature 1..19: note = Description of Artificial Sequence: Synthetic oligonucleotide
  source 1..19: mol_type = other RNA
attacaccacaagccaaac

SEQ ID NO: 200 - RNA, 19 nt, synthetic construct
  misc_feature 1..19: note = Description of Artificial Sequence: Synthetic oligonucleotide
  source 1..19: mol_type = other RNA
ttggcttgtggtgtaattg

SEQ ID NO: 201 - RNA, 19 nt, synthetic construct
  misc_feature 1..19: note = Description of Artificial Sequence: Synthetic oligonucleotide
  source 1..19: mol_type = other RNA
caattacaccacaagccaa

SEQ ID NO: 202 - RNA, 19 nt, synthetic construct
  misc_feature 1..19: note = Description of Artificial Sequence: Synthetic oligonucleotide
  source 1..19: mol_type = other RNA
ggcttgtggtgtaattggg

SEQ ID NO: 203 - RNA, 19 nt, synthetic construct
  misc_feature 1..19: note = Description of Artificial Sequence: Synthetic oligonucleotide
  source 1..19: mol_type = other RNA
cccaattacaccacaagcc

SEQ ID NO: 204 - RNA, 19 nt, synthetic construct
  misc_feature 1..19: note = Description of Artificial Sequence: Synthetic oligonucleotide
  source 1..19: mol_type = other RNA
gcttgtggtgtaattggga

SEQ ID NO: 205 - RNA, 19 nt, synthetic construct
  misc_feature 1..19: note = Description of Artificial Sequence: Synthetic oligonucleotide
  source 1..19: mol_type = other RNA
tcccaattacaccacaagc

SEQ ID NO: 206 - RNA, 19 nt, synthetic construct
  misc_feature 1..19: note = Description of Artificial Sequence: Synthetic oligonucleotide
  source 1..19: mol_type = other RNA
cttgtggtgtaattgggat

SEQ ID NO: 207 - RNA, 19 nt, synthetic construct
  misc_feature 1..19: note = Description of Artificial Sequence: Synthetic oligonucleotide
  source 1..19: mol_type = other RNA
atcccaattacaccacaag

SEQ ID NO: 208 - RNA, 19 nt, synthetic construct
  misc_feature 1..19: note = Description of Artificial Sequence: Synthetic oligonucleotide
  source 1..19: mol_type = other RNA
tgtggtgtaattgggatcg

SEQ ID NO: 209 - RNA, 19 nt, synthetic construct
  misc_feature 1..19: note = Description of Artificial Sequence: Synthetic oligonucleotide
  source 1..19: mol_type = other RNA
cgatcccaattacaccaca

SEQ ID NO: 210 - RNA, 19 nt, synthetic construct
  misc_feature 1..19: note = Description of Artificial Sequence: Synthetic oligonucleotide
  source 1..19: mol_type = other RNA
gtggtgtaattgggatcgc

SEQ ID NO: 211 - RNA, 19 nt, synthetic construct
  misc_feature 1..19: note = Description of Artificial Sequence: Synthetic oligonucleotide
  source 1..19: mol_type = other RNA
gcgatcccaattacaccac

SEQ ID NO: 212 - RNA, 19 nt, synthetic construct
  misc_feature 1..19: note = Description of Artificial Sequence: Synthetic oligonucleotide
  source 1..19: mol_type = other RNA
tggtgtaattgggatcgcc

SEQ ID NO: 213 - RNA, 19 nt, synthetic construct
  misc_feature 1..19: note = Description of Artificial Sequence: Synthetic oligonucleotide
  source 1..19: mol_type = other RNA
ggcgatcccaattacacca

SEQ ID NO: 214 - RNA, 19 nt, synthetic construct
  misc_feature 1..19: note = Description of Artificial Sequence: Synthetic oligonucleotide
  source 1..19: mol_type = other RNA
gtaattgggatcgcccaat

SEQ ID NO: 215 - RNA, 19 nt, synthetic construct
  misc_feature 1..19: note = Description of Artificial Sequence: Synthetic oligonucleotide
  source 1..19: mol_type = other RNA
attgggcgatcccaattac

SEQ ID NO: 216 - RNA, 19 nt, synthetic construct
  misc_feature 1..19: note = Description of Artificial Sequence: Synthetic oligonucleotide
  source 1..19: mol_type = other RNA
taattgggatcgcccaata

SEQ ID NO: 217 - RNA, 19 nt, synthetic construct
  misc_feature 1..19: note = Description of Artificial Sequence: Synthetic oligonucleotide
  source 1..19: mol_type = other RNA
tattgggcgatcccaatta

SEQ ID NO: 218 - RNA, 19 nt, synthetic construct
  misc_feature 1..19: note = Description of Artificial Sequence: Synthetic oligonucleotide
  source 1..19: mol_type = other RNA
aattgggatcgcccaataa

SEQ ID NO: 219 - RNA, 19 nt, synthetic construct
  misc_feature 1..19: note = Description of Artificial Sequence: Synthetic oligonucleotide
  source 1..19: mol_type = other RNA
ttattgggcgatcccaatt

SEQ ID NO: 220 - RNA, 19 nt, synthetic construct
  misc_feature 1..19: note = Description of Artificial Sequence: Synthetic oligonucleotide
  source 1..19: mol_type = other RNA
attgggatcgcccaataaa

SEQ ID NO: 221 - RNA, 19 nt, synthetic construct
  misc_feature 1..19: note = Description of Artificial Sequence: Synthetic oligonucleotide
  source 1..19: mol_type = other RNA
tttattgggcgatcccaat

SEQ ID NO: 222 - RNA, 19 nt, synthetic construct
  misc_feature 1..19: note = Description of Artificial Sequence: Synthetic oligonucleotide
  source 1..19: mol_type = other RNA
ttgggatcgcccaataaac

SEQ ID NO: 223 - RNA, 19 nt, synthetic construct
  misc_feature 1..19: note = Description of Artificial Sequence: Synthetic oligonucleotide
  source 1..19: mol_type = other RNA
gtttattgggcgatcccaa

SEQ ID NO: 224 - RNA, 19 nt, synthetic construct
  misc_feature 1..19: note = Description of Artificial Sequence: Synthetic oligonucleotide
  source 1..19: mol_type = other RNA
tgggatcgcccaataaaca

SEQ ID NO: 225 - RNA, 19 nt, synthetic construct
  misc_feature 1..19: note = Description of Artificial Sequence: Synthetic oligonucleotide
  source 1..19: mol_type = other RNA
tgtttattgggcgatccca

SEQ ID NO: 226 - RNA, 19 nt, synthetic construct
  misc_feature 1..19: note = Description of Artificial Sequence: Synthetic oligonucleotide
  source 1..19: mol_type = other RNA
gggatcgcccaataaacat

SEQ ID NO: 227 - RNA, 19 nt, synthetic construct
  misc_feature 1..19: note = Description of Artificial Sequence: Synthetic oligonucleotide
  source 1..19: mol_type = other RNA
atgtttattgggcgatccc

SEQ ID NO: 228 - RNA, 19 nt, synthetic construct
  misc_feature 1..19: note = Description of Artificial Sequence: Synthetic oligonucleotide
  source 1..19: mol_type = other RNA
atcgcccaataaacattcc

SEQ ID NO: 229 - RNA, 19 nt, synthetic construct
  misc_feature 1..19: note = Description of Artificial Sequence: Synthetic oligonucleotide
  source 1..19: mol_type = other RNA
ggaatgtttattgggcgat

SEQ ID NO: 230 - RNA, 19 nt, synthetic construct
  misc_feature 1..19: note = Description of Artificial Sequence: Synthetic oligonucleotide
  source 1..19: mol_type = other RNA
ccaataaacattcccttgg

SEQ ID NO: 231 - RNA, 19 nt, synthetic construct
  misc_feature 1..19: note = Description of Artificial Sequence: Synthetic oligonucleotide
  source 1..19: mol_type = other RNA
ccaagggaatgtttattgg

SEQ ID NO: 232 - RNA, 19 nt, synthetic construct
  misc_feature 1..19: note = Description of Artificial Sequence: Synthetic oligonucleotide
  source 1..19: mol_type = other RNA
caataaacattcccttgga

SEQ ID NO: 233 - RNA, 19 nt, synthetic construct
  misc_feature 1..19: note = Description of Artificial Sequence: Synthetic oligonucleotide
  source 1..19: mol_type = other RNA
tccaagggaatgtttattg

SEQ ID NO: 234 - RNA, 19 nt, synthetic construct
  misc_feature 1..19: note = Description of Artificial Sequence: Synthetic oligonucleotide
  source 1..19: mol_type = other RNA
aataaacattcccttggat

SEQ ID NO: 235 - RNA, 19 nt, synthetic construct
  misc_feature 1..19: note = Description of Artificial Sequence: Synthetic oligonucleotide
  source 1..19: mol_type = other RNA
atccaagggaatgtttatt

SEQ ID NO: 236 - RNA, 19 nt, synthetic construct
  misc_feature 1..19: note = Description of Artificial Sequence: Synthetic oligonucleotide
  source 1..19: mol_type = other RNA
ataaacattcccttggatg

SEQ ID NO: 237 - RNA, 19 nt, synthetic construct
  misc_feature 1..19: note = Description of Artificial Sequence: Synthetic oligonucleotide
  source 1..19: mol_type = other RNA
catccaagggaatgtttat

SEQ ID NO: 238 - RNA, 19 nt, synthetic construct
  misc_feature 1..19: note = Description of Artificial Sequence: Synthetic oligonucleotide
  source 1..19: mol_type = other RNA
taaacattcccttggatgt

SEQ ID NO: 239 - RNA, 19 nt, synthetic construct
  misc_feature 1..19: note = Description of Artificial Sequence: Synthetic oligonucleotide
  source 1..19: mol_type = other RNA
acatccaagggaatgttta

SEQ ID NO: 240 - RNA, 19 nt, synthetic construct
  misc_feature 1..19: note = Description of Artificial Sequence: Synthetic oligonucleotide
  source 1..19: mol_type = other RNA
aaacattcccttggatgta

SEQ ID NO: 241 - RNA, 19 nt, synthetic construct
  misc_feature 1..19: note = Description of Artificial Sequence: Synthetic oligonucleotide
  source 1..19: mol_type = other RNA
tacatccaagggaatgttt

SEQ ID NO: 242 - RNA, 19 nt, synthetic construct
  misc_feature 1..19: note = Description of Artificial Sequence: Synthetic oligonucleotide
  source 1..19: mol_type = other RNA
aacattcccttggatgtag

SEQ ID NO: 243 - RNA, 19 nt, synthetic construct
  misc_feature 1..19: note = Description of Artificial Sequence: Synthetic oligonucleotide
  source 1..19: mol_type = other RNA
ctacatccaagggaatgtt

SEQ ID NO: 244 - RNA, 19 nt, synthetic construct
  misc_feature 1..19: note = Description of Artificial Sequence: Synthetic oligonucleotide
  source 1..19: mol_type = other RNA
attcccttggatgtagtct

SEQ ID NO: 245 - RNA, 19 nt, synthetic construct
  misc_feature 1..19: note = Description of Artificial Sequence: Synthetic oligonucleotide
  source 1..19: mol_type = other RNA
agactacatccaagggaat

SEQ ID NO: 246 - RNA, 19 nt, synthetic construct
  misc_feature 1..19: note = Description of Artificial Sequence: Synthetic oligonucleotide
  source 1..19: mol_type = other RNA
cttggatgtagtctgaggc

SEQ ID NO: 247 - RNA, 19 nt, synthetic construct
  misc_feature 1..19: note = Description of Artificial Sequence: Synthetic oligonucleotide
  source 1..19: mol_type = other RNA
gcctcagactacatccaag

SEQ ID NO: 248 - RNA, 19 nt, synthetic construct
  misc_feature 1..19: note = Description of Artificial Sequence: Synthetic oligonucleotide
  source 1..19: mol_type = other RNA
ctgaggccccttaactcat

SEQ ID NO: 249 - RNA, 19 nt, synthetic construct
  misc_feature 1..19: note = Description of Artificial Sequence: Synthetic oligonucleotide
  source 1..19: mol_type = other RNA
atgagttaaggggcctcag

SEQ ID NO: 250 - RNA, 19 nt, synthetic construct
  misc_feature 1..19: note = Description of Artificial Sequence: Synthetic oligonucleotide
  source 1..19: mol_type = other RNA
gaggccccttaactcatct

SEQ ID NO: 251 - RNA, 19 nt, synthetic construct
  misc_feature 1..19: note = Description of Artificial Sequence: Synthetic oligonucleotide
  source 1..19: mol_type = other RNA
agatgagttaaggggcctc

SEQ ID NO: 252 - RNA, 19 nt, synthetic construct
  misc_feature 1..19: note = Description of Artificial Sequence: Synthetic oligonucleotide
  source 1..19: mol_type = other RNA
aggccccttaactcatctg

SEQ ID NO: 253 - RNA, 19 nt, synthetic construct
  misc_feature 1..19: note = Description of Artificial Sequence: Synthetic oligonucleotide
  source 1..19: mol_type = other RNA
cagatgagttaaggggcct

SEQ ID NO: 254 - RNA, 19 nt, synthetic construct
  misc_feature 1..19: note = Description of Artificial Sequence: Synthetic oligonucleotide
  source 1..19: mol_type = other RNA
ccccttaactcatctgtta

SEQ ID NO: 255 - RNA, 19 nt, synthetic construct
  misc_feature 1..19: note = Description of Artificial Sequence: Synthetic oligonucleotide
  source 1..19: mol_type = other RNA
taacagatgagttaagggg

SEQ ID NO: 256 - RNA, 19 nt, synthetic construct
  misc_feature 1..19: note = Description of Artificial Sequence: Synthetic oligonucleotide
  source 1..19: mol_type = other RNA
cccttaactcatctgttat

SEQ ID NO: 257 - RNA, 19 nt, synthetic construct
  misc_feature 1..19: note = Description of Artificial Sequence: Synthetic oligonucleotide
  source 1..19: mol_type = other RNA
ataacagatgagttaaggg

SEQ ID NO: 258 - RNA, 19 nt, synthetic construct
  misc_feature 1..19: note = Description of Artificial Sequence: Synthetic oligonucleotide
  source 1..19: mol_type = other RNA
ccttaactcatctgttatc

SEQ ID NO: 259 - RNA, 19 nt, synthetic construct
  misc_feature 1..19: note = Description of Artificial Sequence: Synthetic oligonucleotide
  source 1..19: mol_type = other RNA
gataacagatgagttaagg

SEQ ID NO: 260 - RNA, 19 nt, synthetic construct
  misc_feature 1..19: note = Description of Artificial Sequence: Synthetic oligonucleotide
  source 1..19: mol_type = other RNA
cttaactcatctgttatcc

SEQ ID NO: 261 - RNA, 19 nt, synthetic construct
  misc_feature 1..19: note = Description of Artificial Sequence: Synthetic oligonucleotide
  source 1..19: mol_type = other RNA
ggataacagatgagttaag

SEQ ID NO: 262 - RNA, 19 nt, synthetic construct
  misc_feature 1..19: note = Description of Artificial Sequence: Synthetic oligonucleotide
  source 1..19: mol_type = other RNA
ttaactcatctgttatcct

SEQ ID NO: 263 - RNA, 19 nt, synthetic construct
  misc_feature 1..19: note = Description of Artificial Sequence: Synthetic oligonucleotide
  source 1..19: mol_type = other RNA
aggataacagatgagttaa

SEQ ID NO: 264 - RNA, 19 nt, synthetic construct
  misc_feature 1..19: note = Description of Artificial Sequence: Synthetic oligonucleotide
  source 1..19: mol_type = other RNA
taactcatctgttatcctg

SEQ ID NO: 265 - RNA, 19 nt, synthetic construct
  misc_feature 1..19: note = Description of Artificial Sequence: Synthetic oligonucleotide
  source 1..19: mol_type = other RNA
caggataacagatgagtta

SEQ ID NO: 266 - RNA, 19 nt, synthetic construct
  misc_feature 1..19: note = Description of Artificial Sequence: Synthetic oligonucleotide
  source 1..19: mol_type = other RNA
aactcatctgttatcctgc

SEQ ID NO: 267 - RNA, 19 nt, synthetic construct
  misc_feature 1..19: note = Description of Artificial Sequence: Synthetic oligonucleotide
  source 1..19: mol_type = other RNA
gcaggataacagatgagtt

SEQ ID NO: 268 - RNA, 19 nt, synthetic construct
  misc_feature 1..19: note = Description of Artificial Sequence: Synthetic oligonucleotide
  source 1..19: mol_type = other RNA
gttatcctgctagctgtag

SEQ ID NO: 269 - RNA, 19 nt, synthetic construct
  misc_feature 1..19: note = Description of Artificial Sequence: Synthetic oligonucleotide
  source 1..19: mol_type = other RNA
ctacagctagcaggataac

SEQ ID NO: 270 - RNA, 19 nt, synthetic construct
  misc_feature 1..19: note = Description of Artificial Sequence: Synthetic oligonucleotide
  source 1..19: mol_type = other RNA
ctgctagctgtagaaatgt

SEQ ID NO: 271 - RNA, 19 nt, synthetic construct
  misc_feature 1..19: note = Description of Artificial Sequence: Synthetic oligonucleotide
  source 1..19: mol_type = other RNA
acatttctacagctagcag

SEQ ID NO: 272 - RNA, 19 nt, synthetic construct
  misc_feature 1..19: note = Description of Artificial Sequence: Synthetic oligonucleotide
  source 1..19: mol_type = other RNA
tgctagctgtagaaatgta

SEQ ID NO: 273 - RNA, 19 nt, synthetic construct
  misc_feature 1..19: note = Description of Artificial Sequence: Synthetic oligonucleotide
  source 1..19: mol_type = other RNA
tacatttctacagctagca

SEQ ID NO: 274 - RNA, 19 nt, synthetic construct
  misc_feature 1..19: note = Description of Artificial Sequence: Synthetic oligonucleotide
  source 1..19: mol_type = other RNA
gctgtagaaatgtatcctg

SEQ ID NO: 275 - RNA, 19 nt, synthetic construct
  misc_feature 1..19: note = Description of Artificial Sequence: Synthetic oligonucleotide
  source 1..19: mol_type = other RNA
caggatacatttctacagc

SEQ ID NO: 276 - RNA, 19 nt, synthetic construct
  misc_feature 1..19: note = Description of Artificial Sequence: Synthetic oligonucleotide
  source 1..19: mol_type = other RNA
ctgtagaaatgtatcctga

SEQ ID NO: 277 - RNA, 19 nt, synthetic construct
  misc_feature 1..19: note = Description of Artificial Sequence: Synthetic oligonucleotide
  source 1..19: mol_type = other RNA
tcaggatacatttctacag

SEQ ID NO: 278 - RNA, 19 nt, synthetic construct
  misc_feature 1..19: note = Description of Artificial Sequence: Synthetic oligonucleotide
  source 1..19: mol_type = other RNA
tgtagaaatgtatcctgat

SEQ ID NO: 279 - RNA, 19 nt, synthetic construct
  misc_feature 1..19: note = Description of Artificial Sequence: Synthetic oligonucleotide
  source 1..19: mol_type = other RNA
atcaggatacatttctaca

SEQ ID NO: 280 - RNA, 19 nt, synthetic construct
  misc_feature 1..19: note = Description of Artificial Sequence: Synthetic oligonucleotide
  source 1..19: mol_type = other RNA
gtagaaatgtatcctgata

SEQ ID NO: 281 - RNA, 19 nt, synthetic construct
  misc_feature 1..19: note = Description of Artificial Sequence: Synthetic oligonucleotide
  source 1..19: mol_type = other RNA
tatcaggatacatttctac

SEQ ID NO: 282 - RNA, 19 nt, synthetic construct
  misc_feature 1..19: note = Description of Artificial Sequence: Synthetic oligonucleotide
  source 1..19: mol_type = other RNA
aaatgtatcctgataaaca

SEQ ID NO: 283 - RNA, 19 nt, synthetic construct
  misc_feature 1..19: note = Description of Artificial Sequence: Synthetic oligonucleotide
  source 1..19: mol_type = other RNA
tgtttatcaggatacattt

SEQ ID NO: 284 - RNA, 19 nt, synthetic construct
  misc_feature 1..19: note = Description of Artificial Sequence: Synthetic oligonucleotide
  source 1..19: mol_type = other RNA
gtatcctgataaacattaa

SEQ ID NO: 285 - RNA, 19 nt, synthetic construct
  misc_feature 1..19: note = Description of Artificial Sequence: Synthetic oligonucleotide
  source 1..19: mol_type = other RNA
ttaatgtttatcaggatac

SEQ ID NO: 286 - RNA, 19 nt, synthetic construct
  misc_feature 1..19: note = Description of Artificial Sequence: Synthetic oligonucleotide
  source 1..19: mol_type = other RNA
ttaaacactgtaatcttaa

SEQ ID NO: 287 - RNA, 19 nt, synthetic construct
  misc_feature 1..19: note = Description of Artificial Sequence: Synthetic oligonucleotide
  source 1..19: mol_type = other RNA
ttaagattacagtgtttaa

SEQ ID NO: 288 - RNA, 19 nt, synthetic construct
  misc_feature 1..19: note = Description of Artificial Sequence: Synthetic oligonucleotide
  source 1..19: mol_type = other RNA
actgtaatcttaaaagtgt

SEQ ID NO: 289 - RNA, 19 nt, synthetic construct
  misc_feature 1..19: note = Description of Artificial Sequence: Synthetic oligonucleotide
  source 1..19: mol_type = other RNA
acacttttaagattacagt

SEQ ID NO: 290 - RNA, 19 nt, synthetic construct
  misc_feature 1..19: note = Description of Artificial Sequence: Synthetic oligonucleotide
  source 1..19: mol_type = other RNA
ctgtaatcttaaaagtgta

SEQ ID NO: 291 - RNA, 19 nt, synthetic construct
  misc_feature 1..19: note = Description of Artificial Sequence: Synthetic oligonucleotide
  source 1..19: mol_type = other RNA
tacacttttaagattacag

SEQ ID NO: 292 - RNA, 19 nt, synthetic construct
  misc_feature 1..19: note = Description of Artificial Sequence: Synthetic oligonucleotide
  source 1..19: mol_type = other RNA
tgtaatcttaaaagtgtaa

SEQ ID NO: 293 - RNA, 19 nt, synthetic construct
  misc_feature 1..19: note = Description of Artificial Sequence: Synthetic oligonucleotide
  source 1..19: mol_type = other RNA
ttacacttttaagattaca

SEQ ID NO: 294 - RNA, 19 nt, synthetic construct
  misc_feature 1..19: note = Description of Artificial Sequence: Synthetic oligonucleotide
  source 1..19: mol_type = other RNA
gtaatcttaaaagtgtaat

SEQ ID NO: 295 - RNA, 19 nt, synthetic construct
  misc_feature 1..19: note = Description of Artificial Sequence: Synthetic oligonucleotide
  source 1..19: mol_type = other RNA
attacacttttaagattac

SEQ ID NO: 296 - RNA, 19 nt, synthetic construct
  misc_feature 1..19: note = Description of Artificial Sequence: Synthetic oligonucleotide
  source 1..19: mol_type = other RNA
cttaaaagtgtaattgtgt

SEQ ID NO: 297 - RNA, 19 nt, synthetic construct
  misc_feature 1..19: note = Description of Artificial Sequence: Synthetic oligonucleotide
  source 1..19: mol_type = other RNA
acacaattacacttttaag

SEQ ID NO: 298 - RNA, 19 nt, synthetic construct
  misc_feature 1..19: note = Description of Artificial Sequence: Synthetic oligonucleotide
  source 1..19: mol_type = other RNA
tacctgtagtgagaaactg

SEQ ID NO: 299 - RNA, 19 nt, synthetic construct
  misc_feature 1..19: note = Description of Artificial Sequence: Synthetic oligonucleotide
  source 1..19: mol_type = other RNA
cagtttctcactacaggta

SEQ ID NO: 300 - RNA, 19 nt, synthetic construct
  misc_feature 1..19: note = Description of Artificial Sequence: Synthetic oligonucleotide
  source 1..19: mol_type = other RNA
ttatgatcacttggaagat

SEQ ID NO: 301 - RNA, 19 nt, synthetic construct
  misc_feature 1..19: note = Description of Artificial Sequence: Synthetic oligonucleotide
  source 1..19: mol_type = other RNA
atcttccaagtgatcataa

SEQ ID NO: 302 - RNA, 19 nt, synthetic construct
  misc_feature 1..19: note = Description of Artificial Sequence: Synthetic oligonucleotide
  source 1..19: mol_type = other RNA
atgatcacttggaagattt

SEQ ID NO: 303 - RNA, 19 nt, synthetic construct
  misc_feature 1..19: note = Description of Artificial Sequence: Synthetic oligonucleotide
  source 1..19: mol_type = other RNA
aaatcttccaagtgatcat

SEQ ID NO: 304 - RNA, 19 nt, synthetic construct
  misc_feature 1..19: note = Description of Artificial Sequence: Synthetic oligonucleotide
  source 1..19: mol_type = other RNA
atcacttggaagatttgta

SEQ ID NO: 305 - RNA, 19 nt, synthetic construct
  misc_feature 1..19: note = Description of Artificial Sequence: Synthetic oligonucleotide
  source 1..19: mol_type = other RNA
tacaaatcttccaagtgat

SEQ ID NO: 306 - RNA, 19 nt, synthetic construct
  misc_feature 1..19: note = Description of Artificial Sequence: Synthetic oligonucleotide
  source 1..19: mol_type = other RNA
tggaagatttgtatagttt

SEQ ID NO: 307 - RNA, 19 nt, synthetic construct
  misc_feature 1..19: note = Description of Artificial Sequence: Synthetic oligonucleotide
  source 1..19: mol_type = other RNA
aaactatacaaatcttcca

SEQ ID NO: 308 - RNA, 19 nt, synthetic construct
  misc_feature 1..19: note = Description of Artificial Sequence: Synthetic oligonucleotide
  source 1..19: mol_type = other RNA
tataaaactcagttaaaat

SEQ ID NO: 309 - RNA, 19 nt, synthetic construct
  misc_feature 1..19: note = Description of Artificial Sequence: Synthetic oligonucleotide
  source 1..19: mol_type = other RNA
attttaactgagttttata

SEQ ID NO: 310 - RNA, 19 nt, synthetic construct
  misc_feature 1..19: note = Description of Artificial Sequence: Synthetic oligonucleotide
  source 1..19: mol_type = other RNA
aaactcagttaaaatgtct

SEQ ID NO: 311 - RNA, 19 nt, synthetic construct
  misc_feature 1..19: note = Description of Artificial Sequence: Synthetic oligonucleotide
  source 1..19: mol_type = other RNA
agacattttaactgagttt

SEQ ID NO: 312 - RNA, 19 nt, synthetic construct
  misc_feature 1..19: note = Description of Artificial Sequence: Synthetic oligonucleotide
  source 1..19: mol_type = other RNA
gtctgtttcaatgacctgt

SEQ ID NO: 313 - RNA, 19 nt, synthetic construct
  misc_feature 1..19: note = Description of Artificial Sequence: Synthetic oligonucleotide
  source 1..19: mol_type = other RNA
acaggtcattgaaacagac

SEQ ID NO: 314 - RNA, 19 nt, synthetic construct
  misc_feature 1..19: note = Description of Artificial Sequence: Synthetic oligonucleotide
  source 1..19: mol_type = other RNA
atgacctgtattttgccag

SEQ ID NO: 315 - RNA, 19 nt, synthetic construct
  misc_feature 1..19: note = Description of Artificial Sequence: Synthetic oligonucleotide
  source 1..19: mol_type = other RNA
ctggcaaaatacaggtcat

SEQ ID NO: 316 - RNA, 19 nt, synthetic construct
  misc_feature 1..19: note = Description of Artificial Sequence: Synthetic oligonucleotide
  source 1..19: mol_type = other RNA
acctgtattttgccagact

SEQ ID NO: 317 - RNA, 19 nt, synthetic construct
  misc_feature 1..19: note = Description of Artificial Sequence: Synthetic oligonucleotide
  source 1..19: mol_type = other RNA
agtctggcaaaatacaggt

SEQ ID NO: 318 - RNA, 19 nt, synthetic construct
  misc_feature 1..19: note = Description of Artificial Sequence: Synthetic oligonucleotide
  source 1..19: mol_type = other RNA
cctgtattttgccagactt

SEQ ID NO: 319 - RNA, 19 nt, synthetic construct
  misc_feature 1..19: note = Description of Artificial Sequence: Synthetic oligonucleotide
  source 1..19: mol_type = other RNA
aagtctggcaaaatacagg

SEQ ID NO: 320 - RNA, 19 nt, synthetic construct
  misc_feature 1..19: note = Description of Artificial Sequence: Synthetic oligonucleotide
  source 1..19: mol_type = other RNA
taaatcacagatgggtatt

SEQ ID NO: 321 - RNA, 19 nt, synthetic construct
  misc_feature 1..19: note = Description of Artificial Sequence: Synthetic oligonucleotide
  source 1..19: mol_type = other RNA
aatacccatctgtgattta

SEQ ID NO: 322 - RNA, 19 nt, synthetic construct
  misc_feature 1..19: note = Description of Artificial Sequence: Synthetic oligonucleotide
  source 1..19: mol_type = other RNA
atcacagatgggtattaaa

SEQ ID NO: 323 - RNA, 19 nt, synthetic construct
  misc_feature 1..19: note = Description of Artificial Sequence: Synthetic oligonucleotide
  source 1..19: mol_type = other RNA
tttaatacccatctgtgat

SEQ ID NO: 324 - RNA, 19 nt, synthetic construct
  misc_feature 1..19: note = Description of Artificial Sequence: Synthetic oligonucleotide
  source 1..19: mol_type = other RNA
tcacagatgggtattaaac

SEQ ID NO: 325 - RNA, 19 nt, synthetic construct
  misc_feature 1..19: note = Description of Artificial Sequence: Synthetic oligonucleotide
  source 1..19: mol_type = other RNA
gtttaatacccatctgtga

SEQ ID NO: 326 - RNA, 19 nt, synthetic construct
  misc_feature 1..19: note = Description of Artificial Sequence: Synthetic oligonucleotide
  source 1..19: mol_type = other RNA
acagatgggtattaaactt

SEQ ID NO: 327 - RNA, 19 nt, synthetic construct
  misc_feature 1..19: note = Description of Artificial Sequence: Synthetic oligonucleotide
  source 1..19: mol_type = other RNA
aagtttaatacccatctgt

SEQ ID NO: 328 - RNA, 19 nt, synthetic construct
  misc_feature 1..19: note = Description of Artificial Sequence: Synthetic oligonucleotide
  source 1..19: mol_type = other RNA
cagatgggtattaaacttg

SEQ ID NO: 329 - RNA, 19 nt, synthetic construct
  misc_feature 1..19: note = Description of Artificial Sequence: Synthetic oligonucleotide
  source 1..19: mol_type = other RNA
caagtttaatacccatctg

SEQ ID NO: 330 - RNA, 19 nt, synthetic construct
  misc_feature 1..19: note = Description of Artificial Sequence: Synthetic oligonucleotide
  source 1..19: mol_type = other RNA
agatgggtattaaacttgt

SEQ ID NO: 331 - RNA, 19 nt, synthetic construct
  misc_feature 1..19: note = Description of Artificial Sequence: Synthetic oligonucleotide
  source 1..19: mol_type = other RNA
acaagtttaatacccatct

SEQ ID NO: 332 - RNA, 19 nt, synthetic construct
  misc_feature 1..19: note = Description of Artificial Sequence: Synthetic oligonucleotide
  source 1..19: mol_type = other RNA
atgggtattaaacttgtca

SEQ ID NO: 333 - RNA, 19 nt, synthetic construct
  misc_feature 1..19: note = Description of Artificial Sequence: Synthetic oligonucleotide
  source 1..19: mol_type = other RNA
tgacaagtttaatacccat

SEQ ID NO: 334 - RNA, 19 nt, synthetic construct
  misc_feature 1..19: note = Description of Artificial Sequence: Synthetic oligonucleotide
  source 1..19: mol_type = other RNA
taaacttgtcagaatttct

SEQ ID NO: 335 - RNA, 19 nt, synthetic construct
  misc_feature 1..19: note = Description of Artificial Sequence: Synthetic oligonucleotide
  source 1..19: mol_type = other RNA
agaaattctgacaagttta

SEQ ID NO: 336 - RNA, 19 nt, synthetic construct
  misc_feature 1..19: note = Description of Artificial Sequence: Synthetic oligonucleotide
  source 1..19: mol_type = other RNA
tcattcaagcctgtgaata

SEQ ID NO: 337 - RNA, 19 nt, synthetic construct
  misc_feature 1..19: note = Description of Artificial Sequence: Synthetic oligonucleotide
  source 1..19: mol_type = other RNA
tattcacaggcttgaatga

SEQ ID NO: 338 - RNA, 19 nt, synthetic construct
  misc_feature 1..19: note = Description of Artificial Sequence: Synthetic oligonucleotide
  source 1..19: mol_type = other RNA
cattcaagcctgtgaataa

SEQ ID NO: 339 - RNA, 19 nt, synthetic construct
  misc_feature 1..19: note = Description of Artificial Sequence: Synthetic oligonucleotide
  source 1..19: mol_type = other RNA
ttattcacaggcttgaatg

SEQ ID NO: 340 - RNA, 19 nt, synthetic construct
  misc_feature 1..19: note = Description of Artificial Sequence: Synthetic oligonucleotide
  source 1..19: mol_type = other RNA
aataaaaaccctgtatggc

SEQ ID NO: 341 - RNA, 19 nt, synthetic construct
  misc_feature 1..19: note = Description of Artificial Sequence: Synthetic oligonucleotide
  source 1..19: mol_type = other RNA
gccatacagggtttttatt

SEQ ID NO: 342 - RNA, 19 nt, synthetic construct
  misc_feature 1..19: note = Description of Artificial Sequence: Synthetic oligonucleotide
  source 1..19: mol_type = other RNA
ataaaaaccctgtatggca

SEQ ID NO: 343 - RNA, 19 nt, synthetic construct
  misc_feature 1..19: note = Description of Artificial Sequence: Synthetic oligonucleotide
  source 1..19: mol_type = other RNA
tgccatacagggtttttat

SEQ ID NO: 344 - RNA, 19 nt, synthetic construct
  misc_feature 1..19: note = Description of Artificial Sequence: Synthetic oligonucleotide
  source 1..19: mol_type = other RNA
aaccctgtatggcacttat

SEQ ID NO: 345 - RNA, 19 nt, synthetic construct
  misc_feature 1..19: note = Description of Artificial Sequence: Synthetic oligonucleotide
  source 1..19: mol_type = other RNA
ataagtgccatacagggtt

SEQ ID NO: 346 - RNA, 19 nt, synthetic construct
  misc_feature 1..19: note = Description of Artificial Sequence: Synthetic oligonucleotide
  source 1..19: mol_type = other RNA
accctgtatggcacttatt

SEQ ID NO: 347 - RNA, 19 nt, synthetic construct
  misc_feature 1..19: note = Description of Artificial Sequence: Synthetic oligonucleotide
  source 1..19: mol_type = other RNA
aataagtgccatacagggt

SEQ ID NO: 348 - RNA, 19 nt, synthetic construct
  misc_feature 1..19: note = Description of Artificial Sequence: Synthetic oligonucleotide
  source 1..19: mol_type = other RNA
gaggctattaaaagaatcc

SEQ ID NO: 349 - RNA, 19 nt, synthetic construct
  misc_feature 1..19: note = Description of Artificial Sequence: Synthetic oligonucleotide
  source 1..19: mol_type = other RNA
ggattcttttaatagcctc

SEQ ID NO: 350 - RNA, 19 nt, synthetic construct
  misc_feature 1..19: note = Description of Artificial Sequence: Synthetic oligonucleotide
  source 1..19: mol_type = other RNA
aaagaatccaaattcaaac

SEQ ID NO: 351 - RNA, 19 nt, synthetic construct
  misc_feature 1..19: note = Description of Artificial Sequence: Synthetic oligonucleotide
  source 1..19: mol_type = other RNA
gtttgaatttggattcttt

SEQ ID NO: 352 - RNA, 19 nt, synthetic construct
  misc_feature 1..19: note = Description of Artificial Sequence: Synthetic oligonucleotide
  source 1..19: mol_type = other RNA
gaatccaaattcaaactaa

SEQ ID NO: 353 - RNA, 19 nt, synthetic construct
  misc_feature 1..19: note = Description of Artificial Sequence: Synthetic oligonucleotide
  source 1..19: mol_type = other RNA
ttagtttgaatttggattc

SEQ ID NO: 354 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cggaggtctggcctataactt

SEQ ID NO: 355 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttataggccagacctccgtt

SEQ ID NO: 356 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggaggtctggcctataaactt

SEQ ID NO: 357 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttataggccagacctcctt

SEQ ID NO: 358 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaggtctggcctataaagctt

SEQ ID NO: 359 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctttataggccagacctctt

SEQ ID NO: 360 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aggtctggcctataaagtctt

SEQ ID NO: 361 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tactttataggccagaccttt

SEQ ID NO: 362 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggtctggcctataaagtactt

SEQ ID NO: 363 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttactttataggccagacctt

SEQ ID NO: 364 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctggcctataaagtagtctt

SEQ ID NO: 365 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tactactttataggccagatt

SEQ ID NO: 366 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctggcctataaagtagtcctt

SEQ ID NO: 367 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgactactttataggccagtt

SEQ ID NO: 368 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggcctataaagtagtcgctt

SEQ ID NO: 369 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgactactttataggccatt

SEQ ID NO: 370 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggcctataaagtagtcgcctt

SEQ ID NO: 371 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcgactactttataggcctt

SEQ ID NO: 372 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcctataaagtagtcgcgctt

SEQ ID NO: 373 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgcgactactttataggctt

SEQ ID NO: 374 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cctataaagtagtcgcggctt

SEQ ID NO: 375 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccgcgactactttataggtt

SEQ ID NO: 376 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtcgtagtctcctgcagcctt

SEQ ID NO: 377 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgctgcaggagactacgactt

SEQ ID NO: 378 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cgtagtctcctgcagcgtctt

SEQ ID NO: 379 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacgctgcaggagactacgtt

SEQ ID NO: 380 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtagtctcctgcagcgtcctt

SEQ ID NO: 381 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgacgctgcaggagactactt

SEQ ID NO: 382 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagtctcctgcagcgtctctt

SEQ ID NO: 383 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagacgctgcaggagactatt

SEQ ID NO: 384 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atggcgacgaaggccgtgctt

SEQ ID NO: 385 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacggccttcgtcgccattt

SEQ ID NO: 386 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cgacgaaggccgtgtgcgctt

SEQ ID NO: 387 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgcacacggccttcgtcgtt

SEQ ID NO: 388 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaaggccgtgtgcgtgctctt

SEQ ID NO: 389 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagcacgcacacggccttctt

SEQ ID NO: 390 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggccgtgtgcgtgctgaactt

SEQ ID NO: 391 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttcagcacgcacacggcctt

SEQ ID NO: 392 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agggcgacggcccagtgcctt

SEQ ID NO: 393 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcactgggccgtcgcccttt

SEQ ID NO: 394 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcagggcatcatcaattctt

SEQ ID NO: 395 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taattgatgatgccctgcatt

SEQ ID NO: 396 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcagggcatcatcaatttctt

SEQ ID NO: 397 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taaattgatgatgccctgctt

SEQ ID NO: 398 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agggcatcatcaatttcgctt

SEQ ID NO: 399 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgaaattgatgatgcccttt

SEQ ID NO: 400 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gggcatcatcaatttcgactt

SEQ ID NO: 401 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcgaaattgatgatgccctt

SEQ ID NO: 402 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggcatcatcaatttcgagctt

SEQ ID NO: 403 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctcgaaattgatgatgcctt

SEQ ID NO: 404 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcatcatcaatttcgagcctt

SEQ ID NO: 405 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgctcgaaattgatgatgctt

SEQ ID NO: 406 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
catcatcaatttcgagcactt

SEQ ID NO: 407 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgctcgaaattgatgatgtt

SEQ ID NO: 408 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aatttcgagcagaaggaactt

SEQ ID NO: 409 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttccttctgctcgaaatttt

SEQ ID NO: 410 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcgagcagaaggaaagtctt

SEQ ID NO: 411 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tactttccttctgctcgaatt

SEQ ID NO: 412 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgagcagaaggaaagtactt

SEQ ID NO: 413 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttactttccttctgctcgatt

SEQ ID NO: 414 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaggtgtggggaagcattctt

SEQ ID NO: 415 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taatgcttccccacacctttt

SEQ ID NO: 416 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggtgtggggaagcattaactt

SEQ ID NO: 417 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttaatgcttccccacacctt

SEQ ID NO: 418 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gactgactgaaggcctgcctt

SEQ ID NO: 419 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcaggccttcagtcagtctt

SEQ ID NO: 420 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgactgaaggcctgcatctt

SEQ ID NO: 421 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatgcaggccttcagtcagtt

SEQ ID NO: 422 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgactgaaggcctgcatgctt

SEQ ID NO: 423 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcatgcaggccttcagtcatt

SEQ ID NO: 424 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgaaggcctgcatggattctt

SEQ ID NO: 425 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taatccatgcaggccttcatt

SEQ ID NO: 426 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaaggcctgcatggattcctt

SEQ ID NO: 427 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgaatccatgcaggccttctt

SEQ ID NO: 428 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcatggattccatgttcctt

SEQ ID NO: 429 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgaacatggaatccatgcatt

SEQ ID NO: 430 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
catggattccatgttcatctt

SEQ ID NO: 431 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatgaacatggaatccatgtt

SEQ ID NO: 432 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggattccatgttcatgagctt

SEQ ID NO: 433 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctcatgaacatggaatcctt

SEQ ID NO: 434 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttccatgttcatgagtttctt

SEQ ID NO: 435 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taaactcatgaacatggaatt

SEQ ID NO: 436 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gttcatgagtttggagatctt

SEQ ID NO: 437 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatctccaaactcatgaactt

SEQ ID NO: 438 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcatgagtttggagatactt

SEQ ID NO: 439 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttatctccaaactcatgaatt

SEQ ID NO: 440 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgagtttggagataatacctt

SEQ ID NO: 441 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtattatctccaaactcatt

SEQ ID NO: 442 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gagtttggagataatacactt

SEQ ID NO: 443 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgtattatctccaaactctt

SEQ ID NO: 444 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aggctgtaccagtgcaggctt

SEQ ID NO: 445 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcctgcactggtacagccttt

SEQ ID NO: 446 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggctgtaccagtgcaggtctt

SEQ ID NO: 447 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacctgcactggtacagcctt

SEQ ID NO: 448 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcaggtcctcactttaatctt

SEQ ID NO: 449 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tattaaagtgaggacctgctt

SEQ ID NO: 450 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
caggtcctcactttaatcctt

SEQ ID NO: 451 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgattaaagtgaggacctgtt

SEQ ID NO: 452 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcactttaatcctctatcctt

SEQ ID NO: 453 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgatagaggattaaagtgatt

SEQ ID NO: 454 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctatccagaaaacacggtctt

SEQ ID NO: 455 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taccgtgttttctggatagtt

SEQ ID NO: 456 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatccagaaaacacggtgctt

SEQ ID NO: 457 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaccgtgttttctggatatt

SEQ ID NO: 458 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atccagaaaacacggtggctt

SEQ ID NO: 459 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccaccgtgttttctggattt

SEQ ID NO: 460 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ccagaaaacacggtgggcctt

SEQ ID NO: 461 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcccaccgtgttttctggtt

SEQ ID NO: 462 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaaaacacggtgggccaactt

SEQ ID NO: 463 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttggcccaccgtgttttctt

SEQ ID NO: 464 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaaacacggtgggccaaactt

SEQ ID NO: 465 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttggcccaccgtgtttttt

SEQ ID NO: 466 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cggtgggccaaaggatgactt

SEQ ID NO: 467 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcatcctttggcccaccgtt

SEQ ID NO: 468 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aggatgaagagaggcatgctt

SEQ ID NO: 469 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcatgcctctcttcatccttt

SEQ ID NO: 470 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgaagagaggcatgttgctt

SEQ ID NO: 471 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaacatgcctctcttcattt

SEQ ID NO: 472 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gagaggcatgttggagacctt

SEQ ID NO: 473 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtctccaacatgcctctctt

SEQ ID NO: 474 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agaggcatgttggagactctt

SEQ ID NO: 475 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagtctccaacatgcctcttt

SEQ ID NO: 476 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgttggagacttgggcactt

SEQ ID NO: 477 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgcccaagtctccaacattt

SEQ ID NO: 478 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gttggagacttgggcaatctt

SEQ ID NO: 479 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tattgcccaagtctccaactt

SEQ ID NO: 480 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggagacttgggcaatgtgctt

SEQ ID NO: 481 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacattgcccaagtctcctt

SEQ ID NO: 482 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggcaatgtgactgctgacctt

SEQ ID NO: 483 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtcagcagtcacattgcctt

SEQ ID NO: 484 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
caatgtgactgctgacaactt

SEQ ID NO: 485 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttgtcagcagtcacattgtt

SEQ ID NO: 486 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgacaaagatggtgtggctt

SEQ ID NO: 487 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccacaccatctttgtcagtt

SEQ ID NO: 488 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgacaaagatggtgtggcctt

SEQ ID NO: 489 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgccacaccatctttgtcatt

SEQ ID NO: 490 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctcaggagaccattgcatctt

SEQ ID NO: 491 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatgcaatggtctcctgagtt

SEQ ID NO: 492 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaggagaccattgcatcctt

SEQ ID NO: 493 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgatgcaatggtctcctgatt

SEQ ID NO: 494 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agaccattgcatcattggctt

SEQ ID NO: 495 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccaatgatgcaatggtcttt

SEQ ID NO: 496 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaccattgcatcattggcctt

SEQ ID NO: 497 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgccaatgatgcaatggtctt

SEQ ID NO: 498 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
attgcatcattggccgcactt

SEQ ID NO: 499 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgcggccaatgatgcaattt

SEQ ID NO: 500 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cattggccgcacactggtctt

SEQ ID NO: 501 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taccagtgtgcggccaatgtt

SEQ ID NO: 502 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cgcacactggtggtccatctt

SEQ ID NO: 503 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatggaccaccagtgtgcgtt

SEQ ID NO: 504 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cacactggtggtccatgactt

SEQ ID NO: 505 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcatggaccaccagtgtgtt

SEQ ID NO: 506 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
acactggtggtccatgaactt

SEQ ID NO: 507 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttcatggaccaccagtgttt

SEQ ID NO: 508 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggtggtccatgaaaaagctt

SEQ ID NO: 509 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctttttcatggaccaccatt

SEQ ID NO: 510 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggtccatgaaaaagcagctt

SEQ ID NO: 511 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tctgctttttcatggaccatt

SEQ ID NO: 512 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaagcagatgacttgggcctt

SEQ ID NO: 513 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcccaagtcatctgcttttt

SEQ ID NO: 514 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcagatgacttgggcaaactt

SEQ ID NO: 515 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttgcccaagtcatctgctt

SEQ ID NO: 516 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgacttgggcaaaggtgctt

SEQ ID NO: 517 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacctttgcccaagtcattt

SEQ ID NO: 518 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgacttgggcaaaggtggctt

SEQ ID NO: 519 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccacctttgcccaagtcatt

SEQ ID NO: 520 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gacttgggcaaaggtggactt

SEQ ID NO: 521 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttccacctttgcccaagtctt

SEQ ID NO: 522 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtacaaagacaggaaacgctt

SEQ ID NO: 523 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgtttcctgtctttgtactt

SEQ ID NO: 524 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
acaaagacaggaaacgctctt

SEQ ID NO: 525 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagcgtttcctgtctttgttt

SEQ ID NO: 526 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
caaagacaggaaacgctgctt

SEQ ID NO: 527 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcagcgtttcctgtctttgtt

SEQ ID NO: 528 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aggaaacgctggaagtcgctt

SEQ ID NO: 529 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgacttccagcgtttccttt

SEQ ID NO: 530 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtcgtttggcttgtggtgctt

SEQ ID NO: 531 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaccacaagccaaacgactt

SEQ ID NO: 532 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgtttggcttgtggtgtctt

SEQ ID NO: 533 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacaccacaagccaaacgatt

SEQ ID NO: 534 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cgtttggcttgtggtgtactt

SEQ ID NO: 535 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttacaccacaagccaaacgtt

SEQ ID NO: 536 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtttggcttgtggtgtaactt

SEQ ID NO: 537 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttacaccacaagccaaactt

SEQ ID NO: 538 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttggcttgtggtgtaattctt

SEQ ID NO: 539 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taattacaccacaagccaatt

SEQ ID NO: 540 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggcttgtggtgtaattggctt

SEQ ID NO: 541 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccaattacaccacaagcctt

SEQ ID NO: 542 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gcttgtggtgtaattgggctt

SEQ ID NO: 543 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcccaattacaccacaagctt

SEQ ID NO: 544 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cttgtggtgtaattgggactt

SEQ ID NO: 545 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcccaattacaccacaagtt

SEQ ID NO: 546 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtggtgtaattgggatcctt

SEQ ID NO: 547 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgatcccaattacaccacatt

SEQ ID NO: 548 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtggtgtaattgggatcgctt

SEQ ID NO: 549 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcgatcccaattacaccactt

SEQ ID NO: 550 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggtgtaattgggatcgcctt

SEQ ID NO: 551 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgcgatcccaattacaccatt

SEQ ID NO: 552 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtaattgggatcgcccaactt

SEQ ID NO: 553 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttgggcgatcccaattactt

SEQ ID NO: 554 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taattgggatcgcccaatctt

SEQ ID NO: 555 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tattgggcgatcccaattatt

SEQ ID NO: 556 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aattgggatcgcccaatactt

SEQ ID NO: 557 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttattgggcgatcccaatttt

SEQ ID NO: 558 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
attgggatcgcccaataactt

SEQ ID NO: 559 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttattgggcgatcccaattt

SEQ ID NO: 560 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgggatcgcccaataaactt

SEQ ID NO: 561 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttattgggcgatcccaatt

SEQ ID NO: 562 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgggatcgcccaataaacctt

SEQ ID NO: 563 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtttattgggcgatcccatt

SEQ ID NO: 564 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gggatcgcccaataaacactt

SEQ ID NO: 565 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgtttattgggcgatccctt

SEQ ID NO: 566 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atcgcccaataaacattcctt

SEQ ID NO: 567 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgaatgtttattgggcgattt

SEQ ID NO: 568 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ccaataaacattcccttgctt

SEQ ID NO: 569 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaagggaatgtttattggtt

SEQ ID NO: 570 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
caataaacattcccttggctt

SEQ ID NO: 571 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccaagggaatgtttattgtt

SEQ ID NO: 572 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aataaacattcccttggactt

SEQ ID NO: 573 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttccaagggaatgtttatttt

SEQ ID NO: 574 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ataaacattcccttggatctt

SEQ ID NO: 575 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatccaagggaatgtttattt

SEQ ID NO: 576 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taaacattcccttggatgctt

SEQ ID NO: 577 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcatccaagggaatgtttatt

SEQ ID NO: 578 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaacattcccttggatgtctt

SEQ ID NO: 579 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacatccaagggaatgttttt

SEQ ID NO: 580 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aacattcccttggatgtactt

SEQ ID NO: 581 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttacatccaagggaatgtttt

SEQ ID NO: 582 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
attcccttggatgtagtcctt

SEQ ID NO: 583 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgactacatccaagggaattt

SEQ ID NO: 584 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cttggatgtagtctgaggctt

SEQ ID NO: 585 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcctcagactacatccaagtt

SEQ ID NO: 586 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgaggccccttaactcactt

SEQ ID NO: 587 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttgagttaaggggcctcagtt

SEQ ID NO: 588 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaggccccttaactcatcctt

SEQ ID NO: 589 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgatgagttaaggggcctctt

SEQ ID NO: 590 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aggccccttaactcatctctt

SEQ ID NO: 591 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagatgagttaaggggccttt

SEQ ID NO: 592 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ccccttaactcatctgttctt

SEQ ID NO: 593 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taacagatgagttaaggggtt

SEQ ID NO: 594 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cccttaactcatctgttactt

SEQ ID NO: 595 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaacagatgagttaagggtt

SEQ ID NO: 596 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ccttaactcatctgttatctt

SEQ ID NO: 597 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tataacagatgagttaaggtt

SEQ ID NO: 598 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cttaactcatctgttatcctt

SEQ ID NO: 599 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgataacagatgagttaagtt

SEQ ID NO: 600 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaactcatctgttatccctt

SEQ ID NO: 601 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggataacagatgagttaatt

SEQ ID NO: 602 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taactcatctgttatcctctt

SEQ ID NO: 603 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taggataacagatgagttatt

SEQ ID NO: 604 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aactcatctgttatcctgctt

SEQ ID NO: 605 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaggataacagatgagtttt

SEQ ID NO: 606 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gttatcctgctagctgtactt

SEQ ID NO: 607 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttacagctagcaggataactt

SEQ ID NO: 608 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgctagctgtagaaatgctt

SEQ ID NO: 609 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcatttctacagctagcagtt

SEQ ID NO: 610 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgctagctgtagaaatgtctt

SEQ ID NO: 611 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacatttctacagctagcatt

SEQ ID NO: 612 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gctgtagaaatgtatcctctt

SEQ ID NO: 613 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taggatacatttctacagctt

SEQ ID NO: 614 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgtagaaatgtatcctgctt

SEQ ID NO: 615 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaggatacatttctacagtt

SEQ ID NO: 616 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtagaaatgtatcctgactt

SEQ ID NO: 617 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcaggatacatttctacatt

SEQ ID NO: 618 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtagaaatgtatcctgatctt

SEQ ID NO: 619 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatcaggatacatttctactt

SEQ ID NO: 620 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaatgtatcctgataaacctt

SEQ ID NO: 621 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtttatcaggatacattttt

SEQ ID NO: 622 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtatcctgataaacattactt

SEQ ID NO: 623 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaatgtttatcaggatactt

SEQ ID NO: 624 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaaacactgtaatcttactt

SEQ ID NO: 625 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaagattacagtgtttaatt

SEQ ID NO: 626 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
actgtaatcttaaaagtgctt

SEQ ID NO: 627 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacttttaagattacagttt

SEQ ID NO: 628 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ctgtaatcttaaaagtgtctt

SEQ ID NO: 629 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacacttttaagattacagtt

SEQ ID NO: 630 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtaatcttaaaagtgtactt

SEQ ID NO: 631 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttacacttttaagattacatt

SEQ ID NO: 632 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtaatcttaaaagtgtaactt

SEQ ID NO: 633 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttacacttttaagattactt

SEQ ID NO: 634 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cttaaaagtgtaattgtgctt

SEQ ID NO: 635 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacaattacacttttaagtt

SEQ ID NO: 636 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacctgtagtgagaaactctt

SEQ ID NO: 637 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagtttctcactacaggtatt

SEQ ID NO: 638 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttatgatcacttggaagactt

SEQ ID NO: 639 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcttccaagtgatcataatt

SEQ ID NO: 640 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgatcacttggaagattctt

SEQ ID NO: 641 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taatcttccaagtgatcattt

SEQ ID NO: 642 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atcacttggaagatttgtctt

SEQ ID NO: 643 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tacaaatcttccaagtgattt

SEQ ID NO: 644 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tggaagatttgtatagttctt

SEQ ID NO: 645 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taactatacaaatcttccatt

SEQ ID NO: 646 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tataaaactcagttaaaactt

SEQ ID NO: 647 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttttaactgagttttatatt

SEQ ID NO: 648 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaactcagttaaaatgtcctt

SEQ ID NO: 649 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgacattttaactgagttttt

SEQ ID NO: 650 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gtctgtttcaatgacctgctt

SEQ ID NO: 651 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaggtcattgaaacagactt

SEQ ID NO: 652 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgacctgtattttgccactt

SEQ ID NO: 653 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttggcaaaatacaggtcattt

SEQ ID NO: 654 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
acctgtattttgccagacctt

SEQ ID NO: 655 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtctggcaaaatacaggttt

SEQ ID NO: 656 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cctgtattttgccagactctt

SEQ ID NO: 657 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagtctggcaaaatacaggtt

SEQ ID NO: 658 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taaatcacagatgggtatctt

SEQ ID NO: 659 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tatacccatctgtgatttatt

SEQ ID NO: 660 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atcacagatgggtattaactt

SEQ ID NO: 661 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttaatacccatctgtgattt

SEQ ID NO: 662 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcacagatgggtattaaactt

SEQ ID NO: 663 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttaatacccatctgtgatt

SEQ ID NO: 664 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
acagatgggtattaaactctt

SEQ ID NO: 665 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tagtttaatacccatctgttt

SEQ ID NO: 666 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cagatgggtattaaacttctt

SEQ ID NO: 667 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taagtttaatacccatctgtt

SEQ ID NO: 668 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agatgggtattaaacttgctt

SEQ ID NO: 669 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaagtttaatacccatcttt

SEQ ID NO: 670 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
atgggtattaaacttgtcctt

SEQ ID NO: 671 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgacaagtttaatacccattt

SEQ ID NO: 672 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taaacttgtcagaatttcctt

SEQ ID NO: 673 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgaaattctgacaagtttatt

SEQ ID NO: 674 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcattcaagcctgtgaatctt

SEQ ID NO: 675 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tattcacaggcttgaatgatt

SEQ ID NO: 676 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
cattcaagcctgtgaatactt

SEQ ID NO: 677 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttattcacaggcttgaatgtt

SEQ ID NO: 678 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aataaaaaccctgtatggctt

SEQ ID NO: 679 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tccatacagggtttttatttt

SEQ ID NO: 680 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ataaaaaccctgtatggcctt

SEQ ID NO: 681 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgccatacagggtttttattt

SEQ ID NO: 682 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaccctgtatggcacttactt

SEQ ID NO: 683 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttaagtgccatacagggtttt

SEQ ID NO: 684 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
accctgtatggcacttatctt

SEQ ID NO: 685 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tataagtgccatacagggttt

SEQ ID NO: 686 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaggctattaaaagaatcctt

SEQ ID NO: 687 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgattcttttaatagcctctt

SEQ ID NO: 688 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaagaatccaaattcaaactt

SEQ ID NO: 689 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttttgaatttggattcttttt

SEQ ID NO: 690 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gaatccaaattcaaactactt

SEQ ID NO: 691 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttagtttgaatttggattctt

SEQ ID NO: 692 - RNA, 21 nt, synthetic construct
  misc_feature 1..21: note = Description of Artificial Sequence: Synthetic oligonucleotide
  source 1..21: mol_type = other RNA
caatgtgactgctgacaaccc

SEQ ID NO: 693 - RNA, 21 nt, synthetic construct
  misc_feature 1..21: note = Description of Artificial Sequence: Synthetic oligonucleotide
  source 1..21: mol_type = other RNA
tttgtcagcagtcacattgtt

SEQ ID NO: 694 - RNA, 21 nt, synthetic construct
  misc_feature 1..21: note = Description of Artificial Sequence: Synthetic oligonucleotide
  source 1..21: mol_type = other RNA
caatgtgactgctgacaatcc

SEQ ID NO: 695 - RNA, 21 nt, synthetic construct
  misc_feature 1..21: note = Description of Artificial Sequence: Synthetic oligonucleotide
  source 1..21: mol_type = other RNA
caatgtgactgctgacaagcc

SEQ ID NO: 696 - RNA, 21 nt, synthetic construct
  misc_feature 1..21: note = Description of Artificial Sequence: Synthetic oligonucleotide
  source 1..21: mol_type = other RNA
caatgtgactgctgacaaacc

SEQ ID NO: 697 - RNA, 21 nt, synthetic construct
  misc_feature 1..21: note = Description of Artificial Sequence: Synthetic oligonucleotide
  source 1..21: mol_type = other RNA
caatgtgacagctgacaaacc

SEQ ID NO: 698 - RNA, 20 nt, synthetic construct
  misc_feature 1..20: note = Description of Artificial Sequence: Synthetic oligonucleotide
  source 1..20: mol_type = other RNA
caatgtgactgctgacaacc

SEQ ID NO: 699 - RNA, 22 nt, synthetic construct
  misc_feature 1..22: note = Description of Artificial Sequence: Synthetic oligonucleotide
  source 1..22: mol_type = other RNA
caatgtgactgctgacaatccc

SEQ ID NO: 700 - RNA, 21 nt, synthetic construct
  misc_feature 1..21: note = Description of Artificial Sequence: Synthetic oligonucleotide
  source 1..21: mol_type = other RNA
caatgtgactgctgacaacac

SEQ ID NO: 701 - RNA, 21 nt, synthetic construct
  misc_feature 1..21: note = Description of Artificial Sequence: Synthetic oligonucleotide
  source 1..21: mol_type = other RNA
tttgtcagcagtcacattgtc

SEQ ID NO: 702 - RNA, 21 nt, synthetic construct
  misc_feature 1..21: note = Description of Artificial Sequence: Synthetic oligonucleotide
  source 1..21: mol_type = other RNA
caatgtgactgctgacaaatc

SEQ ID NO: 703 - RNA, 21 nt, synthetic construct
  misc_feature 1..21: note = Description of Artificial Sequence: Synthetic oligonucleotide
  source 1..21: mol_type = other RNA
caatgtgactgctgacaattc

SEQ ID NO: 704 - RNA, 21 nt, synthetic construct
  misc_feature 1..21: note = Description of Artificial Sequence: Synthetic oligonucleotide
  source 1..21: mol_type = other RNA
tttgtcagcagtcacattgac

SEQ ID NO: 705 - RNA, 22 nt, synthetic construct
  misc_feature 1..22: note = Description of Artificial Sequence: Synthetic oligonucleotide
  source 1..22: mol_type = other RNA
caatgtgactgctgacaatccc

SEQ ID NO: 706 - RNA, 21 nt, synthetic construct
  misc_feature 1..21: note = Description of Artificial Sequence: Synthetic oligonucleotide
  source 1..21: mol_type = other RNA
cgacgaaggccgtgtgcgccc

SEQ ID NO: 707 - RNA, 21 nt, synthetic construct
  misc_feature 1..21: note = Description of Artificial Sequence: Synthetic oligonucleotide
  source 1..21: mol_type = other RNA
tcgcacacggccttcgtcgtt

SEQ ID NO: 708 - RNA, 21 nt, synthetic construct
  misc_feature 1..21: note = Description of Artificial Sequence: Synthetic oligonucleotide
  source 1..21: mol_type = other RNA
tgacttgggcaaaggtggccc

SEQ ID NO: 709 - RNA, 21 nt, synthetic construct
  misc_feature 1..21: note = Description of Artificial Sequence: Synthetic oligonucleotide
  source 1..21: mol_type = other RNA
tccacctttgcccaagtcatt

SEQ ID NO: 710 - RNA, 21 nt, synthetic construct
  misc_feature 1..21: note = Description of Artificial Sequence: Synthetic oligonucleotide
  source 1..21: mol_type = other RNA
aactcatctgttatcctgccc

SEQ ID NO: 711 - RNA, 21 nt, synthetic construct
  misc_feature 1..21: note = Description of Artificial Sequence: Synthetic oligonucleotide
  source 1..21: mol_type = other RNA
tcaggataacagatgagtttt

SEQ ID NO: 712 - RNA, 21 nt, synthetic construct
  misc_feature 1..21: note = Description of Artificial Sequence: Synthetic oligonucleotide
  source 1..21: mol_type = other RNA
ccccttaactcatctgttccc

SEQ ID NO: 713 - RNA, 21 nt, synthetic construct
  misc_feature 1..21: note = Description of Artificial Sequence: Synthetic oligonucleotide
  source 1..21: mol_type = other RNA
taacagatgagttaaggggtt

SEQ ID NO: 714 - RNA, 21 nt, synthetic construct
  misc_feature 1..21: note = Description of Artificial Sequence: Synthetic oligonucleotide
  source 1..21: mol_type = other RNA
cccttaactcatctgttaccc

SEQ ID NO: 715 - RNA, 21 nt, synthetic construct
  misc_feature 1..21: note = Description of Artificial Sequence: Synthetic oligonucleotide
  source 1..21: mol_type = other RNA
ttaacagatgagttaagggtt

SEQ ID NO: 716 - RNA, 21 nt, synthetic construct
  misc_feature 1..21: note = Description of Artificial Sequence: Synthetic oligonucleotide
  source 1..21: mol_type = other RNA
aactcatctgttatcttgccc

SEQ ID NO: 717 - RNA, 21 nt, synthetic construct
  misc_feature 1..21: note = Description of Artificial Sequence: Synthetic oligonucleotide
  source 1..21: mol_type = other RNA
gctgtggaaatgtatcttccc

SEQ ID NO: 718 - RNA, 21 nt, synthetic construct
  misc_feature 1..21: note = Description of Artificial Sequence: Synthetic oligonucleotide
  source 1..21: mol_type = other RNA
taggatacatttctacagctt

SEQ ID NO: 719 - RNA, 21 nt, synthetic construct
  misc_feature 1..21: note = Description of Artificial Sequence: Synthetic oligonucleotide
  source 1..21: mol_type = other RNA
tgacttgggcaaaggtgagcc

SEQ ID NO: 720 - RNA, 21 nt, synthetic construct
  misc_feature 1..21: note = Description of Artificial Sequence: Synthetic oligonucleotide
  source 1..21: mol_type = other RNA
ccccttaactcatctgttgcc

SEQ ID NO: 721 - RNA, 21 nt, synthetic construct
  misc_feature 1..21: note = Description of Artificial Sequence: Synthetic oligonucleotide
  source 1..21: mol_type = other RNA
cccttaactcatctgttagcc

SEQ ID NO: 722 - RNA, 21 nt, synthetic construct
  misc_feature 1..21: note = Description of Artificial Sequence: Synthetic oligonucleotide
  source 1..21: mol_type = other RNA
aactcatctgttatcttagcc

SEQ ID NO: 723 - RNA, 21 nt, synthetic construct
  misc_feature 1..21: note = Description of Artificial Sequence: Synthetic oligonucleotide
  source 1..21: mol_type = other RNA
gctgtggaaatgtatcttgcc

SEQ ID NO: 724 - RNA, 21 nt, synthetic construct
  misc_feature 1..21: note = Description of Artificial Sequence: Synthetic oligonucleotide
  source 1..21: mol_type = other RNA
tgacttgggcaaaggtaggcc

SEQ ID NO: 725 - RNA, 21 nt, synthetic construct
  misc_feature 1..21: note = Description of Artificial Sequence: Synthetic oligonucleotide
  source 1..21: mol_type = other RNA
ccccttaacacatctgttacc

SEQ ID NO: 726 - RNA, 21 nt, synthetic construct
  misc_feature 1..21: note = Description of Artificial Sequence: Synthetic oligonucleotide
  source 1..21: mol_type = other RNA
cccttaactgatctgttaacc

SEQ ID NO: 727 - RNA, 21 nt, synthetic construct
  misc_feature 1..21: note = Description of Artificial Sequence: Synthetic oligonucleotide
  source 1..21: mol_type = other RNA
aactcatctcttatcttgccc

SEQ ID NO: 728 - RNA, 21 nt, synthetic construct
  misc_feature 1..21: note = Description of Artificial Sequence: Synthetic oligonucleotide
  source 1..21: mol_type = other RNA
gctgtggaattgtatcttgcc

SEQ ID NO: 729 - RNA, 21 nt, synthetic construct
  misc_feature 1..21: note = Description of Artificial Sequence: Synthetic oligonucleotide
  source 1..21: mol_type = other RNA
tgacttggggaaaggtgagcc

SEQ ID NO: 730 - RNA, 21 nt, synthetic construct
  misc_feature 1..21: note = Description of Artificial Sequence: Synthetic oligonucleotide
  source 1..21: mol_type = other RNA
aactcatctgttatcttggcc

SEQ ID NO: 731 - RNA, 21 nt, synthetic construct
  misc_feature 1..21: note = Description of Artificial Sequence: Synthetic oligonucleotide
  source 1..21: mol_type = other RNA
ccccttaactcatttgttccc

SEQ ID NO: 732 - RNA, 21 nt, synthetic construct
  misc_feature 1..21: note = Description of Artificial Sequence: Synthetic oligonucleotide
  source 1..21: mol_type = other RNA
tgacttgggcaaaggtagccc

SEQ ID NO: 733 - RNA, 21 nt, synthetic construct
  misc_feature 1..21: note = Description of Artificial Sequence: Synthetic oligonucleotide
  source 1..21: mol_type = other RNA
tttgtcagcagtcacattgtc

SEQ ID NO: 734 - RNA, 19 nt, synthetic construct
  misc_feature 1..19: note = Description of Artificial Sequence: Synthetic oligonucleotide
  source 1..19: mol_type = other RNA
caatgtgactgctgacaaa

SEQ ID NO: 735 - RNA, 21 nt, synthetic construct
  misc_feature 1..21: note = Description of Artificial Sequence: Synthetic oligonucleotide
  source 1..21: mol_type = other RNA
gcaggtcctcactttaatgcc

SEQ ID NO: 736 - RNA, 21 nt, synthetic construct
  misc_feature 1..21: note = Description of Artificial Sequence: Synthetic oligonucleotide
  source 1..21: mol_type = other RNA
gattaaagtgaggacctgctt

SEQ ID NO: 737 - RNA, 21 nt, synthetic construct
  misc_feature 1..21: note = Description of Artificial Sequence: Synthetic oligonucleotide
  source 1..21: mol_type = other RNA
ggcaatgtgactgctgacccc

SEQ ID NO: 738 - RNA, 21 nt, synthetic construct
  misc_feature 1..21: note = Description of Artificial Sequence: Synthetic oligonucleotide
  source 1..21: mol_type = other RNA
tgtcagcagtcacattgcctt

SEQ ID NO: 739 - RNA, 21 nt, synthetic construct
  misc_feature 1..21: note = Description of Artificial Sequence: Synthetic oligonucleotide
  source 1..21: mol_type = other RNA
gcaggtcctcactttaattcc

SEQ ID NO: 740 - RNA, 21 nt, synthetic construct
  misc_feature 1..21: note = Description of Artificial Sequence: Synthetic oligonucleotide
  source 1..21: mol_type = other RNA
ggcaatgtgactgctgatgcc

SEQ ID NO: 741 - RNA, 21 nt, synthetic construct
  misc_feature 1..21: note = Description of Artificial Sequence: Synthetic oligonucleotide
  source 1..21: mol_type = other RNA
gcaggtcctcactttaatccc

SEQ ID NO: 742 - RNA, 21 nt, synthetic construct
  misc_feature 1..21: note = Description of Artificial Sequence: Synthetic oligonucleotide
  source 1..21: mol_type = other RNA
ggcaatgtgactgctgatacc

SEQ ID NO: 743 - RNA, 21 nt, synthetic construct
  misc_feature 1..21: note = Description of Artificial Sequence: Synthetic oligonucleotide
  source 1..21: mol_type = other RNA
gcaggtcctgactttaatccc

SEQ ID NO: 744 - RNA, 21 nt, synthetic construct
  misc_feature 1..21: note = Description of Artificial Sequence: Synthetic oligonucleotide
  source 1..21: mol_type = other RNA
ggcaatgtgtctgctgatacc

SEQ ID NO: 745 - RNA, 22 nt, synthetic construct
  misc_feature 1..22: note = Description of Artificial Sequence: Synthetic oligonucleotide
  source 1..22: mol_type = other RNA
gattaaagtgaggacctgcttt

SEQ ID NO: 746 - RNA, 22 nt, synthetic construct
  misc_feature 1..22: note = Description of Artificial Sequence: Synthetic oligonucleotide
  source 1..22: mol_type = other RNA
tgtcagcagtcacattgccttt

SEQ ID NO: 747 - RNA, 152 nt, synthetic construct
  misc_feature 1..152: note = Description of Artificial Sequence: Synthetic polynucleotide
  source 1..152: mol_type = other RNA
tttatgcctcatcctctgagtgctgaaggcttgctgtaggctgtatgctgcaatgtgactgctgacaaccctgtgacctggtttgtcagcagtcacattgttagtgtatgatgcctgttactagcattcacatggaacaaattgctgccgtg

SEQ ID NO: 748 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaaccctgtgacctggtttgtcagcagtcacattgttctgaggagcgccttgacagcagccatgggagggccgccccctacctcagtga

SEQ ID NO: 749 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaatcctgtgacctggtttgtcagcagtcacattgttctgaggagcgccttgacagcagccatgggagggccgccccctacctcagtga

SEQ ID NO: 750 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaagcctgtgacctggtttgtcagcagtcacattgttctgaggagcgccttgacagcagccatgggagggccgccccctacctcagtga

SEQ ID NO: 751 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaaacctgtgacctggtttgtcagcagtcacattgttctgaggagcgccttgacagcagccatgggagggccgccccctacctcagtga

SEQ ID NO: 752 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgacagctgacaaacctgtgacctggtttgtcagcagtcacattgttctgaggagcgccttgacagcagccatgggagggccgccccctacctcagtga

SEQ ID NO: 753 - RNA, 157 nt, synthetic construct
  misc_feature 1..157: note = Description of Artificial Sequence: Synthetic polynucleotide
  source 1..157: mol_type = other RNA
gtgctgggcggggggcggcgggccctcccgcagaacaccatgcgctcttcggaacaatgtgactgctgacaacctgtgacctggtttgtcagcagtcacattgttctgaggagcgccttgacagcagccatgggagggccgccccctacctcagtga

SEQ ID NO: 754 - RNA, 159 nt, synthetic construct
  misc_feature 1..159: note = Description of Artificial Sequence: Synthetic polynucleotide
  source 1..159: mol_type = other RNA
gtgctgggcggggggcggcgggccctcccgcagaacaccatgcgctcttcggaacaatgtgactgctgacaatccctgtgacctggtttgtcagcagtcacattgttctgaggagcgccttgacagcagccatgggagggccgccccctacctcagtga

SEQ ID NO: 755 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaaccctgtgatttggtttgtcagcagtcacattgttctgaggagcgccttgacagcagccatgggagggccgccccctacctcagtga

SEQ ID NO: 756 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaaccctataatttggtttgtcagcagtcacattgttctgaggagcgccttgacagcagccatgggagggccgccccctacctcagtga

SEQ ID NO: 757 - RNA, 159 nt, synthetic construct
  misc_feature 1..159: note = Description of Artificial Sequence: Synthetic polynucleotide
  source 1..159: mol_type = other RNA
gtgctgggcggggggcggcgggccctcccgcagaacaccatgcgctcttcggaacaatgtgactgctgacaacaccctgacccagttttgtcagcagtcacattgttctgaggagcgccttgacagcagccatgggagggccgccccctacctcagtga

SEQ ID NO: 758 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaaccctgtgacctggtttgtcagcagtcacattgttctgtggagcgccttgacagcagccatgggagggccgccccctacctcagtga

SEQ ID NO: 759 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaatcctgtgacctggtttgtcagcagtcacattgttctgtggagcgccttgacagcagccatgggagggccgccccctacctcagtga

SEQ ID NO: 760 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgacagctgacaaacctgtgacctggtttgtcagcagtcacattgttctgtggagcgccttgacagcagccatgggagggccgccccctacctcagtga

SEQ ID NO: 761 - RNA, 159 nt, synthetic construct
  misc_feature 1..159: note = Description of Artificial Sequence: Synthetic polynucleotide
  source 1..159: mol_type = other RNA
gtgctgggcggggggcggcgggccctcccgcagaacaccatgcgctcttcggaacaatgtgactgctgacaacaccctgacccagttttgtcagcagtcacattgttctgtggagcgccttgacagcagccatgggagggccgccccctacctcagtga

SEQ ID NO: 762 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacaatgtgactgctgacaagcctgtgacctggtttgtcagcagtcacattgttctgtggagcgccttgacagcagccatgggagggccgccccctacctcagtga

SEQ ID NO: 763 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacgacgaaggccgtgtgcgccctgtgacctggtcgcacacggccttcgtcgttctgaggagcgccttgacagcagccatgggagggccgccccctacctcagtga

SEQ ID NO: 764 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttgggcaaaggtggccctgtgacctggtccacctttgcccaagtcattctgaggagcgccttgacagcagccatgggagggccgccccctacctcagtga

SEQ ID NO: 765 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctgttatcctgccctgtgacctggtcaggataacagatgagttttctgaggagcgccttgacagcagccatgggagggccgccccctacctcagtga

SEQ ID NO: 766 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagacaatgtgactgctgacaacccgtctgcacctgtcactagtttgtcagcagtcacattgtttggccgtgtagtgctacccagcgctggctgcctcctcagcattgcaattcctctcccatctgggcaccagtcagctaccctggtgggaatctgggtagcc

SEQ ID NO: 767 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaccccttaactcatctgttccctgtgacctggtaacagatgagttaaggggttctgaggagcgccttgacagcagccatgggagggccgccccctacctcagtga

SEQ ID NO: 768 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacccttaactcatctgttaccctgtgacctggttaacagatgagttaagggttctgaggagcgccttgacagcagccatgggagggccgccccctacctcagtga

SEQ ID NO: 769 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctgttatcttgccctgtgacctggtcaggataacagatgagttttctgaggagcgccttgacagcagccatgggagggccgccccctacctcagtga

SEQ ID NO: 770 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagctgtggaaatgtatcttccctgtgacctggtaggatacatttctacagcttctgaggagcgccttgacagcagccatgggagggccgccccctacctcagtga

SEQ ID NO: 771 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttgggcaaaggtgagcctgtgacctggtccacctttgcccaagtcattctgaggagcgccttgacagcagccatgggagggccgccccctacctcagtga

SEQ ID NO: 772 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaccccttaactcatctgttgcctgtgacctggtaacagatgagttaaggggttctgaggagcgccttgacagcagccatgggagggccgccccctacctcagtga

SEQ ID NO: 773 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacccttaactcatctgttagcctgtgacctggttaacagatgagttaagggttctgaggagcgccttgacagcagccatgggagggccgccccctacctcagtga

SEQ ID NO: 774 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctgttatcttagcctgtgacctggtcaggataacagatgagttttctgaggagcgccttgacagcagccatgggagggccgccccctacctcagtga

SEQ ID NO: 775 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagctgtggaaatgtatcttgcctgtgacctggtaggatacatttctacagcttctgaggagcgccttgacagcagccatgggagggccgccccctacctcagtga

SEQ ID NO: 776 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttgggcaaaggtaggcctgtgacctggtccacctttgcccaagtcattctgaggagcgccttgacagcagccatgggagggccgccccctacctcagtga

SEQ ID NO: 777 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaccccttaactcatctgttccctgtgatttggtaacagatgagttaaggggttctgaggagcgccttgacagcagccatgggagggccgccccctacctcagtga

SEQ ID NO: 778 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacccttaactcatctgttaccctgtgatttggttaacagatgagttaagggttctgaggagcgccttgacagcagccatgggagggccgccccctacctcagtga

SEQ ID NO: 779 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctgttatcttgccctgtgatttggtcaggataacagatgagttttctgaggagcgccttgacagcagccatgggagggccgccccctacctcagtga

SEQ ID NO: 780 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagctgtggaaatgtatcttccctgtgatttggtaggatacatttctacagcttctgaggagcgccttgacagcagccatgggagggccgccccctacctcagtga

SEQ ID NO: 781 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttgggcaaaggtgagcctgtgatttggtccacctttgcccaagtcattctgaggagcgccttgacagcagccatgggagggccgccccctacctcagtga

SEQ ID NO: 782 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaccccttaacacatctgttacctgtgacctggtaacagatgagttaaggggttctgtggagcgccttgacagcagccatgggagggccgccccctacctcagtga

SEQ ID NO: 783 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacccttaactgatctgttaacctgtgacctggttaacagatgagttaagggttctgtggagcgccttgacagcagccatgggagggccgccccctacctcagtga

SEQ ID NO: 784 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctcttatcttgccctgtgacctggtcaggataacagatgagttttctgtggagcgccttgacagcagccatgggagggccgccccctacctcagtga

SEQ ID NO: 785 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagctgtggaattgtatcttgcctgtgacctggtaggatacatttctacagcttctgtggagcgccttgacagcagccatgggagggccgccccctacctcagtga

SEQ ID NO: 786 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttggggaaaggtgagcctgtgacctggtccacctttgcccaagtcattctgtggagcgccttgacagcagccatgggagggccgccccctacctcagtga

SEQ ID NO: 787 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaccccttaactcatctgttgcctgtgacctggtaacagatgagttaaggggttctgtggagcgccttgacagcagccatgggagggccgccccctacctcagtga

SEQ ID NO: 788 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaacccttaactcatctgttagcctgtgacctggttaacagatgagttaagggttctgtggagcgccttgacagcagccatgggagggccgccccctacctcagtga

SEQ ID NO: 789 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaaactcatctgttatcttggcctgtgacctggtcaggataacagatgagttttctgtggagcgccttgacagcagccatgggagggccgccccctacctcagtga

SEQ ID NO: 790 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagctgtggaaatgtatcttgcctgtgacctggtaggatacatttctacagcttctgtggagcgccttgacagcagccatgggagggccgccccctacctcagtga

SEQ ID NO: 791 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaatgacttgggcaaaggtaggcctgtgacctggtccacctttgcccaagtcattctgtggagcgccttgacagcagccatgggagggccgccccctacctcagtga

SEQ ID NO: 792 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagaccccttaactcatttgttcccgtctgcacctgtcactagtaacagatgagttaaggggtttggccgtgtagtgctacccagcgctggctgcctcctcagcattgcaattcctctcccatctgggcaccagtcagctaccctggtgggaatctgggtagcc

SEQ ID NO: 793 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagacccttaactcatctgttacccgtctgcacctgtcactagttaacagatgagttaagggtttggccgtgtagtgctacccagcgctggctgcctcctcagcattgcaattcctctcccatctgggcaccagtcagctaccctggtgggaatctgggtagcc

SEQ ID NO: 794 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagaaactcatctgttatcttgcccgtctgcacctgtcactagtcaggataacagatgagtttttggccgtgtagtgctacccagcgctggctgcctcctcagcattgcaattcctctcccatctgggcaccagtcagctaccctggtgggaatctgggtagcc

SEQ ID NO: 795 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagagctgtggaaatgtatcttcccgtctgcacctgtcactagtaggatacatttctacagctttggccgtgtagtgctacccagcgctggctgcctcctcagcattgcaattcctctcccatctgggcaccagtcagctaccctggtgggaatctgggtagcc

SEQ ID NO: 796 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagatgacttgggcaaaggtagcccgtctgcacctgtcactagtccacctttgcccaagtcatttggccgtgtagtgctacccagcgctggctgcctcctcagcattgcaattcctctcccatctgggcaccagtcagctaccctggtgggaatctgggtagcc

SEQ ID NO: 797 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcgggatttgtcagcagtcacattgtctgtgacctggcaatgtgactgctgacaaatcctgaggagcgccttgacagcagccatgggagggccgccccctacctcagtga

SEQ ID NO: 798 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagcaggtcctcactttaatgcctgtgacctgggattaaagtgaggacctgcttctgaggagcgccttgacagcagccatgggagggccgccccctacctcagtga

SEQ ID NO: 799 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaggcaatgtgactgctgacccctgtgacctggtgtcagcagtcacattgccttctgaggagcgccttgacagcagccatgggagggccgccccctacctcagtga

SEQ ID NO: 800 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagcaggtcctcactttaattcctgtgacctgggattaaagtgaggacctgcttctgaggagcgccttgacagcagccatgggagggccgccccctacctcagtga

SEQ ID NO: 801 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaggcaatgtgactgctgatgcctgtgacctggtgtcagcagtcacattgccttctgaggagcgccttgacagcagccatgggagggccgccccctacctcagtga

SEQ ID NO: 802 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagcaggtcctcactttaatccctgtgatttgggattaaagtgaggacctgcttctgaggagcgccttgacagcagccatgggagggccgccccctacctcagtga

SEQ ID NO: 803 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaggcaatgtgactgctgatacctgtgatttggtgtcagcagtcacattgccttctgaggagcgccttgacagcagccatgggagggccgccccctacctcagtga

SEQ ID NO: 804 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagcaggtcctgactttaatccctgtgacctgggattaaagtgaggacctgcttctgtggagcgccttgacagcagccatgggagggccgccccctacctcagtga

SEQ ID NO: 805 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaggcaatgtgtctgctgatacctgtgacctggtgtcagcagtcacattgccttctgtggagcgccttgacagcagccatgggagggccgccccctacctcagtga

SEQ ID NO: 806 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaagcaggtcctcactttaatccctgtgacctgggattaaagtgaggacctgcttctgtggagcgccttgacagcagccatgggagggccgccccctacctcagtga

SEQ ID NO: 807 - RNA, 158 nt, synthetic construct
  misc_feature 1..158: note = Description of Artificial Sequence: Synthetic polynucleotide
  source 1..158: mol_type = other RNA
gtgctgggcggggggcggcgggccctcccgcagaacaccatgcgctcttcggaaggcaatgtgactgctgatacctgtgacctggtgtcagcagtcacattgccttctgtggagcgccttgacagcagccatgggagggccgccccctacctcagtga

SEQ ID NO: 808 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagagcaggtcctcactttaatcccgtctgcacctgtcactaggattaaagtgaggacctgctttggccgtgtagtgctacccagcgctggctgcctcctcagcattgcaattcctctcccatctgggcaccagtcagctaccctggtgggaatctgggtagcc

SEQ ID NO: 809 - RNA, 260 nt, synthetic construct
  misc_feature 1..260: note = Description of Artificial Sequence: Synthetic polynucleotide
  source 1..260: mol_type = other RNA
gaagcaaagaaggggcagagggagcccgtgagctgagtgggccagggactgggagaaggagtgaggaggcagggccggcatgcctctgctgctggccagaggcaatgtgactgctgataccgtctgcacctgtcactagtgtcagcagtcacattgcctttggccgtgtagtgctacccagcgctggctgcctcctcagcattgcaattcctctcccatctgggcaccagtcagctaccctggtgggaatctgggtagcc

SEQ ID NO: 810 - RNA, 54 nt, synthetic construct
  misc_feature 1..54: note = Description of Artificial Sequence: Synthetic oligonucleotide
  source 1..54: mol_type = other RNA
tcctgaggagcgccttgacagcagccatgggagggccgccccctacctcagtga